(19) United States
(12) Patent Application Publication
(10) Pub. No.: US 2011/0014613 A1
(43) Pub. Date: Jan. 20, 2011

(54) Genotyping for Risk of Atherosclerosis

(76) Inventors: Elisabeth PFUETZNER-RIEHN, Mainz (DE); Stefan PRAUSE, Mainz (DE); Moritz EIDENS, Mainz (DE); Alexander WEISE, Gensingen (DE); Andreas PFUETZNER, Mainz (DE)

Correspondence Address: MORGAN, LEWIS & BOCKIUS, LLP (SF), ONE MARKET SPEAR STREET TOWER, SAN FRANCISCO, CA, 94105, US

(21) Appl. No.: 12/752,845

(22) Filed: Apr. 1, 2010

Related U.S. Application Data
(60) Provisional application No. 61/165,815, filed on Apr. 1, 2009.

(51) Int. Cl. C12Q 1/68

(52) U.S. Cl. 435/6

(57) ABSTRACT

The invention provides a kits, compositions and methods useful for determining atherosclerotic risk in a subject. In one aspect, the invention provides kit comprising a solid support comprising a capture probe set comprising a plurality of probes selected from (a) a probe selective for PTGS1, (b) a probe selective for PTGS2, (c) a probe selective for NOS3, (d) a probe selective for SERPINE1, (e) a probe selective for F5, (f) a probe selective for MTHFR, (g) a probe selective for ALOX5AP, (h) a probe selective for CETP, (i) a probe selective for APOE, (j) a probe selective for F2, (k) a probe selective for ACE, (l) a probe selective for LTA and (m) a probe selective for LPL.

### CROSS-REFERENCE TO RELATED APPLICATIONS

This application claims under 35 USC 119(a) the benefit of U.S. Application 61/165,815, filed Apr. 1, 2009, which is incorporated by reference in its entirety for all purposes.

### TECHNICAL FIELD

The field of the invention relates to pharmacogenetics and molecular detection.

### BACKGROUND

Genomic medicine is a new branch in medicine taking the genomic differences of patients into account to improve the safety and effectiveness of modern drugs and therapies. One tool in this context is the use of genotyping to assess the risk of developing certain diseases, for the purposes of prevention through intervention using required drug regimes, diet modification, and exercise.

Currently, several technologies are available for the genotyping of the human genes related to atherosclerosis. Most important in this context is sequencing and real-time PCR. Sequencing is mainly done according to Sanger's method, by applying fluorescence labelled ddNTPs, which incorporate into the DNA during amplification and thereby stop this reaction. After separation, each different nucleotide can be detected by a special reader using four different fluorophores. Real-time PCR is another method which can be used to detect mutations by means of melting curve analysis. The melting curve is related to fluorescence labelled, sequence specific probes, which melt differently depending on whether the target is a wildtype or a mutated DNA. The change of fluorescence signal can be detected by the real-time measuring instrument. Several protocols have been developed to detect different atherosclerosis DNA markers by these two methods.

Genotyping in an easy, fast and cost-effective manner remains a challenge, however. Only a small number of mutations can be detected quickly and cost-effectively by sequencing and real-time PCR. This small number of detectable alleles is not enough to accurately meet the clinical need to comprehensively assess the state of the patient, and thus effectively guide prevention and required drug regimes.

### SUMMARY OF INVENTION

The invention provides kits, compositions (such as macroarray chips) and methods for determining risk of diseases such as atherosclerosis. One advantage provided by various embodiments of the invention is the fast, easy and cost-effective determination of the presence of any medically relevant mutations for assessing the risk of developing atherosclerosis or related disorders. The mutation set disclosed herein may be considered complete within the boundaries of the current medical literature and state of the art. Use of any combination of additional probes with any combination of the probes disclosed herein for the determination of other mutations is also contemplated. Simple, compact, reasonably priced equipment can be used, facilitating the availability of testing in a larger number of laboratories. Since the macroarray chip can be integrated into a common 1.5 mL lab tube in some embodiments, no specialized equipment has to be purchased from the laboratory and lab personnel require no special training.

Furthermore, due to the utilization of a precipitation reaction for detection instead of fluorescence as done by the competitive technologies described above, reagents cost less. Moreover, the result of the genotyping can be read out by a cost-effective reader or a microscope, since due to the precipitation, the detection is principally based on colorimetry and no expensive fluorescence based detection system has to be used.

In one aspect, the invention provides a kit comprising a solid support comprising a capture probe set comprising a plurality of probes selected from (a) a probe selective for PTGS1, (b) a probe selective for PTGS2, (c) a probe selective for NOS3, (d) a probe selective for SERPINE1, (e) a probe selective for F5, (f) a probe selective for MTHFR, (g) a probe selective for ALOX5AP, (h) a probe selective for CETP, (i) a probe selective for APOE, (j) a probe selective for F2, (k) a probe selective for ACE, (l) a probe selective for LTA and (m) a probe selective for LPL.

In some embodiments, the capture probe set comprises (a) a probe selective for a G1006A allele of PTGS1, (b) a probe selective for a R8W allele of PTGS1, (c) a probe selective for a P17L allele of PTGS1, (d) a probe selective for a −765G/C allele of PTGS2, (e) a probe selective for a −786T/C allele of NOS3, (f) a probe selective for a E298D allele of NOS3, (g) a probe selective for a 4G/5G allele of SERPINE1, (h) a probe selective for a G1691A allele of F5, (i) a probe selective for a C677T allele of MTHFR, (j) a probe selective for a A1298C allele of MTHFR, (k) a probe selective for a HapAB allele of ALOX5AP, (l) a probe selective for a HapA allele of ALOX5AP, (m) a probe selective for a HapB allele of ALOX5AP, (n) a probe selective for a Taq1b allele of CETP, (o) a probe selective for a −629C/A allele of CETP, (p) a probe selective for a A1061G allele of CETP, (q) a probe selective for a A1163G allele of CETP, (r) a probe selective for a Cys112Arg allele of APOE, (s) a probe selective for a Arg158Cys allele of APOE, (t) a probe selective for a G20210A allele of F2, (u) a probe selective for a Ins/Del allele of ACE, (v) a probe selective for a 252A/G allele of LTA, (w) a probe selective for a 804C/A allele of LTA, (x) a probe selective for a D9N allele of LPL, (y) a probe selective for a S447X allele of LPL, and (z) a probe selective for a N291S allele of LPL.

In some embodiments, the capture probe set comprises (a) (i) a probe selective for a first G1006A allele of PTGS1 and (ii) a probe selective for a second G1006A allele of PTGS1; (b) (i) a probe selective for a first R8W allele of PTGS1 and (ii) a probe selective for a second R8W allele of PTGS1; (c) (i) a probe selective for a first P17L allele of PTGS1 and (ii) a probe selective for a second P17L allele of PTGS1; (d) (i) a probe selective for a first −765G/C allele of PTGS2 and (ii) a probe selective for a second −765G/C allele of PTGS2; (e) (i) a probe selective for a first −786T/C allele of NOS3 and (ii) a probe selective for a second −786T/C allele of NOS3; (f) (i) a probe selective for a first E298D allele of NOS3 and (ii) a probe selective for a second E298D allele of NOS3; (g) (i) a probe selective for a first 4G/5G allele of SERPINE1 and (ii) a probe selective for a second 4G/5G allele of SERPINE1; (h) (i) a probe selective for a first G1691A allele of F5 and (ii) a probe selective for a second G1691A allele of F5; (i) (i) a probe selective for a first C677T allele of MTHFR and (ii) a probe selective for a second C677T allele of MTHFR; (j) (i) a probe selective for a first Al298C allele of MTHFR and (ii) a probe selective for a second A1298C allele of MTHFR; (k) (i) a probe selective for a first HapAB allele of ALOX5AP and (ii) a probe selective for a second HapAB allele of ALOX5AP; (l) (i) a probe selective for a first HapA allele of ALOX5AP and (ii) a probe selective for a second HapA allele of ALOX5AP; (m) (i) a probe selective for a first HapB allele of ALOX5AP and (ii) a probe selective for a second HapB allele of ALOX5AP; (n) (i) a probe selective for a first Taq1b allele of CETP and (ii) a probe selective for a second Taq1b allele of CETP; (o) (i) a probe selective for a first −629C/A allele of CETP and (ii) a probe selective for a second −629C/A allele of CETP; (p) (i) a probe selective for a first A1061G allele of CETP and (ii) a probe selective for a second A1061G allele of CETP; (q) (i) a probe selective for a first A1163G allele of CETP and (ii) a probe selective for a second A1163G allele of CETP; (r) (i) a probe selective for a first Cys112Arg allele of APOE and (ii) a probe selective for a second Cys112Arg allele of APOE; (s) (i) a probe selective for a first Arg158Cys allele of APOE and (ii) a probe selective for a second Arg158Cys allele of APOE; (t) (i) a probe selective for a first G20210A allele of F2 and (ii) a probe selective for a second G20210A allele of F2; (u) (i) a probe selective for a first Ins/Del allele of ACE and (ii) a probe selective for a second Ins/Del allele of ACE; (v) (i) a probe selective for a first 252A/G allele of LTA and (ii) a probe selective for a second 252A/G allele of LTA; (w) (i) a probe selective for a first 804C/A allele of LTA and (ii) a probe selective for a second 804C/A allele of LTA; (x) (i) a probe selective for a first D9N allele of LPL and (ii) a probe selective for a second D9N allele of LPL; (y) (i) a probe selective for a first S447X allele of LPL and (ii) a probe selective for a second S447X allele of LPL; and (z) (i) a probe selective for a first N291S allele of LPL and (ii) a probe selective for a second N291S allele of LPL.

In some embodiments, each of the probes is an isolated nucleic acid comprising a sequence selected from SEQ ID NOS: 1-196 or its complement, wherein each of the isolated nucleic acids is characterized by a length of about 18 to about 50 nucleic acids.

In some embodiments, each of the probes is an isolated nucleic acid consisting of a sequence selected from SEQ ID NOS: 1-196 or its complement.

In some embodiments, the capture probe set consists of a plurality of nucleic acids having sequences according to SEQ ID NOS: 1, 6, 9, 11, 12, 13, 15, 16, 18, 20, 22, 27, 28, 29, 30, 31, 36, 37, 39, 43, 44, 45, 46, 47, 50, 51, 54, 55, 56, 57, 58, 59, 60, 61, 62, 64, 65, 67, 68, 69, 70, 71, 72, 73, 74, 75, 76, 77, 78, 79, 82, 83, 84, 85, 86, 87, 88, 89, 90, 91, 92, 96, 99, 102, 103, 104, 105, 106, 107, 108, 109, 110, 111, 112, 113, 118, 119, 120, 121, 122, 127, 128, 129, 134, 135, 136, 138, 139, 140, 143, 144, 150, 151, 152, 153, 155, 156, 157, 158, 159, 160, 161, 166, 167, 168, 175, 176, 177, 178, 182, 183, 185, 186, 191, 191, 192, 192, 194, 196 and a combination selected from SEQ ID NOS: 2 and 3; 2 and 5; and 3 and 5.

In some embodiments, the kit further comprises a primer set comprising a plurality of primers selected from (a) a primer suitable for amplifying PTGS1, (b) a primer suitable for amplifying PTGS2, (c) a primer suitable for amplifying NOS3, (d) a primer suitable for amplifying SERPINE1, (e) a primer suitable for amplifying F5, (f) a primer suitable for amplifying MTHFR, (g) a primer suitable for amplifying ALOX5AP, (h) a primer suitable for amplifying CETP, (i) a primer suitable for amplifying APOE, (j) a primer suitable for amplifying F2, (k) a primer suitable for amplifying ACE, (l) a primer suitable for amplifying LTA and (m) a primer suitable for amplifying LPL.

In some embodiments, the primer set comprises a plurality of primer pairs selected from (a) a primer pair suitable for amplifying PTGS1, (b) a primer pair suitable for amplifying PTGS2, (c) a primer pair suitable for amplifying NOS3, (d) a primer pair suitable for amplifying SERPINE1, (e) a primer pair suitable for amplifying F5, (f) a primer pair suitable for amplifying MTHFR, (g) a primer pair suitable for amplifying ALOX5AP, (h) a primer pair suitable for amplifying CETP, (i) a primer pair suitable for amplifying APOE, (j) a primer pair suitable for amplifying F2, (k) a primer pair suitable for amplifying ACE, (1) a primer pair suitable for amplifying LTA and (m) a primer pair suitable for amplifying LPL.

In some embodiments, each of the primers is an isolated nucleic acid comprising a sequence selected from SEQ ID NOS: 197-248 or its complement, wherein each of the isolated nucleic acids is characterized by a length of about 17 to about 50 nucleic acids.

In some embodiments, each of the primers is an isolated nucleic acid consisting of a sequence selected from SEQ ID NOS: 197-248 or its complement.

In some embodiments, at least one of the plurality of primers comprises a detectable label.

In some embodiments, the detectable label is biotin.

In some embodiments, the kit further comprises a conjugated enzyme.

In some embodiments, the kit further comprises a precipitating agent.

In one aspect, the invention provides a method of detecting a plurality of alleles in a nucleic acid, the method comprising: (a) generating a plurality of amplicons in a sample comprising the nucleic acid, wherein the generating step comprises contacting the sample with a primer set of a kit disclosed herein and wherein each of the plurality of amplicons comprises a detectable label; (b) contacting the plurality of amplicons with the solid support of a kit disclosed herein; and (c) detecting the presence or absence of the detectable label, thereby detecting the plurality of alleles in the nucleic acid.

In some embodiments, the detecting step comprises contacting the sample with a conjugated enzyme.

In some embodiments, the detecting step comprises contacting the sample with a precipitating agent.

In some embodiments, the sample is derived from a subject experiencing or at risk of experiencing atherosclerosis.

In one aspect, the invention provides a method of assessing risk of atherosclerosis in a subject comprising: determining whether a nucleic acid in a sample from the subject is characterized by a plurality of gene variants selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

In some embodiments, the plurality of gene variants comprises a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

In some embodiments, the plurality of gene variants consists of a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

In some embodiments, the variant of PTGS1 is selected from G1006A, R8W and P17L; the variant of PTGS2 is −765G/C; the variant of NOS3 is selected from −786T/C and E298D; the variant of SERPINE1 is 4G/5G; the variant of F5 is G1691A; the variant of MTHFR is selected from C677T and Al298C; the variant of ALOX5AP is selected from HapAB, HapA and HapB; the variant of CETP is selected from Taq1b, −629C/A, A1061G and A1163G; the variant of APOE is selected from C112R and R158C; the variant of F2 is selected from G20210A; the variant of ACE is ins/del; the variant of LTA is selected from 252A/G and 804C/A or the variant of LPL is selected from D9N, S447X and N291 S.

In some embodiments, the determining step comprises: generating a plurality of amplicons in a sample comprising the nucleic acid, wherein the generating step comprises contacting the sample with a primer set comprising a plurality of primers suitable for amplifying the plurality of gene variants and wherein each of the plurality of amplicons comprises a detectable label; contacting the plurality of amplicons with a solid support comprising a plurality of capture probes selective for a plurality of variants selected from a variant of PTGS1, a variant of PTGS2, a variant of NOS3, a variant of SERPINE1 a variant of F5, a variant of MTHFR, a variant of ALOX5AP, a variant of CETP, a variant of APOE, a variant of F2, a variant of ACE, a variant of LTA and a variant of LPL; and detecting the presence or absence of the detectable label.

### BRIEF DESCRIPTION OF THE DRAWINGS

FIGS. 1A-1E show a number of capture probes for detecting the presence or absence of various alleles of various genes in a nucleic acid. The underlined base refers to a base at an “interrogation” position as described herein.

FIGS. 2A-2B show a number of primers useful in the various kits, compositions and methods described herein.

FIGS. 3A-3L show a number of sequences corresponding to various genes or portions thereof that are associated with risk of disease, such as atherosclerosis. An allele variation at a sequence position is indicated by “allelePos” and the nature of the allele is indicated by “alleles” in the header line, which begins with “>”. Thus, for each sequence record, each allele is contemplated and considered disclosed separately.

FIGS. 4 and 5A-5B show a pattern of probes on an example biochip.

FIG. 6 shows a typical result of selected probes.

### DESCRIPTION OF EMBODIMENTS

### Overview

The present invention provides kits, compositions and methods for detecting the presence or absence of various alleles of various genes in a target nucleic acid. The alleles are characterized by single nucleotide polymorphisms (SNPs), insertions, deletions or any combination thereof, all relative to a parent (e.g. wildtype, major allele or other allele) sequence. The investigated variations are connected to risks or states of disease, in particular atherosclerosis.

Analysis of a number of gene variants can aid in the assessment of disease (e.g., atherosclerosis) risk or status. Gene variants that are useful in determining risk or status of atherosclerosis include variants of one or more of the following genes: PTGS1, PTGS2, NOS3, SERPINE1, F5, MTHFR, ALOX5AP, CETP, APOE, F2, ACE, LTA and LPL. Genotyping a subject's DNA to detect an allele or variant of a number of these genes can help to optimize and individualize drug therapies for the subject, to prevent undesired effects and lower the costs that emerge from prolonged hospitalization and the treatment of adverse reactions.

In various aspects of the invention, the most predictive DNA markers to assess the risk of developing atherosclerosis and related diseases are tested and evaluated by a comprehensive meta analysis. In addition, these markers were used to develop a new predictive tool for point-of-care in-vitro diagnostics, which can be used to determine risk for atherosclerosis in a cost-effective, fast and easily-handled manner.

Accordingly, the present invention provides kits, compositions and methods for detecting the presence or absence of a nucleic acid sequence in a sample. The term “sample” used herein refers to a specimen or culture and includes liquids, gases and solids including for example tissue. In exemplary embodiments, a sample is obtained from a subject, for example, a mammal, preferably a human. A sample could be a fluid obtained from a subject including, for example, whole blood or a blood derivative (e.g. serum, plasma, or blood cells), ovarian cyst fluid, ascites, lymphatic, cerebrospinal or interstitial fluid, saliva, mucous, sputum, sweat, urine, or any other secretion, excretion, or other bodily fluids. As will be appreciated by those in the art, virtually any experimental manipulation or sample preparation steps may have been done on the sample. For example, wash steps may be applied to a sample. In an exemplary embodiment, the sample comprises blood, such as whole blood. In various embodiments, the sample comprises extracted nucleic acid. For example, the sample may be a buffer containing extracted nucleic acid. In various embodiments, a target sequence is measured directly in a subject without the need to obtain a separate sample from the patient.

If required, the target sequence is prepared using known techniques. For example, the sample may be treated to lyse the cells, using known lysis buffers, electroporation, etc., with purification and/or amplification as needed, as will be appreciated by those in the art. Suitable amplification techniques can be done, with PCR finding particular use in the invention as described herein.

### Kits

The invention provides kits useful for detecting the presence or absence of a nucleic acid (or nucleic acid sequence) in a sample. The term “nucleic acid”, “oligonucleotide” or “polynucleotide” herein means at least two nucleotides covalently linked together. A nucleic acid of the present invention will generally contain phosphodiester bonds, although in some cases (for example to stabilize the capture probes) the nucleic acids may have alternate backbones as known in the art.

Nucleic acids detected using the kits described herein may be referred to interchangeably as “target,” “target nucleic acid” or “target sequence.” A target sequence may be a portion or the entire length of a gene, a regulatory sequence, genomic DNA, cDNA, RNA including mRNA and rRNA, the complements of any of these and others. In exemplary embodiments, a target sequence is a portion of genomic DNA, especially a portion containing a sequence of an allele of a gene disclosed herein.

The target sequence may in some embodiments be a secondary target such as a product of an amplification reaction, such as PCR, (e.g. an “amplicon”) etc., as applied to, for example, a portion or the entire length of a gene, a regulatory sequence, genomic DNA, cDNA, RNA including mRNA and rRNA, the complements of any of these and the like. In some embodiments, the complement of a target sequence may be usefully detected and can provide the same information as detecting the target sequence. In some cases it is possible to detect an allele in a sense (i.e. plus) strand, antisense (i.e. minus) strand, or both, depending on the assay.

Target sequences may be of any length, with the understanding that longer sequences are more specific. As is outlined more fully below, capture probes are made to hybridize to target sequences to determine the presence or absence of the target sequence in a sample.

The target sequence may also be comprised of different target domains; for example, a first target domain of the sample target sequence may hybridize to a first capture probe and a second target domain may hybridize to a label probe (e.g. a “sandwich assay” format). The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the target sequence, the first target domain may be located either 5′ to the second domain, or 3′ to the second domain.

As is more fully outlined below, the target sequence comprises a position for which sequence information is desired, generally referred to herein as the “detection position.” In some embodiments, the detection position comprises a single nucleotide. In some embodiments, a detection position comprises a plurality of nucleotides, either contiguous with each other or separated by one or more nucleotides. In exemplary embodiments, the detection position in a target sequence corresponds to a gene variant or polymorphism that results in expression of a variant protein. As used herein, the base of a capture probe that basepairs with the detection position base in a hybrid is termed the “interrogation position.” In other words, for example, if a target sequence is an allele characterized by “A” or “G” at a polymorphic position, then the corresponding interrogation position in two capture probes would comprise “T” or “C” respectively.

Of particular use in the present invention are macroarray or biochip assays. By “macroarray”, “biochip” or “chip” herein is meant a composition generally comprising a solid support or substrate to which a capture probe is attached. Thus, in exemplary embodiments, the kits of the invention comprise a solid support. The term “solid support” or “substrate” refers to any material that can be modified to contain discrete individual sites appropriate for the attachment or association of a capture probe, described below. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers.

A number of different biochip array platforms as known in the art may be used. For example, the compositions and methods of the present invention can be implemented with array platforms such as GeneChip (Affymetrix), CodeLink Bioarray (Amersham), Expression Array System (Applied Biosystems), SurePrint microarrays (Agilent), Sentrix LD BeadChip or Sentrix Array Matrix (Illumina) and Verigene (Nanosphere).

Solid supports of particular use in the kits, compositions and methods of the present invention include those provided by ClonDiag™. In exemplary embodiments, a ClonDiag™ chip platform is used for the colorimetric detection of target sequences. That is, in some embodiments, the solid support comprises a ClonDiag™ chip. In various embodiments, a ClonDiag™ ArrayTube (AT) is used. One unique feature of the ArrayTube is the combination of a micro probe array (the biochip) and micro reaction vial. In various embodiments, where a target sequence is a nucleic acid, detection of the target sequence is done by amplifying and biotinylating the target sequence contained in a sample and optionally digesting the amplification products. The amplification product (amplicon) is then allowed to hybridize with capture probes contained on the ClonDiag™ chip and described below. A solution of a streptavidin-enzyme conjugate, such as Poly horseradish peroxidase (HRP) conjugate solution, is contacted with the ClonDiag™ chip. After washing, a dye solution such as o-dianisidine substrate solution is contacted with the chip. Oxidation of the dye results in precipitation that can be detected colorimetrically. Further description of the ClonDiag™ platform is found in Monecke S, Slickers P, Hotzel H et al., Clin Microbiol Infect 2006, 12: 718-728; Monecke S, Berger-Bachi B, Coombs C et al., Clin Microbiol Infect 2007, 13: 236-249; Monecke S, Leube I and Ehricht R, Genome Lett 2003, 2: 106-118; German Patent DE10201463; US Publication US/2005/0064469 and ClonDiag, ArrayTube (AT) Experiment Guideline for DNA-Based Applications, version 1.2, 2007, all incorporated by reference in their entirety. Examples of using the ClonDiag™ platform for genotyping is described in Sachse K et al., BMC Microbiology 2008, 8: 63; Monecke S and Ehricht R, Clin Microbiol Infect 2005, 11: 825-833; and Monecke S et al., Clin Microbiol Infect 2008, 14(6): 534-545. One of skill in the art will appreciate that numerous other dyes that react with a peroxidase can be utilized to produce a colorimetric change, such as 3,3′,5,5′-tetramethylbenzidine (TMB). For information on specific assay protocols, see www.clondiag.com/technologies/publications.php. Such dyes may be referred to as a “precipitating agent” herein. If solid supports other than the ClonDiag™ platform are used, attachment and immobilization of the capture probes are done according to methods known in the art.

In various exemplary embodiments, detection and measurement of target species utilizes colorimetric methods and systems in order to provide an indication of binding of a target species. In colorimetric methods, the presence of a bound target species will result in a change in the absorbance or transmission of light by a sample at one or more wavelengths. Detection of the absorbance or transmission of light at such wavelengths thus provides an indication of the presence of the target species.

In some embodiments, a detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element. In some embodiments, a colorimetric change is detected by inspection by the naked eye.

Transmission detection and analysis may be performed with a ClonDiag AT reader instrument. Suitable reader instruments and detection devices include the ArrayTube Workstation ATS and the ATR 03. In addition to ArrayTube, the ClonDiag ArrayStrip (AS) can be used. The ArrayStrip provides a 96-well format for high volume testing. Each ArrayStrip consists of a standard 8-well strip with a microarray integrated into the bottom of each well. Up to 12 ArrayStrips can be inserted into one microplate frame enabling the parallel multiparameter testing of up to 96 samples. The ArrayStrip can be processed using the ArrayStrip Processor ASP, which performs all liquid handling, incubation, and detection steps required in array based analysis.

The invention provides numerous kits for genotyping. Kits can be used for performing any of the methods disclosed herein for a number of medical (including diagnostic and therapeutic), industrial, forensic and research applications. Kits may comprise a portable carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of the various target species disclosed herein. For example, kits of the invention may also comprise, in the same or different containers, in any combination, one or more DNA polymerases, one or more primers, one or more probes, one or more binding ligands, one or more suitable buffers, one or more nucleotides (such as deoxynucleoside triphosphates (dNTPs) and preferably labeled dNTPs, such as biotin labeled dNTPs), one or more detectable labels and markers and one or more solid supports, any of which is described herein. The components may be contained within the same container, or may be in separate containers to be admixed prior to use. The kits of the present invention may also comprise one or more instructions or protocols for carrying out the methods of the present invention. The kits may comprise a detector for detecting a signal generated through use of the components of the invention in conjunction with a sample. The kits may also comprise a computer or a component of a computer, such as a computer-readable storage medium or device. Examples of storage media include, without limitation, optical disks such as CD, DVD and Blu-ray Discs (BD); magneto-optical disks; magnetic media such as magnetic tape and internal hard disks and removable disks; semi-conductor memory devices such as EPROM, EEPROM and flash memory; and RAM. The computer-readable storage medium may comprise software for data analysis or for encoding references to the various therapies, treatment regimens, risk classifications and instructions. The software may be interpreted by a computer to provide the practitioner with such information. Generally, any of the methods disclosed herein can comprise using any of the kits (comprising primers, probes, enzymes, labels, ligands, solid supports and other components, in any combination) disclosed herein.

### Probes

In exemplary embodiments, the kits of the invention comprise a solid support comprising a capture probe set. Capture probes sets comprise a plurality of “capture probes,” which are compounds used to detect the presence or absence of, or to quantify, relatively or absolutely, a target sequence. Generally, a capture probe allows the attachment of a target sequence to a solid support for the purposes of detection as further described herein. Attachment of the target species to the capture binding ligand can be direct or indirect and can be covalent or noncovalent. Capture probes that bind directly to a target may be said to be “selective” for, “specifically bind” or “selectively bind” their target. It should be noted that capture probes are designed to be perfectly or substantially complementary to either strand (e.g. either the sense or the antisense strand) of a double stranded polynucleotide, such as a gene. Thus, in some cases, a capture probe of the invention is perfectly or substantially complementary to the sense strand; that is, assuming the sense strand is referred to as “Watson”, the capture probe would be “Crick”. In some cases, a capture probe of the invention is perfectly or substantially complementary to the antisense strand.

Capture probes that “selectively bind” to or are “selective for” (i.e., are “complementary” or “substantially complementary” to) a target nucleic acid find use in the present invention. “Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules may be said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98% to 100%, and in some embodiments, at least a percentage is selected from 90%, 91%, 92%, 93%, 94%, 95%, 96%, 97%, 98% and 99%. Where one single stranded RNA or DNA molecule is shorter than another, the two single stranded RNA or DNA molecules may be said to be substantially complementary when the nucleotides of the longer strand, optimally aligned and compared and with appropriate nucleotide insertions or deletions, pair with at least about 80% of the nucleotides of the shorter strand, usually at least about 90% to 95%, and more preferably from about 98% to 100%, and in some embodiments, at least a percentage is selected from 90%, 91%, 92%, 93%, 94%, 95%, 96%, 97%, 98% and 99%. Alternatively, substantial complementarity exists (i.e., one sequence is selective for another) when an RNA or DNA strand will hybridize under selective hybridization conditions (for example, stringent conditions or high stringency conditions as known in the art) to its complement. Typically, selective hybridization will occur when there is at least about 65% complementarity over a stretch of at least 14 to 25 nucleotides, preferably at least about 75%, more preferably at least about 90% (or 91%, 92%, 93%, 94%, 95%, 96%, 97%, 98% or 99%) complementarity. See, M. Kanehisa, Nucleic Acids Res., 2004, 12: 203. In some embodiments, the term “bind” refers to binding under high stringency conditions. In some embodiments, a capture probe that selectively binds to or is selective for a target is perfectly complementary to the target. In some embodiments, a capture probe that selectively binds to or is selective for a target is substantially complementary to the target.

The invention provides numerous capture probe sets that can attached to a solid support. Such capture probe sets are useful for determining risk or status of a disease. Each of the probes of the capture probe set should be complementary to at least a portion of a gene. In some embodiments, a capture probe set comprises a plurality of probes that are used to detect all medially relevant mutations (for example, all those relevant to atherosclerosis) in selected genes. In one embodiment, a capture probe set comprises a plurality of probes selected from (a) a probe selective for PTGS1, (b) a probe selective for PTGS2, (c) a probe selective for NOS3, (d) a probe selective for SERPINE1, (e) a probe selective for F5, (f) a probe selective for MTHFR, (g) a probe selective for ALOX5AP, (h) a probe selective for CETP, (i) a probe selective for APOE, (j) a probe selective for F2, (k) a probe selective for ACE, (l) a probe selective for LTA and (m) a probe selective for LPL. A capture probe set can comprise or consist of any combination of these probes.

In exemplary embodiments, each of the probes of the capture probe set is suitable for distinguishing at least two different alleles of a given gene, such as a gene disclosed herein. The probes or capture probe sets provided by the invention can be used to determine polymorphism at a gene locus. As understood in the art, an “allele” refers to a particular alternative form of a gene. For convenience, the term “allele” as used herein can also refer to a combination of alleles at multiple loci that are transmitted together on the same chromosome. That is, an allele can refer to a haplotype. An allele can be characterized, for example, by substitution, insertion or deletion of one or more bases relative to a different allele. A capture probe could thus, in various examples, span a polymorphic site of the gene, span one or more insertions or span nucleic acids flanking a deletion.

In one embodiment, a capture probe set comprises a probe that is selective for an allele of a gene. In one embodiment, a capture probe set comprises a pair of probes, one of which is selective for a first allele of a gene and one of which is selective for a second allele of the gene. In some embodiments, a capture probe set comprises a pair of probes, one of which is selective for a wildtype allele of the gene and one of which is selective for a mutant (or “variant”) allele of the gene. The term “wildtype” can in some embodiments refer to a major allele or an allele that is the most frequently occurring allele. The term “variant” can in some embodiments refer to a minor allele or an allele that is not the most frequently occurring allele. In exemplary embodiments, a capture probe set comprises a pair of probes, one of which is selective for a major allele of the gene and one of which is selective for a minor allele of the gene. In some embodiments, a capture probe set comprises more than two probes, each of which is selective for a different allele of the gene. In exemplary embodiments, a capture probe set comprises one or more probes selective for one or more alleles of one or more genes.

In various embodiments, one, two, three, four, five or six or more probes are used to probe a chosen gene or allele. In various embodiments, the number of probes used to probe a first allele is different from the number of probes used to probe a second allele. In various embodiments, each of the chosen alleles is probed by the same number of probes. Furthermore, additional alleles as known in the art may probed in any combination with any combination of the alleles disclosed herein, using any combination of primers and probes. In various embodiments, either the forward or reverse sequence of an allele may be probed, i.e., a given sequence corresponding to an allele or its complement may be probed.

In various embodiments, at least two probes are used to detect each different gene. In an exemplary embodiment, a capture probe set comprises a plurality of probe pairs selected from (a) a pair of probes comprising a probe selective for a first allele of PTGS1 and a probe selective for a second allele of PTGS1, (b) a pair of probes comprising a probe selective for a first allele of PTGS2 and a probe selective for a second allele of PTGS2, (c) a pair of probes comprising a probe selective for a first allele of NOS3 and a probe selective for a second allele of NOS3, (d) a pair of probes comprising a probe selective for a first allele of SERPINE1 and a probe selective for a second allele of SERPINE1, (e) a pair of probes comprising a probe selective for a first allele of F5 and a probe selective for a second allele of F5, (f) a pair of probes comprising a probe selective for a first allele of MTHFR and a probe selective for a second allele of MTHFR, (g) a pair of probes comprising a probe selective for a first allele of ALOX5AP and a probe selective for a second allele of ALOX5AP, (h) a pair of probes comprising a probe selective for a first allele of CETP and a probe selective for a second allele of CETP, (i) a pair of probes comprising a probe selective for a first allele of APOE and a probe selective for a second allele of APOE, (j) a pair of probes comprising a probe selective for a first allele of F2 and a probe selective for a second allele of F2, (k) a pair of probes comprising a probe selective for a first allele of ACE and a probe selective for a second allele of ACE, (l) a pair of probes comprising a probe selective for a first allele of LTA and a probe selective for a second allele of LTA, and (m) a pair of probes comprising a probe selective for a first allele of LPL and a probe selective for a second allele of LPL. Thus, any combination of genes selected from PTGS1, PTGS2, NOS3, SERPINE1, F5, MTHFR, ALOX5AP, CETP, APOE, F2, ACE, LTA and LPL may be probed, and for each gene of the combination, any combination of alleles may be probed using any number of probes. In some embodiments, the first allele of a gene is a wildtype allele. In some embodiments, the second allele of a gene is a variant or mutant allele. In some embodiments, where a first capture probe and a second capture probe are used to determine the presence of a first allele containing a substitution, insertion or deletion relative to a second allele, the first capture probe is selective for the first allele and the second capture probe is selective for the second allele. In some embodiments, the first capture probe has a low binding affinity for the second allele or a lower binding affinity relative to the second capture probe for the second allele; similarly, the second capture probe has a low binding affinity for the first allele or a lower binding affinity relative to the first capture probe for the first allele. In some embodiments, the first capture probe is perfectly complementary to the first allele and is not perfectly complementary to the second allele, and the second capture probe is perfectly to the second allele and is not perfectly complementary to the first allele.

Table 1 shows exemplary alleles that can be probed to provide information about a subject's atherosclerotic risk or status. In order to identify the probe for each mutation, a complex experimental evaluation was performed. This ensures a most robust assay. Thus, fewer probes have to be spotted on the macroarray chip compared to other technologies and production costs decrease enormously. Numerous alleles or variants disclosed in Table 1 have been found to be associated with atherosclerotic risk.

TABLE 1

| | Allele (by | Allele (by | |
|---|---|---|---|
| | reference to | reference to | |
| | nucleic acid | amino acid | dbSNP record |
| Gene | substitution) | substitution) | number |
| PTGS1 | G1006A | | |
| | | R8W | rs1236913 |
| | | P17L | rs3842787 |
| PTGS2 | −765G/C | | rs20417 |
| NOS3 | −786T/C | | rs2070744 |
| | | E298D | rs1799983 |
| SERPINE1 | 4G/5G | | rs1799889 |
| F5 | G1691A | | rs6025 |
| MTHFR | C677T | | rs1801133 |
| | A1298C | | rs1801131 |
| ALOX5AP | HapAB | | rs10507391 |
| | HapA | | rs17222814, |
| | | | rs10507391, |
| | | | rs4769874, |
| | | | rs9551963 |
| | HapB | | rs17216473, |
| | | | rs10507391, |
| | | | rs9315050, |
| | | | rs17222842 |
| CETP | Taq1b | | rs708272 |
| | −629C/A | | rs1800775 |
| | A1061G | | rs5882 |
| | A1163G | | rs2303790 |
| APOE | | C112R | rs429358 |
| | | R158C | rs7412 |
| F2 | G20210A | | rs1799963 |
| ACE | ins/del | | rs13447447 |
| LTA | 252A/G | | rs909253 |
| | 804C/A | | rs1041981 |
| LPL | | D9N | rs1801177 |
| | | S447X | rs328 |
| | | N291S | rs268 |

As can be seen in Table 1, an allele may be referred to in various ways. For example, an allele may be referred to by a substitution of a nucleotide for another in a parent polynucleotide strand (e.g., genomic DNA, mRNA, fragments thereof, amplication products thereof and other polynucleotides disclosed herein) or by the substitution of an amino acid for another in a parent polypeptide strand (e.g., a polypeptide resulting from translation of a polynucleotide). In some instances, a reference to an amino acid substitution corresponds to a nucleotide variation in the gene that causes that amino acid substitution in the polypeptide resulting from expression of the gene as understood in the art. Where reference is made to a substitution, both a parent molecule (e.g. gene) and a molecule containing the substitution relative to the parent are contemplated and either allele may be probed. Where reference is made to an insertion, both a parent molecule (e.g. gene) and a molecule containing the insertion relative to the parent is contemplated and either allele may be probed. Where reference is made to a deletion, both a parent molecule (e.g. gene) and a molecule containing the deletion relative to the parent is contemplated and either allele may be probed.

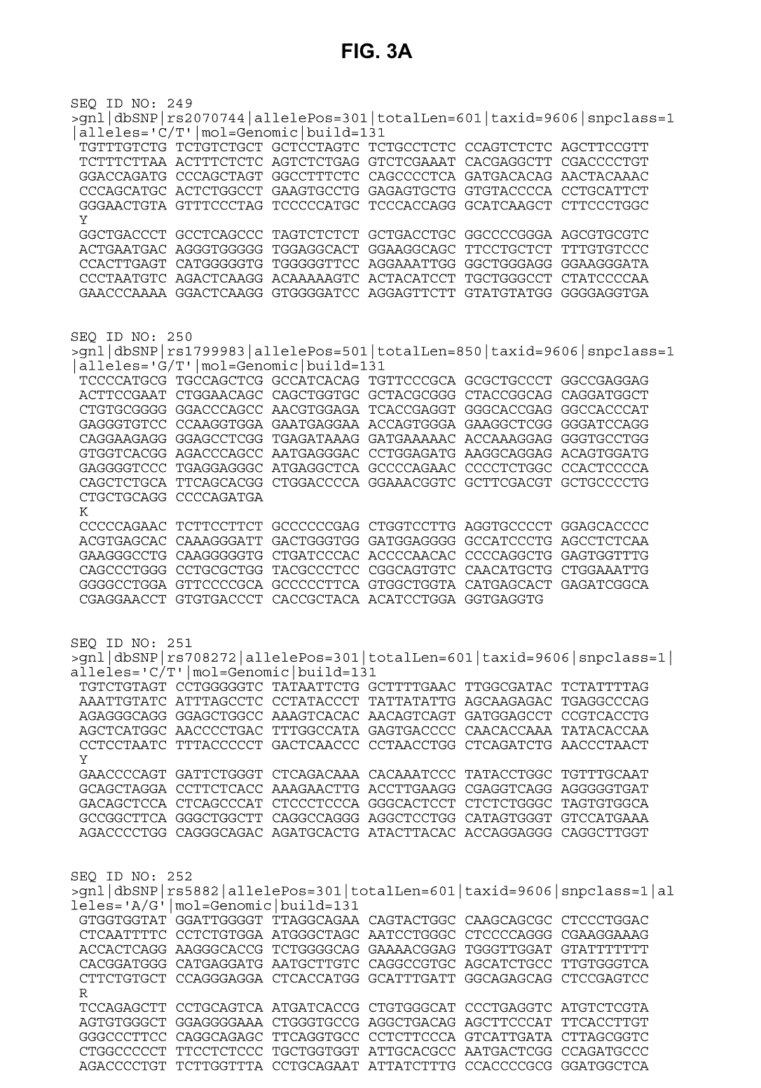

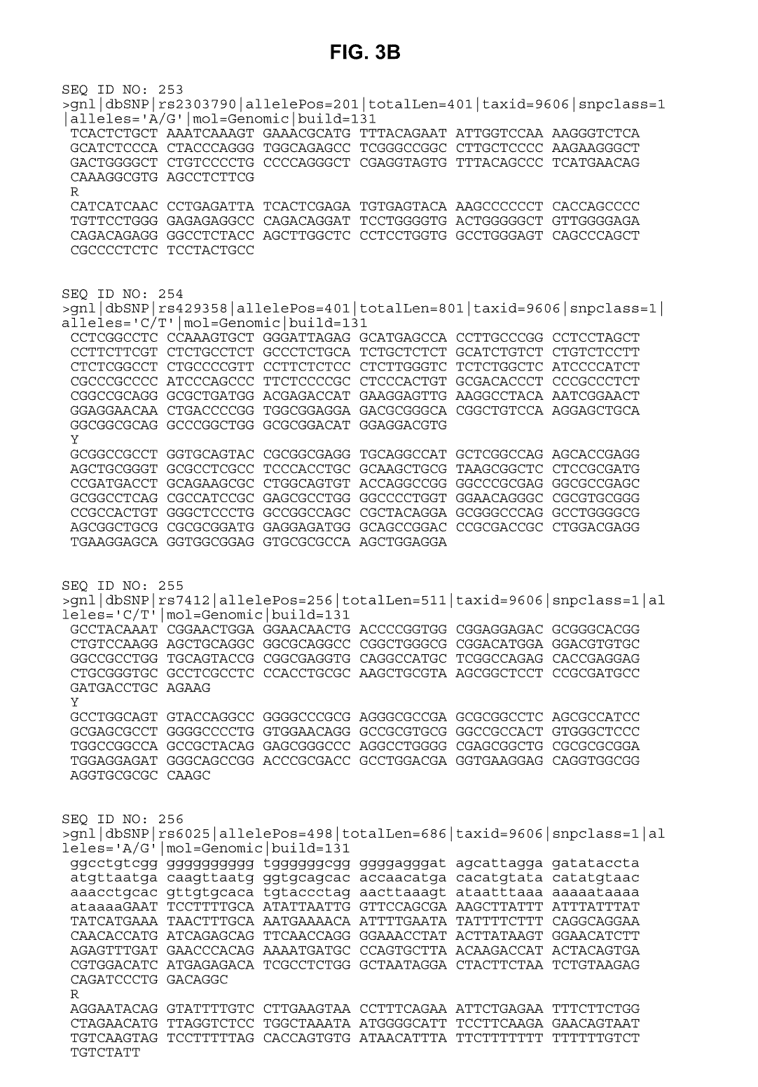

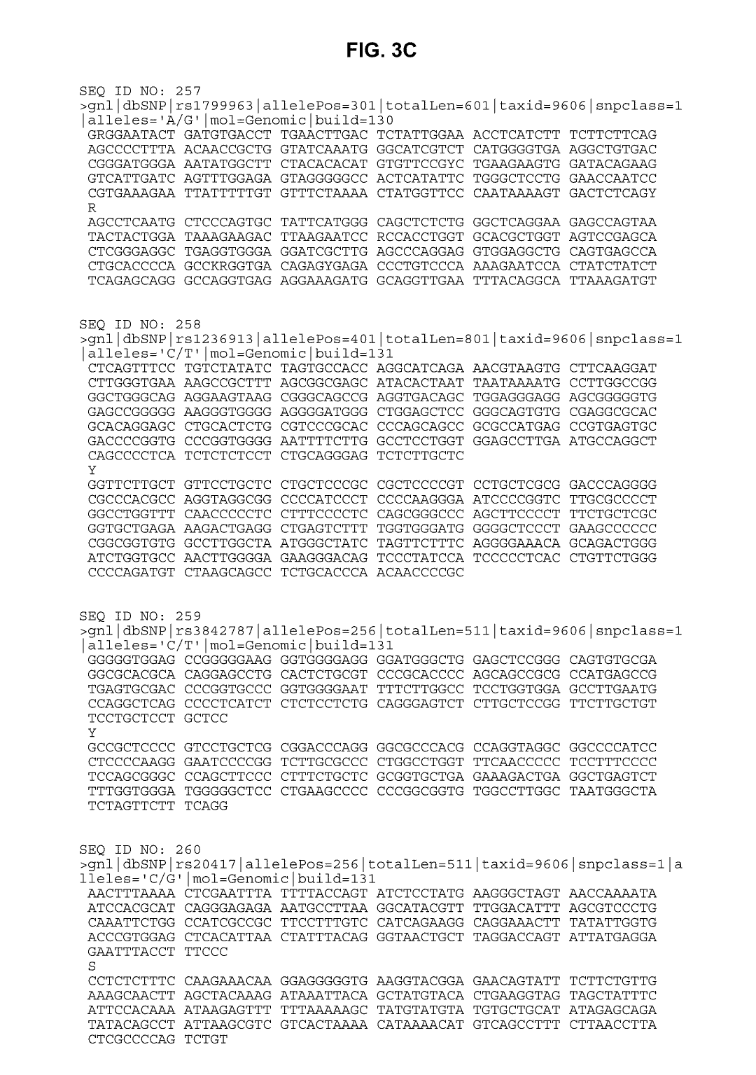

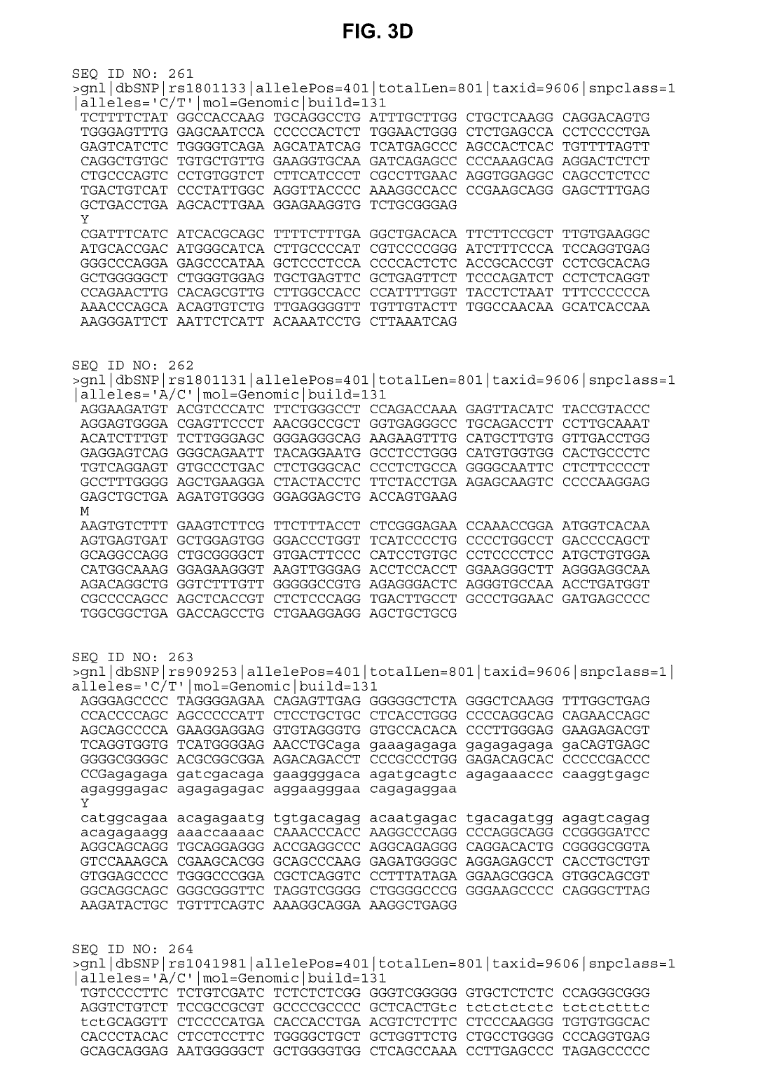

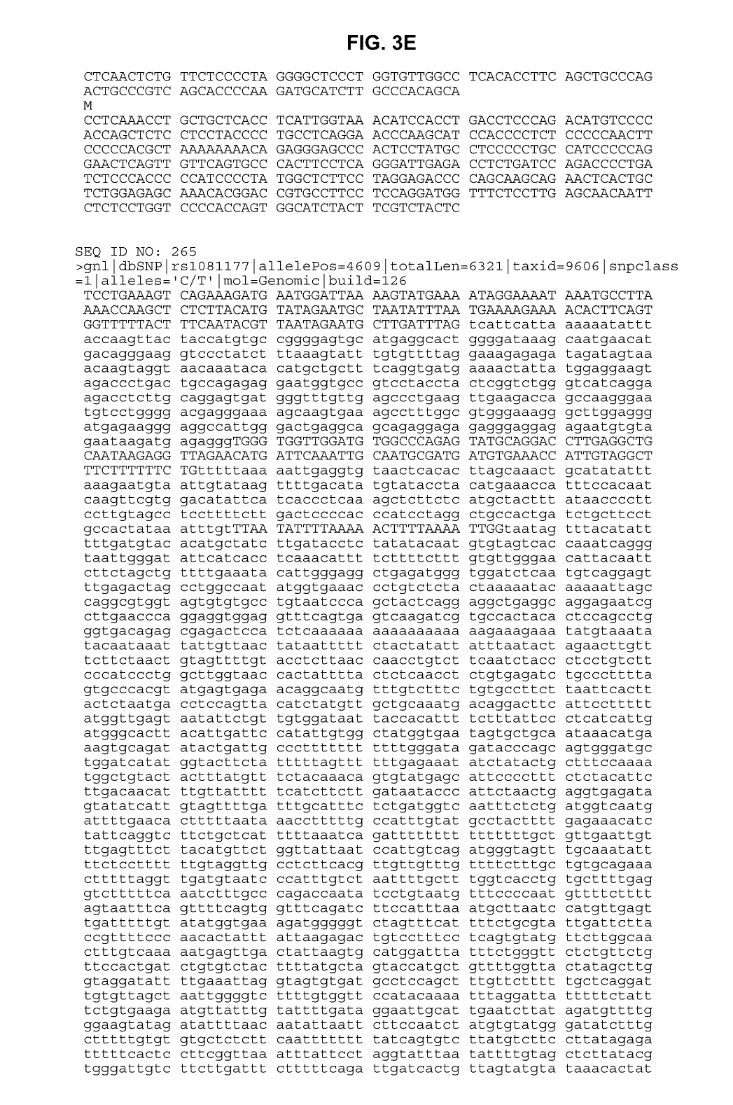

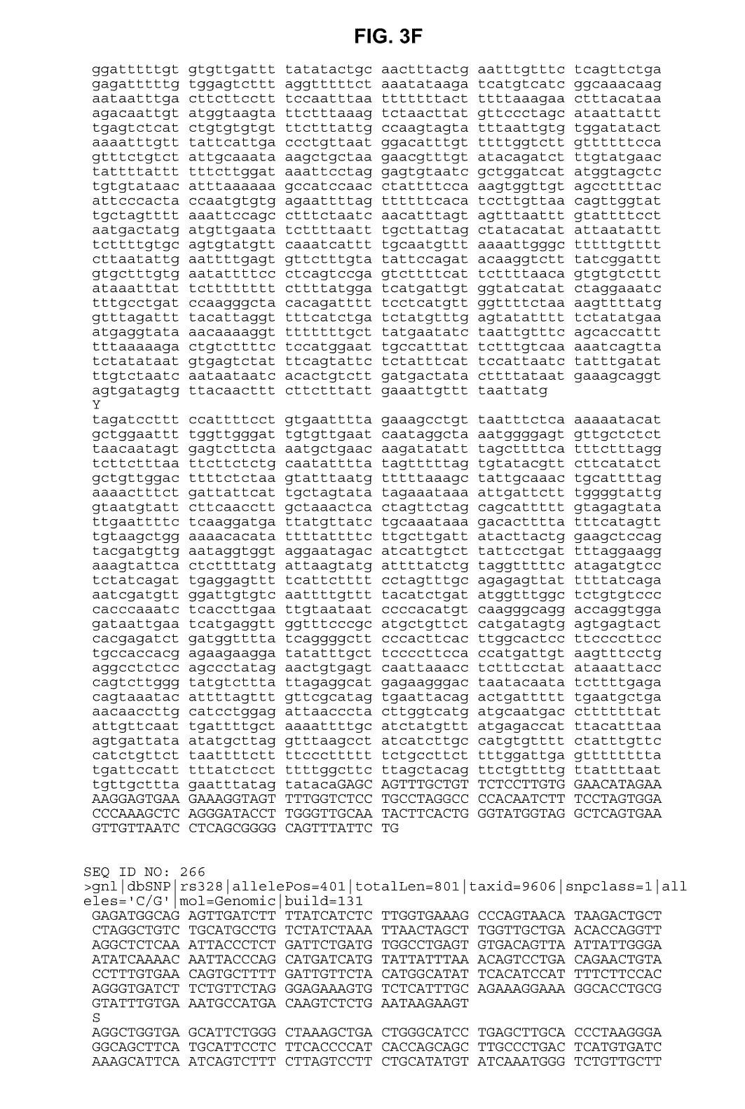

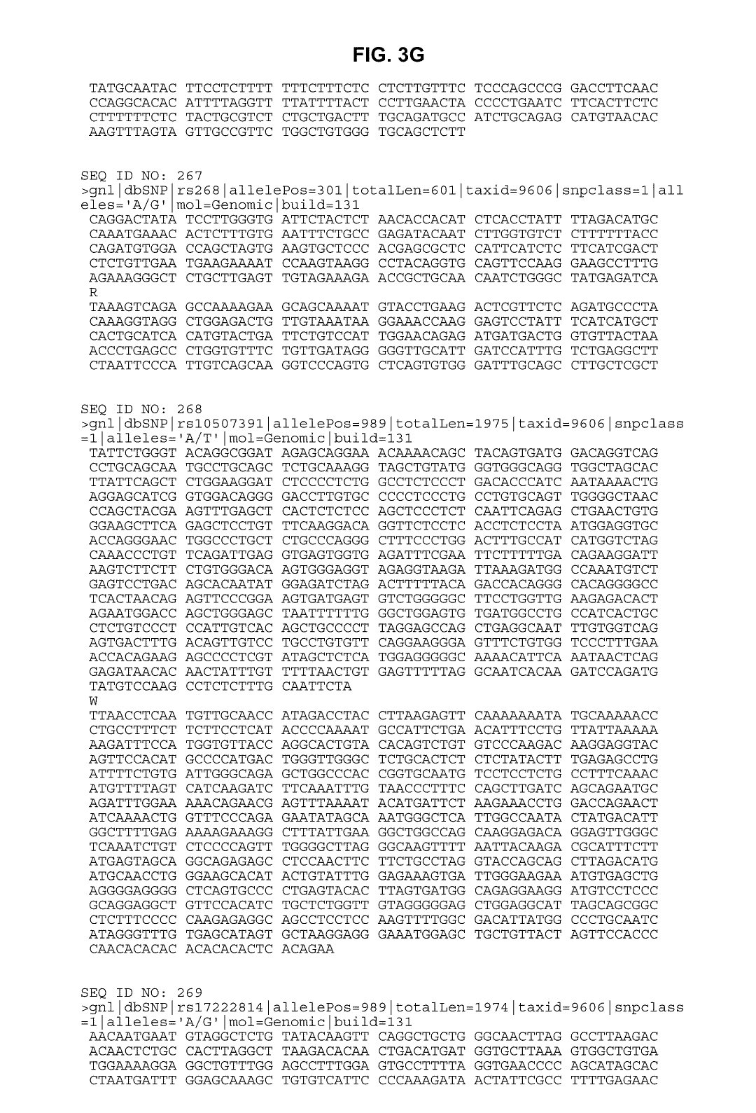

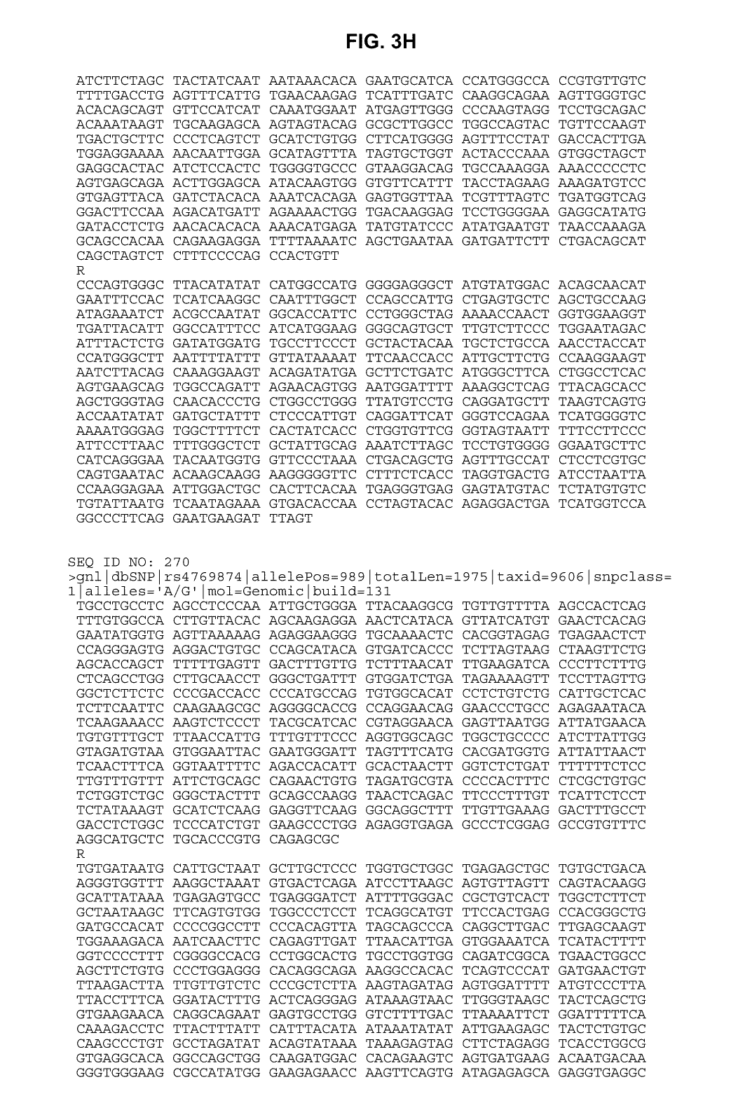

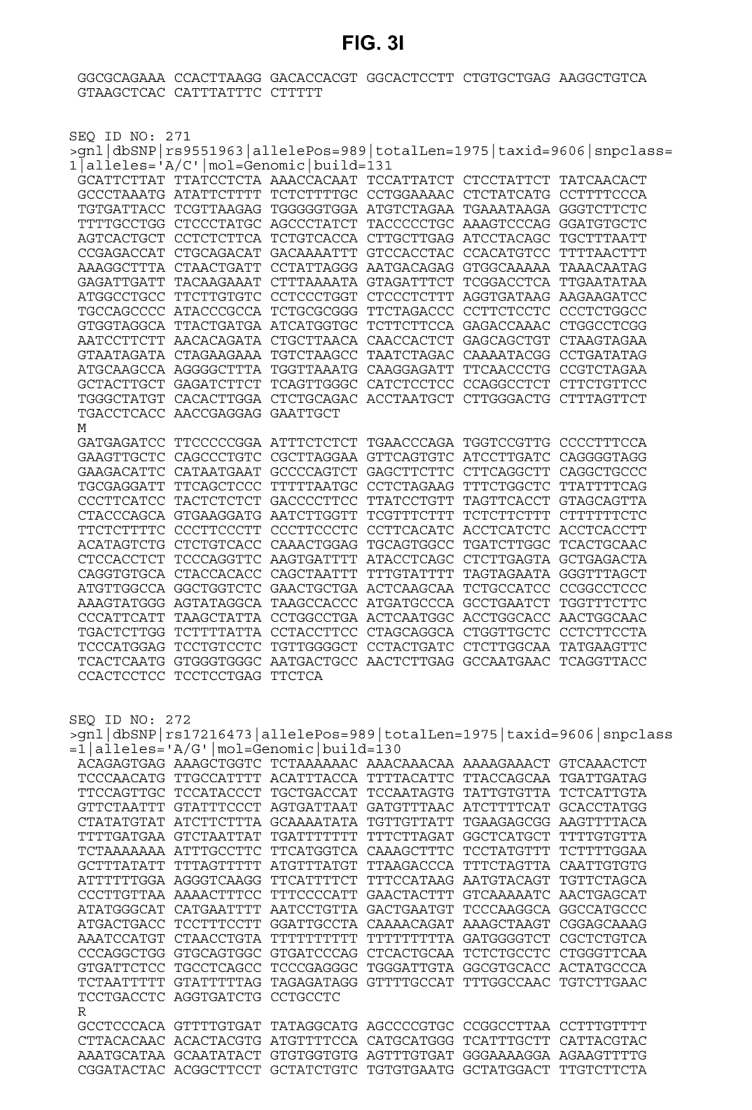

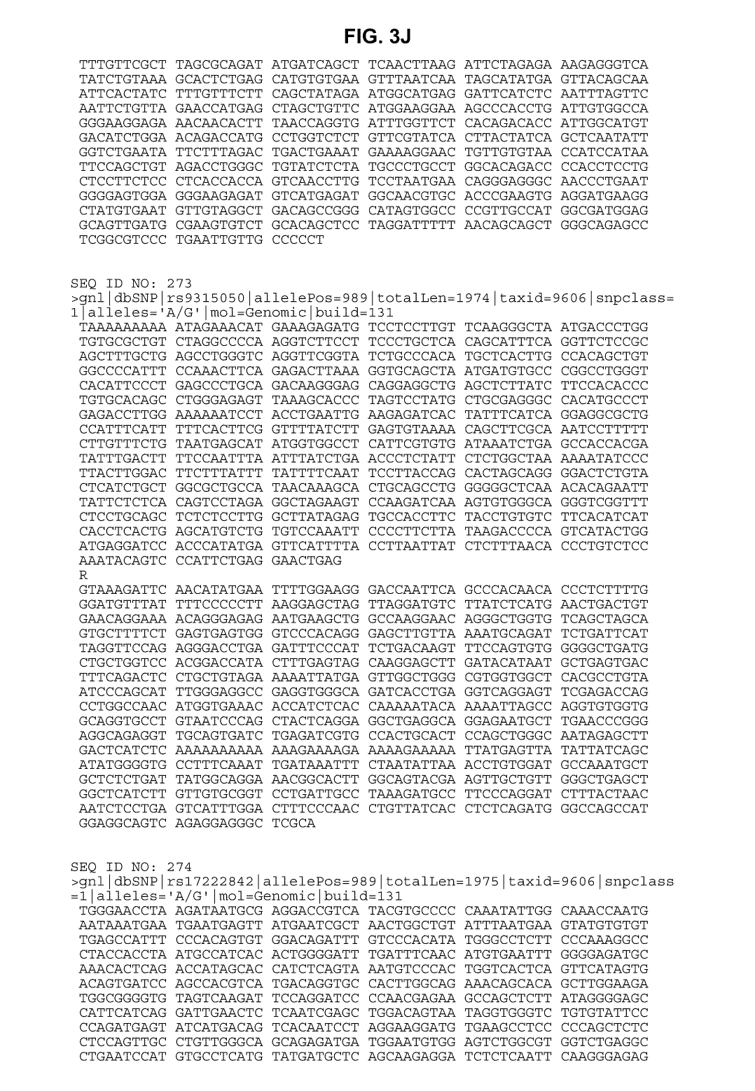

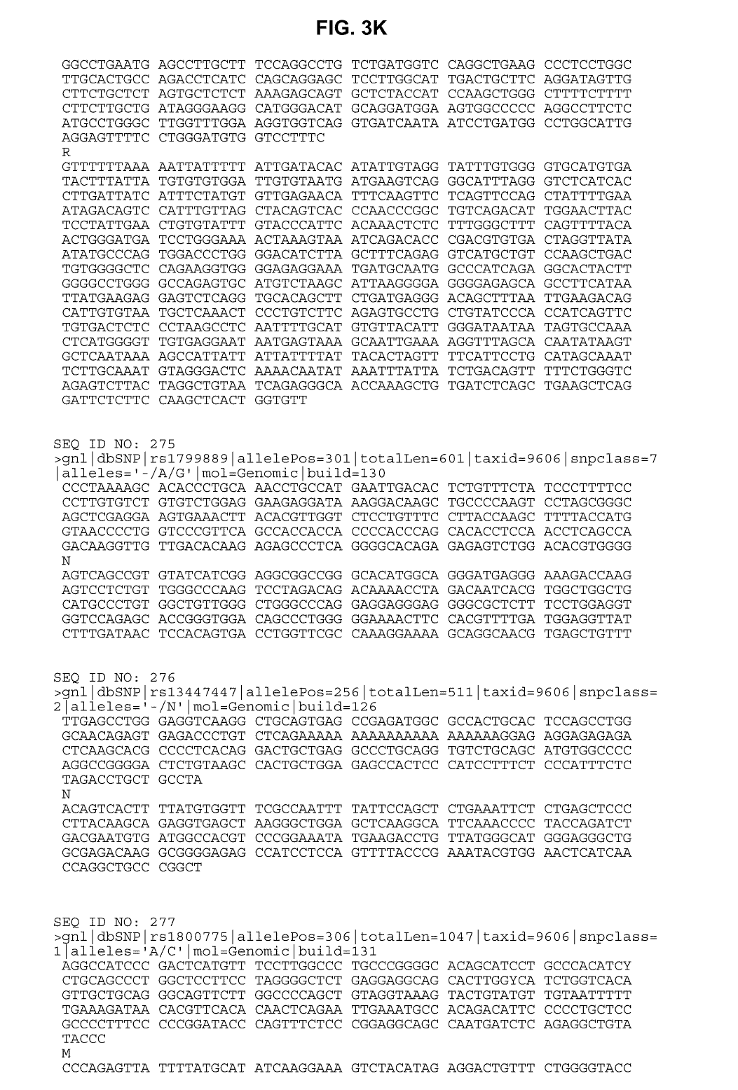

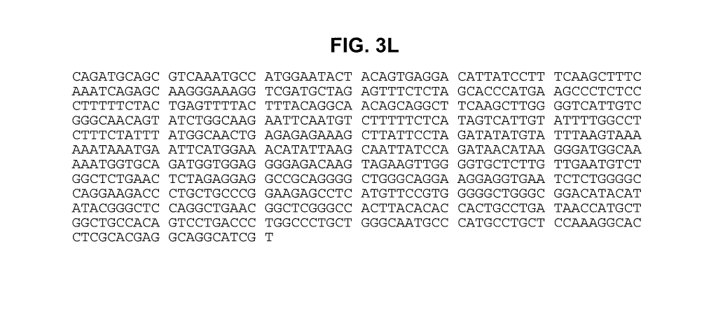

Thus, an allele may be referred to by a reference to a substitution, insertion or deletion of one or more nucleic acids or a substitution, insertion or deletion of one or more amino acids. The “Taq1b” allele refers to the presence of a Taq1 restriction site. An allele of a gene can also be referred to by a dbSNP rs record number, such as those shown in Table 1. Where multiple rs record numbers are given for an allele, a sequence in any rs record or a combination of sequences in a combination of rs records can be probed. Example sequences from dbSNP are shown in FIGS. 3A-3L. Table 1 refers to alleles that are understood in the art. Any of the alleles as referred to by any type of reference in Table 1 can be probed in any combination. In various embodiments, any combination of the alleles disclosed herein may be probed.

The gene names in Table 1 are official, but other names can also be used for the same gene. For example, “eNOS” as used herein refers to NOS3; “prothrombin” refers to F2; “AloxAP” refers to ALOX5AP and “PA1” refers to SERPINE1.

In some embodiments, one or more capture probes are used to identify the base at a detection position. In these embodiments, each different probe comprises a different base at an “interrogation position,” which will differentially hybridize to the detection position of the target sequence. By using different probes, each with a different base at the interrogation position, the identification of the base at the detection position is elucidated. In some embodiments, a capture probe does not comprise an interrogation position. Such embodiments might be useful for detecting deletion or insertion variants. For example, in some embodiments, a capture probe for a wildtype allele comprises an interrogation position, and a capture probe for a deletion mutant of the allele does not comprise the interrogation position. In some embodiments, an interrogation position in a capture probe for detecting a deletion variant corresponds to a nucleic acid deleted from a parent (e.g. wildtype) polynucleotide. In some embodiments, a capture probe for a wildtype allele does not comprise an interrogation position, and a capture probe for an insertion mutant of the allele comprises an interrogation position. In some embodiments, an interrogation position in a capture probe for detecting an insertion variant corresponds to a nucleic acid inserted into a parent (e.g. wildtype) polynucleotide.

In one embodiment, all nucleotides outside of the interrogation position in two or more probes are the same; that is, in some embodiments it is preferable to use probes that have equal all components other than the interrogation position (e.g. both the length of the probes as well as the non-interrogation bases) to allow good discrimination. In some embodiments, it may be desirable to alter other components, in order to maximize discrimination at the detection position. For example, all nucleotides outside of the interrogation position in two probes may be the same except for one or two nucleic acids added to the end of only one probe.

As a preliminary matter, the strand that gives the most favorable difference for Tₘ differences is preferably chosen: G/T is chosen over C/A and G/A over C/T mismatches, for example. In some embodiments, probes are used that have the interrogation base in the middle region of the probe, rather than towards one of the ends. However, as outlined herein, the shifting of the interrogation position within the probe can be used to maximize discrimination in some embodiments.

For example, in a preferred embodiment, the perfect match/mismatch discrimination of the probes may be enhanced by changing the binding affinities of bases at and near the mismatch position. For example, sequences that have G-C pairs adjacent to the detection position (or within 3 bases) can hinder good discrimination of match/mismatch. By choosing substitutions in these areas, better discrimination is achieved. For example, this may be done to either destabilize the base pairing in the detection position, or preferably to stabilize the base pairing in the detection position while destabilizing the base pairs in the positions adjacent to the detection position. Base substitutions reduce the number of hydrogen bonds to only two or less hydrogen bonds per base pair without disturbing the stacking structure of the double strand in the area. The amount of destabilization will depend on the chemical nature of the substitution, the number of substitutions and the position of the substitutions relative to the detection position. The local strand destabilization has to be balanced against the loss of specificity of the probe. These substitutions can be either naturally occurring or synthetic base analogs as known in the art.

In exemplary embodiments, the discrimination of the capture probes can be altered by altering the length of the probes. For example, as noted above, certain mismatches, such as G/A differences, can be difficult due to the stability of G:T mispairings. By decreasing the standard probe length from 15-25 basepairs to 10-15 basepairs, increased discrimination may be done.

In addition, matching the Tₘs of the different capture probes with their complements allows for good multiplexing; that is, the panel of different alleles to be evaluated need to tested under one set of conditions, and thus the capture probes are designed accordingly.

Thus, the invention provides capture probes comprising an interrogation position, and in some cases not comprising an interrogation position, that can be used to identify the nucleotide at a number of detection positions within various genes or fragments thereof In exemplary embodiments, the nucleotide at a detection position corresponds to a SNP of an allele. A capture probe comprising an interrogation position can be used to detect an insertion, deletion or substitution of a nucleotide relative to a parent (e.g., wildtype or variant) nucleic acid.

In some embodiments, a capture probe comprising an interrogation position is perfectly complementary to a fragment of a target sequence outside of the corresponding detection position. A capture probe can thus be constructed by identifying an interrogation position and extending a number of nucleotides in the 5′ direction and a number of nucleotides in the 3′ direction. In some embodiments, the extension in either or both directions will be perfectly complementary to a portion of a target sequence. For example, in reference to a sequence indicated in a dbSNP rs record, the portion of a target sequence can be the nucleotides outside of the polymorphic position (“allelepos”) indicated in the sequence. In this way, it could be said that the capture probe spans the polymorphic position. The capture probe can be of any length that permits differential hybridization compared to a second capture probe having the same length but a different nucleotide at the interrogation position. In some embodiments, a capture probe is perfectly complementary to a sequence indicated in a dbSNP rs record. In some embodiments, a capture probe is substantially complementary to a sequence indicated in a dbSNP rs record. In some embodiments, a capture probe is pefectly complementary to a sequence indicated in a dbSNP rs record outside of a polymorphic position. In some embodiments, a capture probe is substantially complementary to a sequence indicated in a dbSNP rs record outside of a polymorphic position.

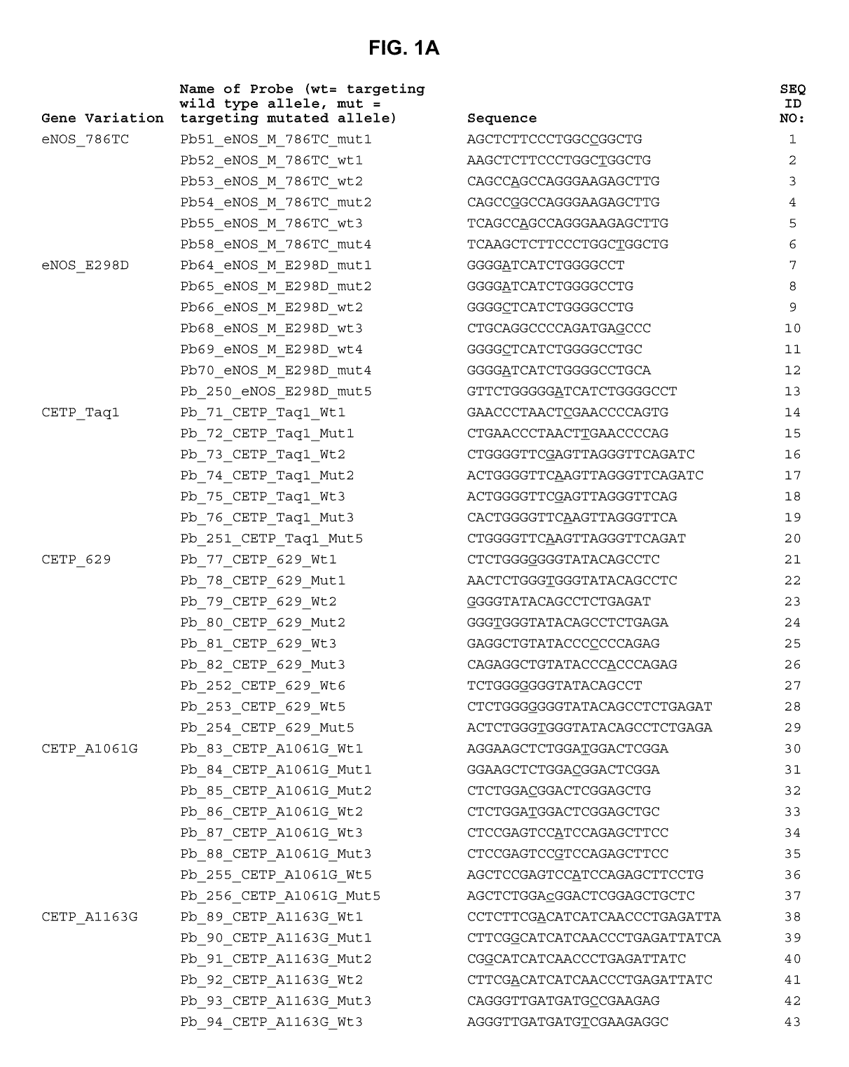

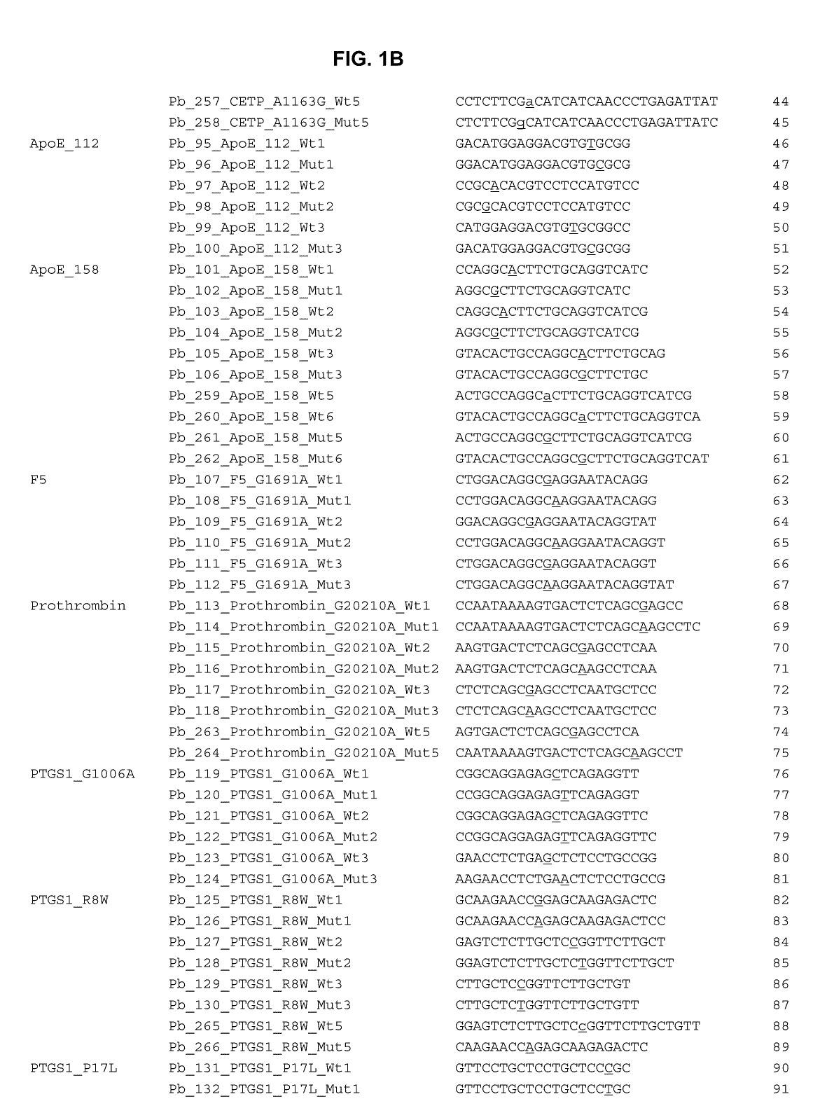

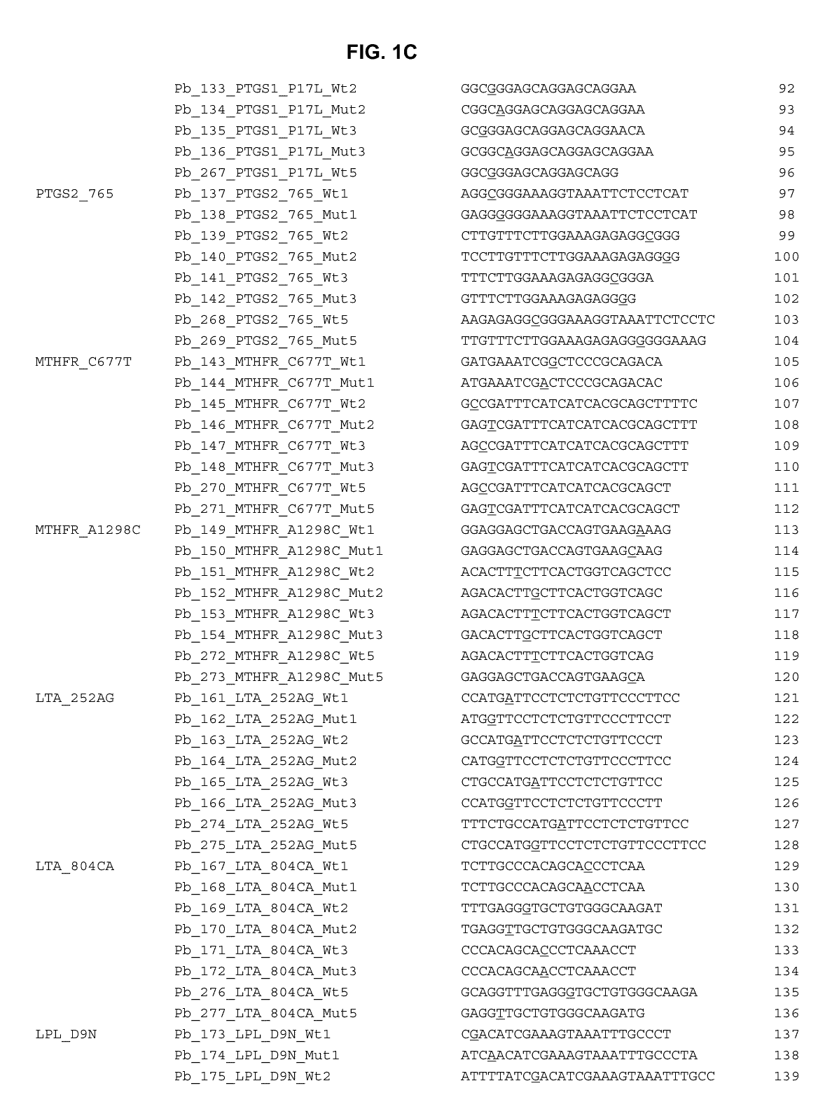

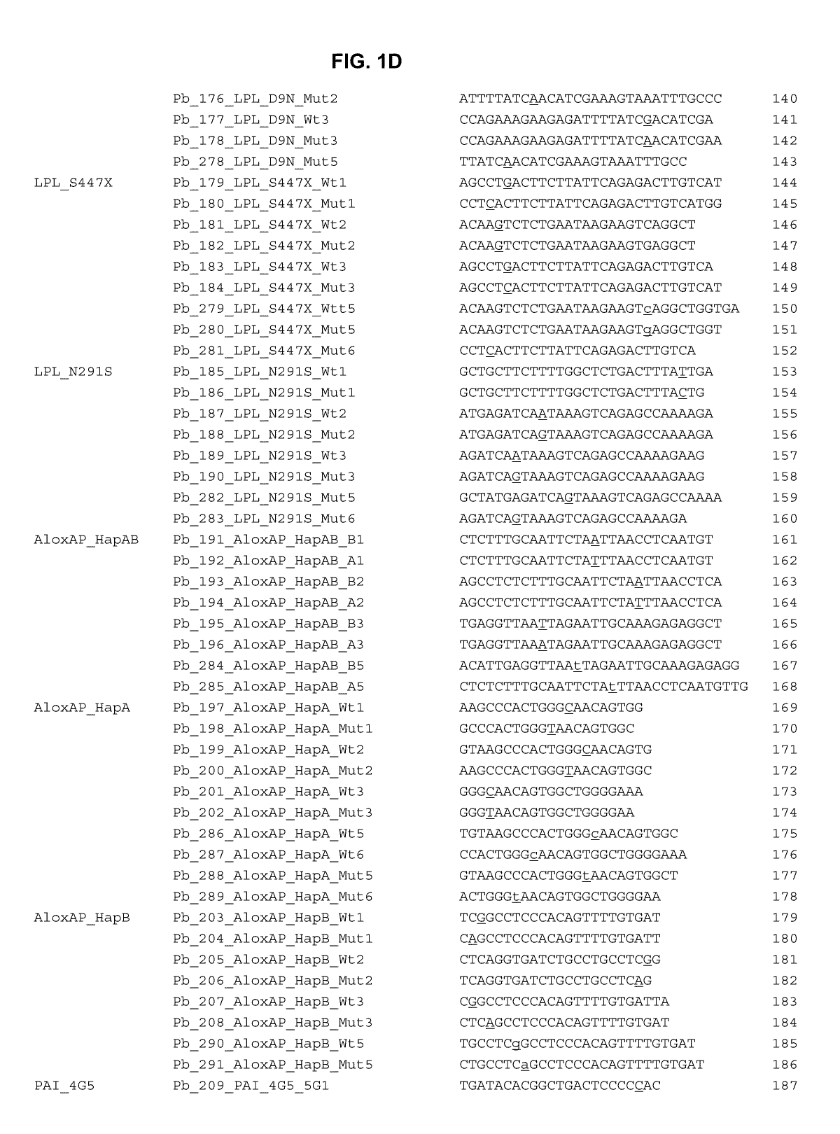

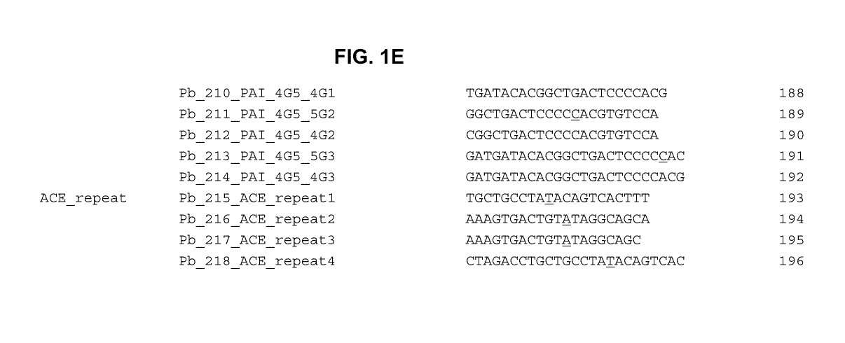

Also provided herein are probes that are extended or shortened versions of those disclosed in FIGS. 1A-1E or elsewhere herein. For example, a probe disclosed herein can be shortened by 1, 2, 3, 4, 5, or 6 nucleotides, on either or both ends. A probe disclosed herein can be extended by 1, 2, 3, 4, 5, 6 or more nucleotides on either or both ends. The extension can perfectly or substantially complementary to a region of a nucleic acid to which the probe binds before extension. Also provided herein are probes that are of the same length or substanially same length as other probes disclosed herein and that differ therefrom by 1, 2, 3, 4, 5 or 6 nucleotides.

In some embodiments, the length of a capture probe can be selected from about 10 to about 60 nucleic acids, about 10 to about 50 nucleic acids, about 10 to about 40 nucleic acids and about 10 to about 30 nucleic acids. In some embodiments, the length of a capture probe can be selected from about 15 to about 60 nucleic acids, about 15 to about 55 nucleic acids, about 15 to about 50 nucleic acids, about 15 to about 45 nucleic acids, about 15 to about 40 nucleic acids, about 15 to about 35 nucleic acids, and about 15 to about 30 nucleic acids. In an exemplary embodiment, the length of a capture probe is about 18 to about 33 nucleic acids. What is important is that the set of probes works well together in a multiplex assay as described herein.

Accordingly, the invention provides a capture probe set (e.g. used in an array comprising the capture probe set, each at a different location) that is used to determine whether a nucleic acid is characterized by any combination of the alleles in Table 1. In exemplary embodiments, determining whether a nucleic acid is characterized by an allele comprises determining the presence or absence of the allele in a target nucleic acid. As will be appreciated by those in the art, additional capture probes can be included, including negative and positive control sequences. In some embodiments, each of the alleles in Table 1 is probed. In some embodiments, only the alleles in Table 1 are probed. In some embodiments, a subset of the alleles in Table 1 is probed. In some embodiments, only a subset of the alleles in Table 1 is probed. The invention also provides a capture probe set for probing any combination of the alleles shown in Table 1.

In some embodiments, a capture probe set includes or excludes a capture probe that is selective for a G1006A allele of PTGS1. In some embodiments, a capture probe set includes or excludes a capture probe that is selective for a R8W allele of PTGS1. In some embodiments, a capture probe set includes or excludes a capture probe that is selective for a P17L allele of PTGS1. In some embodiments, a capture probe set includes or excludes a capture probe that is selective for a −765G/C allele of PTGS2. In some embodiments, a capture probe set includes or excludes a capture probe that is selective for a −786T/C allele of NOS3. In some embodiments, a capture probe set includes or excludes a capture probe that is selective for a E298D allele of NOS3. In some embodiments, a capture probe set includes or excludes a capture probe that is selective for a 4G/5G allele of SERPINE1. In some embodiments, a capture probe set includes or excludes a capture probe that is selective for a G1691A allele of F5. In some embodiments, a capture probe set includes or excludes a capture probe that is selective for a C677T allele of MTHFR. In some embodiments, a capture probe set includes or excludes a capture probe that is selective for a A1298C allele of MTHFR. In some embodiments, a capture probe set includes or excludes a capture probe that is selective for a HapAB allele of ALOX5AP. In some embodiments, a capture probe set includes or excludes a capture probe that is selective for a HapA allele of ALOX5AP. In some embodiments, a capture probe set includes or excludes a capture probe that is selective for a HapB allele of ALOX5AP. In some embodiments, a capture probe set includes or excludes a capture probe that is selective for a Taq1b allele of CETP. In some embodiments, a capture probe set includes or excludes a capture probe that is selective for a −629C/A allele of CETP. In some embodiments, a capture probe set includes or excludes a capture probe that is selective for a A1061G allele of CETP. In some embodiments, a capture probe set includes or excludes a capture probe that is selective for a A1163G allele of CETP. In some embodiments, a capture probe set includes or excludes a capture probe that is selective for a Cys112Arg allele of APOE. In some embodiments, a capture probe set includes or excludes a capture probe that is selective for a Arg158Cys allele of APOE. In some embodiments, a capture probe set includes or excludes a capture probe that is selective for a G20210A allele of F2. In some embodiments, a capture probe set includes or excludes a capture probe that is selective for a Ins/Del allele of ACE. In some embodiments, a capture probe set includes or excludes a capture probe that is selective for a 252A/G allele of LTA. In some embodiments, a capture probe set includes or excludes a capture probe that is selective for a 804C/A allele of LTA. In some embodiments, a capture probe set includes or excludes a capture probe that is selective for a D9N allele of LPL. In some embodiments, a capture probe set includes or excludes a capture probe that is selective for a S447X allele of LPL. In some embodiments, a capture probe set includes or excludes a capture probe that is selective for a N291S allele of LPL.

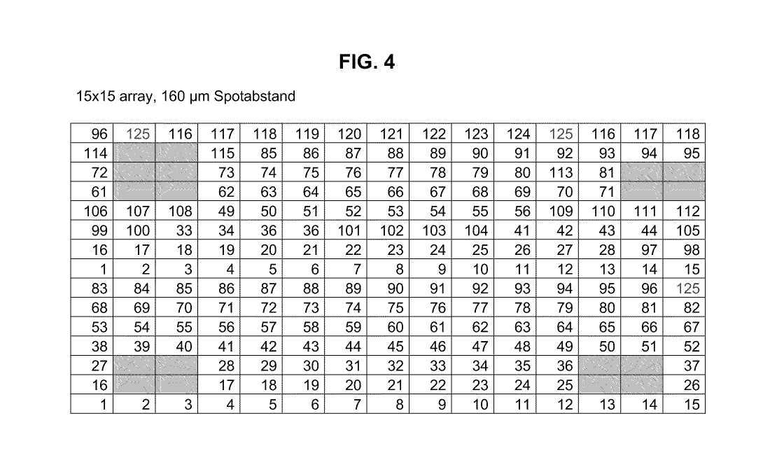

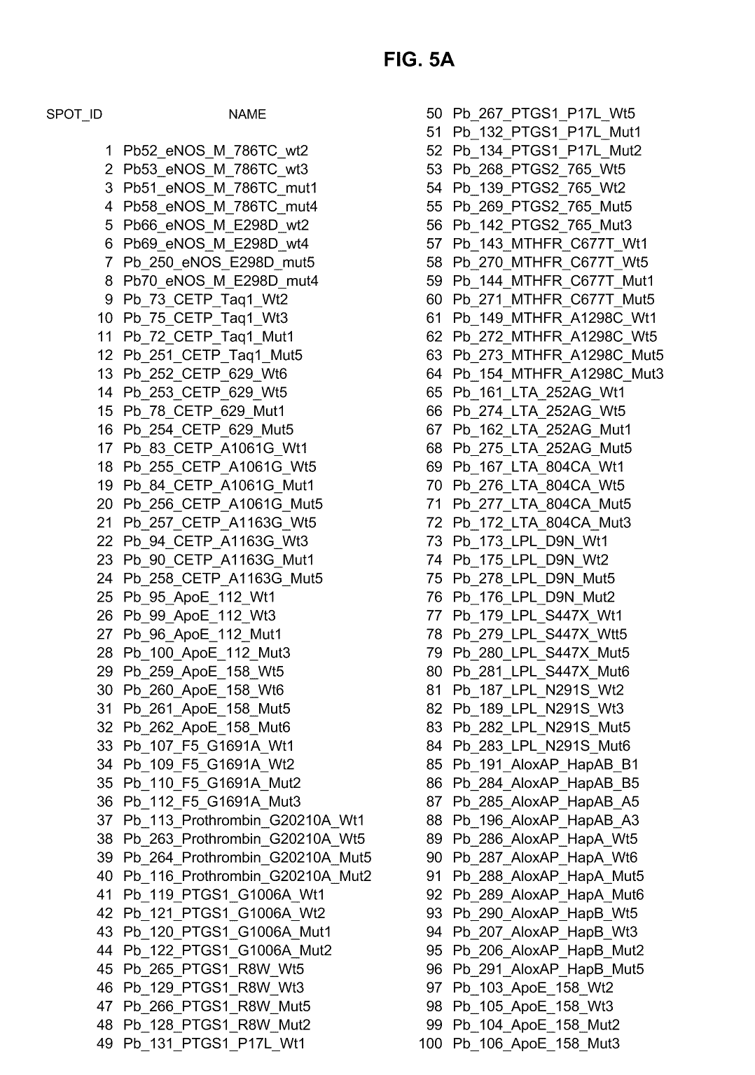

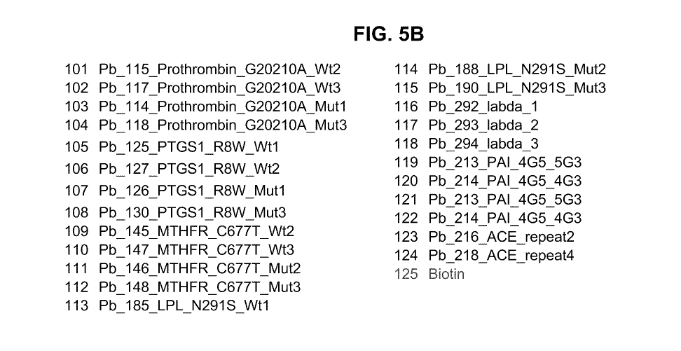

In some embodiments, a capture probe set comprises or consists of a combination of probes selected from FIGS. 1A-1E. In some embodiments, a capture probe set comprises or consists of a combination of probes selected from FIGS. 1A-1E. In some embodiments, a capture probe set comprises or consists of a combination of probes selected from FIG. 4. In some embodiments, a capture probe set comprises or consists of a combination of probes selected from FIGS. 5A-5B.

In some embodiments, a capture probe set consists of a plurality of nucleic acids having sequences according to SEQ ID NOS: 1, 6, 9, 11, 12, 13, 15, 16, 18, 20, 22, 27, 28, 29, 30, 31, 36, 37, 39, 43, 44, 45, 46, 47, 50, 51, 54, 55, 56, 57, 58, 59, 60, 61, 62, 64, 65, 67, 68, 69, 70, 71, 72, 73, 74, 75, 76, 77, 78, 79, 82, 83, 84, 85, 86, 87, 88, 89, 90, 91, 92, 96, 99, 102, 103, 104, 105, 106, 107, 108, 109, 110, 111, 112, 113, 118, 119, 120, 121, 122, 127, 128, 129, 134, 135, 136, 138, 139, 140, 143, 144, 150, 151, 152, 153, 155, 156, 157, 158, 159, 160, 161, 166, 167, 168, 175, 176, 177, 178, 182, 183, 185, 186, 191, 191, 192, 192, 194 and 196. In some embodiments, the above capture probe set further consists of a plurality of nucleic acids having sequences according to SEQ ID NOS: 2, 3 and 5, in any combination, for example, one selected from SEQ ID NOS: 2 and 3; 2 and 5; and 3 and 5.

### Primers

The invention also provides primers that are useful for genotyping a target sequence to determine disease risk or status. Additionally, primer sets are provided that include any combination of the primers disclosed herein. The kits described herein can comprise a primer set comprising any combination of the primers disclosed herein. Any primer can also be modified to hybridize to any gene (i.e. any allele) disclosed herein under stringent conditions, high stringency conditions or other appropriate conditions as known in the art.

In general, current methods for detecting gene variants utilize a first amplification step such as PCR to amplify sections of a nucleic acid, such as those comprising a gene. As will be appreciated by those in the art, small fragments of a gene can be amplified to allow more efficient and less expensive processing. In addition, a label or a detectable label is preferably added during the amplification step. The primers disclosed herein can be allowed to bind to a target sequence and can be extended using polymerases as known in the art.

Thus, in one embodiment, a target sequence comprises a detectable label, as described herein. A “label”, “detectable label” or “detectable marker” used interchangeably herein is an atom (such as an isotope) or molecule attached to a compound to enable the detection of the compound. In general, labels fall into four classes: a) isotopic labels, which may be radioactive or heavy isotopes; b) magnetic, electrical, thermal; c) colored or luminescent dyes; and d) enzymes, although labels include particles such as magnetic particles as well. The dyes may be chromophores or phosphors but in some exemplary embodiments are fluorescent dyes, which because of their strong signals provide a good signal-to-noise ratio for decoding. Suitable dyes for use in the invention include, but are not limited to, fluorescent lanthanide complexes, including those of europium and terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in Molecular Probes Handbook (6th ed.) by Richard P. Haugland. Additional labels include nanocrystals or Q-dots as described in U.S. Pat. No. 6,544,732.

A detectable label can be incorporated in a variety of ways for detection of a target sequence. In various embodiments, the target sequence is labeled; binding of the target sequence thus provides the label at the surface of the solid support. In various embodiments, a sandwich format is utilized, in which a target sequence is unlabeled. In these embodiments, a capture probe is attached to a detection surface as described herein, and a soluble binding ligand (also referred to as a “signaling probe,” “label probe” or “soluble capture ligand”) binds independently to the target sequence and either directly or indirectly comprises at least one label or detectable marker. A detectable label may refer to one or more components of a set of binding partners forming a binding complex. Thus, in various embodiments, a detectable label comprises (a) biotin, (b) biotin bound to streptavidin or (c) biotin bound to a streptavidin conjugate. In various embodiments, the detectable label comprises an enzyme (for example, horseradish peroxidase (HRP)). In various embodiments, the enzyme is a conjugated enzyme (for example, HRP-streptavidin). In various embodiments, the system relies on detecting the precipitation of a reaction product or on a change in, for example, electronic properties for detection. In various embodiments, none of the compounds comprises a label.

In exemplary embodiments, a detectable label is added to the target sequence during amplification of the target through the use of either labeled primers or labeled dNTPs, both of which are well known in the art. Labeled dNTPs could thus be incorporated during amplification. In some embodiments, each of the primers comprises a detectable label.

A detectable label can either be a primary or secondary label. A primary label produces a detectable signal that can be directly detected. For example, the label on a primer or a dNTP is a primary label such as a fluorophore. Alternatively, a label may be a secondary label, such as biotin or an enzyme. A secondary label requires additional reagents that lead to the production of a detectable signal. A secondary label is one that is indirectly detected; for example, a secondary label can bind or react with a primary label for detection, can act on an additional product to generate a primary label, or may allow the separation of the compound comprising the secondary label from unlabeled materials, etc. Secondary labels include, but are not limited to, one of a binding partner pair, such as biotin; chemically modifiable moieties; nuclease inhibitors; enzymes such as horseradish peroxidase; alkaline phosphatases; lucifierases, etc. Secondary labels can also include additional labels.

In some embodiments, the primers or dNTPs are labeled with biotin, which can then be contacted with a streptavidin/label complex. In some embodiments, the streptavidin/label complex comprises a fluorophore. In exemplary embodiments, the streptavidin/label complex comprises an enzymatic label. For example, the enzymatic label can be horseradish peroxidase, and upon contact with a precipitating agent, such as 3,3′,5,5′-tetramethylbenzidine (TMB) or o-dianisidine (3,3′-dimethoxybenzidine (dihydrochloride), Fast Blue B), an optically detectable precipitation reaction occurs. This has a particular benefit in that the optics for detection do not require the use of a fluorimeter or other detector, which can add to the expense of carrying out the methods.

In various embodiments, the secondary label is a binding partner pair. For example, the label may be a hapten or antigen, which will bind its binding partner. Suitable binding partner pairs include, but are not limited to: antigens (such as a polypeptide) and antibodies (including fragments thereof (FAbs, etc.)); other polypeptides and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents.

Primer pairs can be used to amplify an entire gene or shorter fragments of a gene, any of which are then used as the target sequences. Thus, a primer pair suitable for amplifying a gene is also suitable for amplifying a fragment of the gene. In some cases, a single amplicon may contain two or more SNP positions; alternatively, separate amplicons are generated for each SNP location. In some embodiments, a primer pair is used to amplify an entire gene or fragment of the gene, either of which contains a substitution, insertion or deletion relevative to another gene or gene fragment. For example, the amplified product could be used to determine the presence or absence of any of the variations shown in Table 1.

In some embodiments, one or more control primers are used. In various embodiments, any combination of the primers disclosed herein may be used.

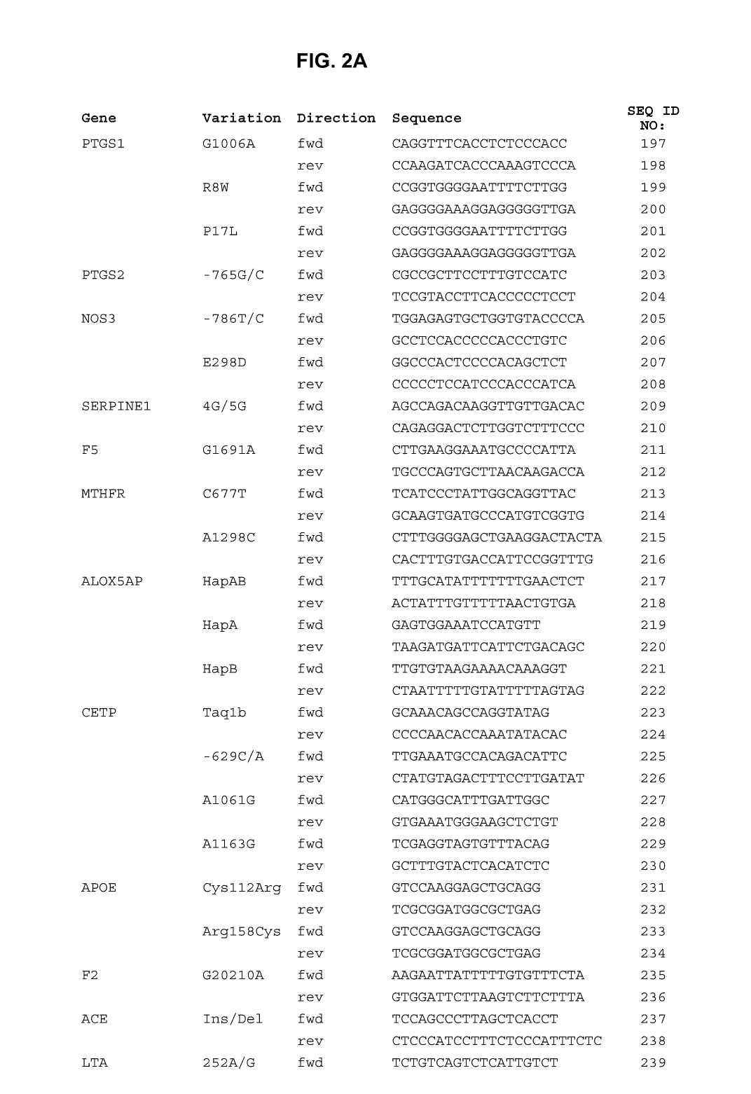

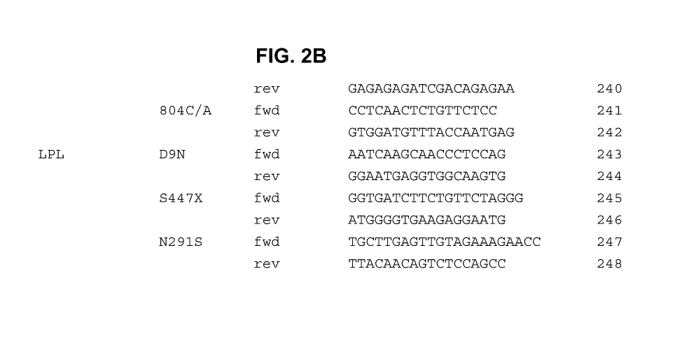

Exemplary primers that are useful in the kits, compositions and methods of the invention are shown in FIGS. 2A-2B. Each of the primers shown in FIGS. 2A-2B is considered suitable for generating an amplicon comprising a sequence or a portion of a sequence of the respective gene indicated. Methods for designing primers suitable for amplifying a gene are known in the art. See, for example, Innis M A, Gelfand D H, Sninsky J J, White T J (1990) PCR Protocols. A Guide to Methods and Applications. Academic Press, San Diego, Calif.

Also provided herein are primers that are extended or shortened versions of those disclosed in FIGS. 2A-2B or elsewhere herein. For example, a primer disclosed herein can be shortened by 1, 2, 3, 4, 5, or 6 nucleotides, on either or both ends. A primer disclosed herein can be extended by 1, 2, 3, 4, 5, 6 or more nucleotides on either or both ends. The extension can perfectly or substantially complementary to a region of a nucleic acid to which the primer binds before extension. Also provided herein are primers that are of the same length or substanially same length as the primers disclosed herein and that differ therefrom by 1, 2, 3, 4, 5 or 6 nucleotides.

As will be appreciated by those in the art, the length of a primer can vary. In some embodiments, the length of a primer is selected from about 10 to about 60 nucleic acids, about 10 to about 50 nucleic acids, about 10 to about 40 nucleic acids and about 10 to about 30 nucleic acids. In some embodiments, the length of a primer is selected from about 15 to about 60 nucleic acids, about 15 to about 55 nucleic acids, about 15 to about 50 nucleic acids, about 15 to about 45 nucleic acids, about 15 to about 40 nucleic acids, about 15 to about 35 nucleic acids, and about 15 to about 30 nucleic acids. In some embodiments, the length of a primer is about 18 to about 22 nucleic acids. In some embodiments, the length of a primer is about 17 to about 28 nucleic acids. In exemplary embodiments, a primer has a length of about 17 to about 25 nucleic acids. Any set of primers disclosed herein may also be used.

In some embodiments, a primer set comprises or consists of any combination of primers selected from those in FIGS. 2A-2B. In some embodiments, a primer set consists of the primers shown in FIGS. 2A-2B.

### Methods

The invention provides methods for characterizing alleles of various genes in a nucleic acid. Any method of the invention may be carried out using the various probes, primers, solid supports and kits described herein.

In one aspect, the invention provides a method of detecting a plurality of alleles in a nucleic acid, the method comprising: (a) generating a plurality of amplicons in a sample comprising the nucleic acid, wherein each of the plurality of amplicons comprises a detectable label; (b) contacting the plurality of amplicons with a solid support of the invention; and (c) detecting the presence or absence of the detectable label, thereby detecting one or more alleles (or a plurality of alleles) in the nucleic acid. In one embodiment, the generating step comprises contacting the sample with a primer set of the invention or with a primer set of a kit of the invention. The solid support can also be a solid support of a kit of the invention. The plurality of alleles are those associated with a disease, for example, atherosclerosis. In exemplary embodiments, the plurality of alleles is any combination of alleles, which alleles are disclosed herein. In these and other methods, the nucleic acid is typically one suspected of comprising one or more of the alleles being detected, for example, a target sequence derived from genomic DNA, mRNA, amplification products derived therefrom or any target sequence described herein.

In some embodiments, the generating step comprises using a DNA polymerase known in the art (e.g. Taq polymerase). In exemplary embodiments, the detecting step comprises causing precipitation of a precipitating agent. In exemplary embodiments, the detecting step comprises contacting the sample with a conjugated enzyme. Particularly useful conjugated enzymes include those can oxidize or reduce a precipitation agent. Examples include a horseradish peroxidase conjugate, for example, HRP-streptavidin or other conjugate disclosed herein or known in the art. In exemplary embodiments, the detecting step comprises contacting the sample with a precipitating agent, for example, o-dianisidine. In exemplary embodiments, the sample is derived from a subject experiencing or at risk of experiencing a disease, for example atherosclerosis.

In one aspect, the invention provides a method of assessing risk of disease (such as atherosclerosis) in a subject, the method comprising determining whether a nucleic acid in a sample from the subject is characterized by a plurality of gene variants associated with a disease or disease risk, such as atherosclerosis or atherosclerotic risk. In exemplary embodiments, the plurality of gene variants is selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

The plurality of gene variants can comprise or consist of any combination of these variants. For example, in one embodiment, the plurality of gene variants comprises a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL. In one embodiment, the plurality of gene variants comprises a combination of gene variants selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL. In one embodiment, the plurality of gene variants consists of a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL. In one embodiment, the plurality of gene variants consists of a combination of gene variants selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

A variant of gene can be any variant disclosed herein. Thus, in some embodiments, the variant of PTGS1 is selected from G1006A, R8W and P17L; the variant of PTGS2 is −765G/C; the variant of NOS3 is selected from −786T/C and E298D; the variant of SERPINE1 is 4G/5G; the variant of F5 is G1691A; the variant of MTHFR is selected from C677T and A1298C; the variant of ALOX5AP is selected from HapAB, HapA and HapB; the variant of CETP is selected from Taq1b, −629C/A, A1061G and A1163G; the variant of APOE is selected from C112R and R158C; the variant of F2 is selected from G20210A; the variant of ACE is ins/del; the variant of LTA is selected from 252A/G and 804C/A or the variant of LPL is selected from D9N, S447X and N291S.

In exemplary embodiments, the determining step comprises generating a plurality of amplicons in a sample comprising the nucleic acid, wherein the generating step comprises contacting the sample with a primer set comprising a plurality of primers suitable for amplifying the plurality of gene variants and wherein each of the plurality of amplicons comprises a detectable label; contacting the plurality of amplicons with a solid support comprising a plurality of capture probes selective for a plurality of variants associated with atherosclerotic risk (such as a combination selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL); and detecting the presence or absence of the detectable label. These variants are disclosed herein.

In some embodiments, the generating step comprises using a DNA polymerase known in the art (e.g. Taq polymerase). In exemplary embodiments, the detecting step comprises causing precipitation of a precipitating agent. In exemplary embodiments, the detecting step comprises contacting the sample with a conjugated enzyme. Particularly useful conjugated enzymes include those can oxidize or reduce a precipitation agent. Examples include a horseradish peroxidase conjugate, for example, HRP-streptavidin or other conjugate disclosed herein or known in the art. In exemplary embodiments, the detecting step comprises contacting the sample with a precipitating agent, for example, o-dianisidine. In exemplary embodiments, the sample is derived from a subject experiencing or at risk of experiencing a disease, for example atherosclerosis.

In some embodiments, it is implied that a nucleic acid is tested for the presence of all of these alleles or subset of these alleles. In some embodiments, it is understood that a nucleic acid that is being tested does not need to be finally determined to be characterized by all or any of these alleles. Any of the methods disclosed herein can be performed using the various kits, compositions, primer sets or probe sets disclosed herein.

### Procedure

### Amplification and Biotinylation

For the amplification of target DNA a special multiplex reaction was designed to be capable of amplifying all target fragments in five different tubes, reducing pipetting steps and thus labor time enormously. To accomplish this, TrueStart™ Hot Start Taq DNA polymerase from Fermentas (Fermentas International inc., Canada) is used. In a 25 μl reaction volume, 1×“10× True Start Taq buffer” is combined with 1 units of TrueStart™ Hot Start Taq DNA Polymerase (Fermentas), 0.2 mM dNTP mix (Mix includes 2 mM dATPs, dGTPs and dCTPs, 1.5 mM dTTPs and 0.5 mM 16-Bio-dUTPs from Roche Diagnostics International), 0.2 mM of each primer as listed in Table 1, 360 ng extracted DNA and 1 mM MgCl₂. Cycling conditions (using the MJ Research PTC-200 Peltier Thermal Cycler, Biozym Diagnostik GmbH, Oldendorf) were selected as follows: 2 min. initial denaturation at 95° C., 35 cycles of 30 s denaturation at 94° C., 1 min annealing at different temperatures (see table below), 1 min. elongation at 72° C., and a final elongation at 72° C. for 5 min.

Overview of the multiplex mixtures used for
amplification and biotynilation by PCR
(each Primer 5 pM/μl, additional 25 mM/μl MgCl₂)

| | Annealing | | |
|---|---|---|---|
| | Temperature | | |
| Tube | [° C.] | 0.5 μl of each primer: | 1 μl of each primer: |
| 1 | 64° C. | LPL N291S, MgCl₂ | |
| 2 | 60° C. | NOS3 −786T/C, MTHFR | LTA 804C/A & |
| | | A1298C, PTGS1 G1006A, | 252A/G, CETP |
| | | PTGS2 −765G/C, SERPINE1 | A1163G, Alox AP |
| | | 4G/5G, ACE ins/del | HapA & HapAB |
| 3 | 62° C. | F5 G1691A, MTHFR C677T, | PTGS1 R8W & |
| | | LPL S447X & D9N, CETP | P17L, CETP taq 1b |
| | | −629C/A & A1061G, MgCl₂ | |
| 4 | 62° C. | NOS3 E298D, LPL S447X | F2 G20210A, |
| | | | MgCl₂ |
| 5 | 60° C. | 2 μl DMSO | 3 μl ApoE |
| | | | Cys112Arg & |
| | | | Arg158Cys |

Biotinylation provides one useful means of labeling a target species. In addition to using biotinylated dUTP, other dNTPs in any combination may be biotinylated as well. Furthermore, the primers used in amplification of the target species may also be biotinylated.

Probes and primers targeting different mutations in genes, correlated to the development of atherosclerosis, were designed by calculation their specific melting temperatures by means of the algorithm according to SantaLucia, “A unified view of polymer, dumbbell, and oligonucleotide DNA nearest-neighbor thermodynamics”, Proc. Natl. Acad. Sci, USA, 1998, 95: 1460-1465, were blasted against each other and the genomic and target DNA, and were experimentally adjusted. The pattern of probes of the chip is shown in FIG. 4. Sequences of these probes are listed in FIGS. 1A-1E and referred to in FIGS. 5A-5B.

Generally, any method of DNA amplification as known in the art may be used. In various embodiments, DNA target species are amplified directly from whole blood. In various embodiments, the method of DNA amplification comprises isothermal amplification as known in the art.

### Hybridization Assay

Required solutions
Hybridization buffer
   3DNA buffer from Clondiag (Germany)
Wash buffer I
   2×SSC: mix 10 mL 20×SSC with 90 mL distilled water
   Mix 100 mL 2×SSC with 10 μL Triton X100
Wash buffer II
   Mix 10 mL 20×SSC with 90 mL distilled water
Wash buffer III
   Mix 1 mL 20×SSC with 99 mL distilled water
AT blocking solution
   Prepare 6×SSPE: Mix 30 mL 20×SSPE with 70 mL distilled water
   Then mix 100 mL 6×SSPE+5 μL Triton X100 and 0.02 g blocking reagent (Roche)
Poly horseradish peroxidase conjugate (HRP conjugate) solution
   Mix 1.5 mL 20×SSPE with 3.5 mL distilled water
   Add 1 μL POLY HRP enzyme (Thermo Fisher Scientific, USA)
O-dianizidine substrate solution (Seramun Diagnostica, Germany)

### Process

The macroarray chip is initially conditioned with 500 μL distilled water. In the second step the chip is conditioned with 200 μl hybridization buffer at 550 rpm (Eppendorf thermomixer compact) and 50° C. for 2 min. Next the biotinylated product is heated up to 95° C. for 2 min. and mixed with 90 μl hybridization buffer. This blend is then incubated on the chip for 45 min, at 550 rpm and 50° C. After hybridization, the chip is washed three times with the washing buffers I, II and III, respectively, with 500 μl each for 5 min and at 50° C., 50° C. and 50° C., respectively. Upon washing, blocking solution is freshly prepared and 100 μl is incubated on the chip for 15 min at 550 rpm and room temperature (RT). The next step comprises the addition of 100 μl freshly prepared HRP conjugate solution and incubation for 15 min at 550 rpm and RT. Following this, the unbound conjugate is washed away by adding washing buffers I, II and III in sequence as described above. The precipitation reaction is introduced by adding 100 μl of O-dianizidine substrate. After 5 min. the results can be read out. Cutoff values for genotyping are about 0.3 for a positive signal, if the Clondiag software IconoClust is used for read out.

### Examples

### Example 1

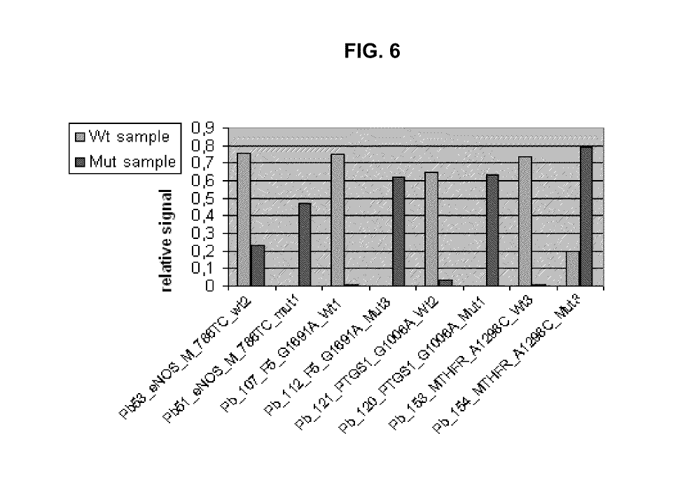

FIGS. 4 and 5A-5B show a number of probes that were attached to a solid support to produce a biochip for assaying a sample. Protocols described above were performed, and typical results for selected probes are shown in FIG. 6.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-exhaustive examples.

All references, publications, patent applications, issued patents, accession records and databases cited herein, including in any appendices, are incorporated by reference in their entirety for all purposes.

### CITATIONS

1. Asensi V, Montes A H, Valle E, Ocaña M G, Astudillo A, Alvarez V, López-Anglada E, Solis A, Coto E, Meana A, Gonzalez P, Carton J A, Paz J, Fierer J, Celada A 2006. The NOS3 (27-bp repeat, intron 4) polymorphism is associated with susceptibility to osteomyelitis. Am J Epidemiol. 164: 921-935
2. Bertina R M, Koeleman B P, Koster T, Rosendaal F R, Dirven R J, de Ronde H, van der Velden P A, Reitsma P H 1994. Mutation in blood coagulation factor V associated with resistance to activated protein C. Nature. 369: 64-67
3. Kara I, Sazci A, Ergul E, Kaya G, Kilic G 2003. Association of the C677T and A1298C polymorphisms in the 5,10 methylenetetrahydrofolate reductase gene in patients with migraine risk. Brain Res Mol Brain Res. 111: 84-90
4. Koeleman B P, Reitsma P H, Allaart C F, Bertina R M 1994. Activated protein C resistance as an additional risk factor for thrombosis in protein C-deficient families. Blood. 84: 1031-1035
5. Nauck M, Wieland H, März W 1999. Rapid, homogeneous genotyping of the 4G/5G polymorphism in the promoter region of the PAII gene by fluorescence resonance energy transfer and probe melting curves. Clin Chem. 45: 1141-1147
6. Rigat B, Hubert C, Corvol P, Soubrier F 1992. PCR detection of the insertion/deletion polymorphism of the human angiotensin converting enzyme gene (DCP1) (dipeptidyl carboxypeptidase 1). Nucleic Acids Res. 20: 1433

1. A kit comprising a solid support comprising a capture probe set comprising a plurality of probes selected from (a) a probe selective for PTGS1, (b) a probe selective for PTGS2, (c) a probe selective for NOS3, (d) a probe selective for SERPINE1, (e) a probe selective for F5, (f) a probe selective for MTHFR, (g) a probe selective for ALOX5AP, (h) a probe selective for CETP, (i) a probe selective for APOE, (j) a probe selective for F2, (k) a probe selective for ACE, (l) a probe selective for LTA and (m) a probe selective for LPL.

2. The kit of claim 1 wherein the capture probe set comprises (a) a probe selective for a G1006A allele of PTGS1, (b) a probe selective for a R8W allele of PTGS1, (c) a probe selective for a P17L allele of PTGS1, (d) a probe selective for a −765G/C allele of PTGS2, (e) a probe selective for a −786T/C allele of NOS3, (f) a probe selective for a E298D allele of NOS3, (g) a probe selective for a 4G/5G allele of SERPINE1, (h) a probe selective for a G1691A allele of F5, (i) a probe selective for a C677T allele of MTHFR, (j) a probe selective for a A1298C allele of MTHFR, (k) a probe selective for a HapAB allele of ALOX5AP, (l) a probe selective for a HapA allele of ALOX5AP, (m) a probe selective for a HapB allele of ALOX5AP, (n) a probe selective for a Taq1b allele of CETP, (o) a probe selective for a −629C/A allele of CETP, (p) a probe selective for a A1061G allele of CETP, (q) a probe selective for a A1163G allele of CETP, (r) a probe selective for a Cys112Arg allele of APOE, (s) a probe selective for a Arg158Cys allele of APOE, (t) a probe selective for a G20210A allele of F2, (u) a probe selective for a Ins/Del allele of ACE, (v) a probe selective for a 252A/G allele of LTA, (w) a probe selective for a 804C/A allele of LTA, (x) a probe selective for a D9N allele of LPL, (y) a probe selective for a S447X allele of LPL, and (z) a probe selective for a N291S allele of LPL.

3. The kit of claim 1 wherein the capture probe set comprises (a) (i) a probe selective for a first G1006A allele of PTGS1 and (ii) a probe selective for a second G1006A allele of PTGS1; (b) (i) a probe selective for a first R8W allele of PTGS1 and (ii) a probe selective for a second R8W allele of PTGS1; (c) (i) a probe selective for a first P17L allele of PTGS1 and (ii) a probe selective for a second P17L allele of PTGS 1; (d) (i) a probe selective for a first −765G/C allele of PTGS2 and (ii) a probe selective for a second −765G/C allele of PTGS2; (e) (i) a probe selective for a first −786T/C allele of NOS3 and (ii) a probe selective for a second −786T/C allele of NOS3; (f) (i) a probe selective for a first E298D allele of NOS3 and (ii) a probe selective for a second E298D allele of NOS3; (g) (i) a probe selective for a first 4G/5G allele of SERPINE1 and (ii) a probe selective for a second 4G/5G allele of SERPINE1; (h) (i) a probe selective for a first G1691A allele of F5 and (ii) a probe selective for a second G1691A allele of F5; (i) (i) a probe selective for a first C677T allele of MTHFR and (ii) a probe selective for a second C677T allele of MTHFR; (j) (i) a probe selective for a first Al298C allele of MTHFR and (ii) a probe selective for a second A1298C allele of MTHFR; (k) (i) a probe selective for a first HapAB allele of ALOX5AP and (ii) a probe selective for a second HapAB allele of ALOX5AP; (1) (i) a probe selective for a first HapA allele of ALOX5AP and (ii) a probe selective for a second HapA allele of ALOX5AP; (m) (i) a probe selective for a first HapB allele of ALOX5AP and (ii) a probe selective for a second HapB allele of ALOX5AP; (n) (i) a probe selective for a first Taq1b allele of CETP and (ii) a probe selective for a second Taq1b allele of CETP; (o) (i) a probe selective for a first −629C/A allele of CETP and (ii) a probe selective for a second −629C/A allele of CETP; (p) (i) a probe selective for a first A1061G allele of CETP and (ii) a probe selective for a second A1061G allele of CETP; (q) (i) a probe selective for a first A1163G allele of CETP and (ii) a probe selective for a second A1163G allele of CETP; (r) (i) a probe selective for a first Cys112Arg allele of APOE and (ii) a probe selective for a second Cys112Arg allele of APOE; (s) (i) a probe selective for a first Arg158Cys allele of APOE and (ii) a probe selective for a second Arg158Cys allele of APOE; (t) (i) a probe selective for a first G20210A allele of F2 and (ii) a probe selective for a second G20210A allele of F2; (u) (i) a probe selective for a first Ins/Del allele of ACE and (ii) a probe selective for a second Ins/Del allele of ACE; (v) (i) a probe selective for a first 252A/G allele of LTA and (ii) a probe selective for a second 252A/G allele of LTA; (w) (i) a probe selective for a first 804C/A allele of LTA and (ii) a probe selective for a second 804C/A allele of LTA; (x) (i) a probe selective for a first D9N allele of LPL and (ii) a probe selective for a second D9N allele of LPL; (y) (i) a probe selective for a first S447X allele of LPL and (ii) a probe selective for a second S447X allele of LPL; and (z) (i) a probe selective for a first N291S allele of LPL and (ii) a probe selective for a second N291S allele of LPL.

4. The kit of claim 1 wherein each of the probes is an isolated nucleic acid comprising a sequence selected from SEQ ID NOS: 1-196 or its complement, wherein each of the isolated nucleic acids is characterized by a length of about 18 to about 50 nucleic acids.

5. The kit of claim 1 wherein each of the probes is an isolated nucleic acid consisting of a sequence selected from SEQ ID NOS: 1-196 or its complement.

6. The kit of claim 1 wherein the capture probe set consists of a plurality of nucleic acids having sequences according to SEQ ID NOS: 1, 6, 9, 11, 12, 13, 15, 16, 18, 20, 22, 27, 28, 29, 30, 31, 36, 37, 39, 43, 44, 45, 46, 47, 50, 51, 54, 55, 56, 57, 58, 59, 60, 61, 62, 64, 65, 67, 68, 69, 70, 71, 72, 73, 74, 75, 76, 77, 78, 79, 82, 83, 84, 85, 86, 87, 88, 89, 90, 91, 92, 96, 99, 102, 103, 104, 105, 106, 107, 108, 109, 110, 111, 112, 113, 118, 119, 120, 121, 122, 127, 128, 129, 134, 135, 136, 138, 139, 140, 143, 144, 150, 151, 152, 153, 155, 156, 157, 158, 159, 160, 161, 166, 167, 168, 175, 176, 177, 178, 182, 183, 185, 186, 191, 191, 192, 192, 194, 196 and a combination selected from SEQ ID NOS: 2 and 3; 2 and 5; and 3 and 5.

7. The kit of claim 1 further comprising a primer set comprising a plurality of primers selected from (a) a primer suitable for amplifying PTGS1, (b) a primer suitable for amplifying PTGS2, (c) a primer suitable for amplifying NOS3, (d) a primer suitable for amplifying SERPINE1, (e) a primer suitable for amplifying F5, (f) a primer suitable for amplifying MTHFR, (g) a primer suitable for amplifying ALOX5AP, (h) a primer suitable for amplifying CETP, (i) a primer suitable for amplifying APOE, (j) a primer suitable for amplifying F2, (k) a primer suitable for amplifying ACE, (l) a primer suitable for amplifying LTA and (m) a primer suitable for amplifying LPL.

8. The kit of claim 7 wherein the primer set comprises a plurality of primer pairs selected from (a) a primer pair suitable for amplifying PTGS1, (b) a primer pair suitable for amplifying PTGS2, (c) a primer pair suitable for amplifying NOS3, (d) a primer pair suitable for amplifying SERPINE1, (e) a primer pair suitable for amplifying F5, (f) a primer pair suitable for amplifying MTHFR, (g) a primer pair suitable for amplifying ALOX5AP, (h) a primer pair suitable for amplifying CETP, (i) a primer pair suitable for amplifying APOE, (j) a primer pair suitable for amplifying F2, (k) a primer pair suitable for amplifying ACE, (l) a primer pair suitable for amplifying LTA and (m) a primer pair suitable for amplifying LPL.

9. The kit of claim 7 wherein each of the primers is an isolated nucleic acid comprising a sequence selected from SEQ ID NOS: 197-248 or its complement, wherein each of the isolated nucleic acids is characterized by a length of about 17 to about 50 nucleic acids.

10. The kit of claim 7 wherein each of the primers is an isolated nucleic acid consisting of a sequence selected from SEQ ID NOS: 197-248 or its complement.

11. The kit of claim 7 wherein at least one of the plurality of primers comprises a detectable label.

12. The kit of claim 11 wherein the detectable label is biotin.

13. The kit of claim 12 further comprising a conjugated enzyme.

14. The kit of claim 13 further comprising a precipitating agent.

15. A method of detecting a plurality of alleles in a nucleic acid, the method comprising:
(a) generating a plurality of amplicons in a sample comprising the nucleic acid, wherein the generating step comprises contacting the sample with a primer set comprising a plurality of primers selected from (a) a primer suitable for amplifying PTGS1, (b) a primer suitable for amplifying PTGS2, (c) a primer suitable for amplifying NOS3, (d) a primer suitable for amplifying SERPINE1, (e) a primer suitable for amplifying F5, (f) a primer suitable for amplifying MTHFR, (g) a primer suitable for amplifying ALOX5AP, (h) a primer suitable for amplifying CETP, (i) a primer suitable for amplifying APOE, (j) a primer suitable for amplifying F2, (k) a primer suitable for amplifying ACE, (l) a primer suitable for amplifying LTA and (m) a primer suitable for amplifying LPL and wherein each of the plurality of amplicons comprises a detectable label;
(b) contacting the plurality of amplicons with the solid support of the kit of claim 1; and
(c) detecting the presence or absence of the detectable label, thereby detecting the plurality of alleles in the nucleic acid.

16. The method of claim 15 wherein the detecting step comprises contacting the sample with a conjugated enzyme.

17. The method of claim 16 wherein the detecting step comprising contacting the sample with a precipitating agent.

18. The method of claim 15 wherein the sample is derived from a subject experiencing or at risk of experiencing atherosclerosis.

19. A method of assessing risk of atherosclerosis in a subject comprising: determining whether a nucleic acid in a sample from the subject is characterized by a plurality of gene variants selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

20. The method of claim 19 wherein the plurality of gene variants comprises a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

21. The method of claim 20 wherein the plurality of gene variants consists of a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL.

22. The method of claim 19 wherein the variant of PTGS1 is selected from G1006A, R8W and P17L; the variant of PTGS2 is −765G/C; the variant of NOS3 is selected from −786T/C and E298D; the variant of SERPINE1 is 4G/5G; the variant of F5 is G1691A; the variant of MTHFR is selected from C677T and Al298C; the variant of ALOX5AP is selected from HapAB, HapA and HapB; the variant of CETP is selected from Taq1b, −629C/A, A1061G and A1163G; the variant of APOE is selected from C112R and R158C; the variant of F2 is selected from G20210A; the variant of ACE is ins/del; the variant of LTA is selected from 252A/G and 804C/A or the variant of LPL is selected from D9N, S447X and N291S.

23. The method of claim 19 wherein the determining step comprises:
generating a plurality of amplicons in a sample comprising the nucleic acid, wherein the generating step comprises contacting the sample with a primer set comprising a plurality of primers suitable for amplifying the plurality of gene variants and wherein each of the plurality of amplicons comprises a detectable label;
contacting the plurality of amplicons with a solid support comprising a plurality of capture probes selective for a plurality of variants selected from a variant of PTGS1, a variant of PTGS2, a variant of NOS3, a variant of SERPINE1, a variant of F5, a variant of MTHFR, a variant of ALOX5AP, a variant of CETP, a variant of APOE, a variant of F2, a variant of ACE, a variant of LTA and a variant of LPL; and
detecting the presence or absence of the detectable label.


SEQUENCE LISTING (277 sequences)

SEQ ID NO: 1 - DNA, 20 bp, Homo Sapiens
agctcttccctggccggctg

SEQ ID NO: 2 - DNA, 21 bp, Homo Sapiens
aagctcttccctggctggctg

SEQ ID NO: 3 - DNA, 22 bp, Homo Sapiens
cagccagccagggaagagcttg

SEQ ID NO: 4 - DNA, 22 bp, Homo Sapiens
cagccggccagggaagagcttg

SEQ ID NO: 5 - DNA, 23 bp, Homo Sapiens
tcagccagccagggaagagcttg

SEQ ID NO: 6 - DNA, 23 bp, Homo Sapiens
tcaagctcttccctggctggctg

SEQ ID NO: 7 - DNA, 18 bp, Homo Sapiens
ggggatcatctggggcct

SEQ ID NO: 8 - DNA, 19 bp, Homo Sapiens
ggggatcatctggggcctg

SEQ ID NO: 9 - DNA, 19 bp, Homo Sapiens
ggggctcatctggggcctg

SEQ ID NO: 10 - DNA, 21 bp, Homo Sapiens
ctgcaggccccagatgagccc

SEQ ID NO: 11 - DNA, 20 bp, Homo Sapiens
ggggctcatctggggcctgc

SEQ ID NO: 12 - DNA, 21 bp, Homo Sapiens
ggggatcatctggggcctgca

SEQ ID NO: 13 - DNA, 24 bp, Homo Sapiens
gttctgggggatcatctggggcct

SEQ ID NO: 14 - DNA, 23 bp, Homo Sapiens
gaaccctaactcgaaccccagtg

SEQ ID NO: 15 - DNA, 23 bp, Homo Sapiens
ctgaaccctaacttgaaccccag

SEQ ID NO: 16 - DNA, 26 bp, Homo Sapiens
ctggggttcgagttagggttcagatc

SEQ ID NO: 17 - DNA, 27 bp, Homo Sapiens
actggggttcaagttagggttcagatc

SEQ ID NO: 18 - DNA, 24 bp, Homo Sapiens
actggggttcgagttagggttcag

SEQ ID NO: 19 - DNA, 24 bp, Homo Sapiens
cactggggttcaagttagggttca

SEQ ID NO: 20 - DNA, 25 bp, Homo Sapiens
ctggggttcaagttagggttcagat

SEQ ID NO: 21 - DNA, 22 bp, Homo Sapiens
ctctgggggggtatacagcctc

SEQ ID NO: 22 - DNA, 24 bp, Homo Sapiens
aactctgggtgggtatacagcctc

SEQ ID NO: 23 - DNA, 21 bp, Homo Sapiens
ggggtatacagcctctgagat

SEQ ID NO: 24 - DNA, 23 bp, Homo Sapiens
gggtgggtatacagcctctgaga

SEQ ID NO: 25 - DNA, 22 bp, Homo Sapiens
gaggctgtatacccccccagag

SEQ ID NO: 26 - DNA, 24 bp, Homo Sapiens
cagaggctgtatacccacccagag

SEQ ID NO: 27 - DNA, 20 bp, Homo Sapiens
tctgggggggtatacagcct

SEQ ID NO: 28 - DNA, 28 bp, Homo Sapiens
ctctgggggggtatacagcctctgagat

SEQ ID NO: 29 - DNA, 28 bp, Homo Sapiens
actctgggtgggtatacagcctctgaga

SEQ ID NO: 30 - DNA, 23 bp, Homo Sapiens
aggaagctctggatggactcgga

SEQ ID NO: 31 - DNA, 22 bp, Homo Sapiens
ggaagctctggacggactcgga

SEQ ID NO: 32 - DNA, 21 bp, Homo Sapiens
ctctggacggactcggagctg

SEQ ID NO: 33 - DNA, 22 bp, Homo Sapiens
ctctggatggactcggagctgc

SEQ ID NO: 34 - DNA, 23 bp, Homo Sapiens
ctccgagtccatccagagcttcc

SEQ ID NO: 35 - DNA, 23 bp, Homo Sapiens
ctccgagtccgtccagagcttcc

SEQ ID NO: 36 - DNA, 27 bp, Homo Sapiens
agctccgagtccatccagagcttcctg

SEQ ID NO: 37 - DNA, 26 bp, Homo Sapiens
agctctggacggactcggagctgctc

SEQ ID NO: 38 - DNA, 29 bp, Homo Sapiens
cctcttcgacatcatcaaccctgagatta

SEQ ID NO: 39 - DNA, 29 bp, Homo Sapiens
cttcggcatcatcaaccctgagattatca

SEQ ID NO: 40 - DNA, 25 bp, Homo Sapiens
cggcatcatcaaccctgagattatc

SEQ ID NO: 41 - DNA, 28 bp, Homo Sapiens
cttcgacatcatcaaccctgagattatc

SEQ ID NO: 42 - DNA, 22 bp, Homo Sapiens
cagggttgatgatgccgaagag

SEQ ID NO: 43 - DNA, 23 bp, Homo Sapiens
agggttgatgatgtcgaagaggc

SEQ ID NO: 44 - DNA, 30 bp, Homo Sapiens
cctcttcgacatcatcaaccctgagattat

SEQ ID NO: 45 - DNA, 30 bp, Homo Sapiens
ctcttcggcatcatcaaccctgagattatc

SEQ ID NO: 46 - DNA, 20 bp, Homo Sapiens
gacatggaggacgtgtgcgg

SEQ ID NO: 47 - DNA, 20 bp, Homo Sapiens
ggacatggaggacgtgcgcg

SEQ ID NO: 48 - DNA, 21 bp, Homo Sapiens
ccgcacacgtcctccatgtcc

SEQ ID NO: 49 - DNA, 20 bp, Homo Sapiens
cgcgcacgtcctccatgtcc

SEQ ID NO: 50 - DNA, 20 bp, Homo Sapiens
catggaggacgtgtgcggcc

SEQ ID NO: 51 - DNA, 20 bp, Homo Sapiens
gacatggaggacgtgcgcgg

SEQ ID NO: 52 - DNA, 22 bp, Homo Sapiens
ccaggcacttctgcaggtcatc

SEQ ID NO: 53 - DNA, 20 bp, Homo Sapiens
aggcgcttctgcaggtcatc

SEQ ID NO: 54 - DNA, 22 bp, Homo Sapiens
caggcacttctgcaggtcatcg

SEQ ID NO: 55 - DNA, 21 bp, Homo Sapiens
aggcgcttctgcaggtcatcg

SEQ ID NO: 56 - DNA, 24 bp, Homo Sapiens
gtacactgccaggcacttctgcag

SEQ ID NO: 57 - DNA, 22 bp, Homo Sapiens
gtacactgccaggcgcttctgc

SEQ ID NO: 58 - DNA, 27 bp, Homo Sapiens
actgccaggcacttctgcaggtcatcg

SEQ ID NO: 59 - DNA, 28 bp, Homo Sapiens
gtacactgccaggcacttctgcaggtca

SEQ ID NO: 60 - DNA, 27 bp, Homo Sapiens
actgccaggcgcttctgcaggtcatcg

SEQ ID NO: 61 - DNA, 29 bp, Homo Sapiens
gtacactgccaggcgcttctgcaggtcat

SEQ ID NO: 62 - DNA, 22 bp, Homo Sapiens
ctggacaggcgaggaatacagg

SEQ ID NO: 63 - DNA, 23 bp, Homo Sapiens
cctggacaggcaaggaatacagg

SEQ ID NO: 64 - DNA, 23 bp, Homo Sapiens
ggacaggcgaggaatacaggtat

SEQ ID NO: 65 - DNA, 24 bp, Homo Sapiens
cctggacaggcaaggaatacaggt

SEQ ID NO: 66 - DNA, 23 bp, Homo Sapiens
ctggacaggcgaggaatacaggt

SEQ ID NO: 67 - DNA, 25 bp, Homo Sapiens
ctggacaggcaaggaatacaggtat

SEQ ID NO: 68 - DNA, 26 bp, Homo Sapiens
ccaataaaagtgactctcagcgagcc

SEQ ID NO: 69 - DNA, 28 bp, Homo Sapiens
ccaataaaagtgactctcagcaagcctc

SEQ ID NO: 70 - DNA, 23 bp, Homo Sapiens
aagtgactctcagcgagcctcaa

SEQ ID NO: 71 - DNA, 23 bp, Homo Sapiens
aagtgactctcagcaagcctcaa

SEQ ID NO: 72 - DNA, 23 bp, Homo Sapiens
ctctcagcgagcctcaatgctcc

SEQ ID NO: 73 - DNA, 23 bp, Homo Sapiens
ctctcagcaagcctcaatgctcc

SEQ ID NO: 74 - DNA, 21 bp, Homo Sapiens
agtgactctcagcgagcctca

SEQ ID NO: 75 - DNA, 26 bp, Homo Sapiens
caataaaagtgactctcagcaagcct

SEQ ID NO: 76 - DNA, 21 bp, Homo Sapiens
cggcaggagagctcagaggtt

SEQ ID NO: 77 - DNA, 21 bp, Homo Sapiens
ccggcaggagagttcagaggt

SEQ ID NO: 78 - DNA, 22 bp, Homo Sapiens
cggcaggagagctcagaggttc

SEQ ID NO: 79 - DNA, 23 bp, Homo Sapiens
ccggcaggagagttcagaggttc

SEQ ID NO: 80 - DNA, 23 bp, Homo Sapiens
gaacctctgagctctcctgccgg

SEQ ID NO: 81 - DNA, 24 bp, Homo Sapiens
aagaacctctgaactctcctgccg

SEQ ID NO: 82 - DNA, 23 bp, Homo Sapiens
gcaagaaccggagcaagagactc

SEQ ID NO: 83 - DNA, 24 bp, Homo Sapiens
gcaagaaccagagcaagagactcc

SEQ ID NO: 84 - DNA, 24 bp, Homo Sapiens
gagtctcttgctccggttcttgct

SEQ ID NO: 85 - DNA, 25 bp, Homo Sapiens
ggagtctcttgctctggttcttgct

SEQ ID NO: 86 - DNA, 20 bp, Homo Sapiens
cttgctccggttcttgctgt

SEQ ID NO: 87 - DNA, 21 bp, Homo Sapiens
cttgctctggttcttgctgtt

SEQ ID NO: 88 - DNA, 28 bp, Homo Sapiens
ggagtctcttgctccggttcttgctgtt

SEQ ID NO: 89 - DNA, 22 bp, Homo Sapiens
caagaaccagagcaagagactc

SEQ ID NO: 90 - DNA, 20 bp, Homo Sapiens
gttcctgctcctgctcccgc

SEQ ID NO: 91 - DNA, 20 bp, Homo Sapiens
gttcctgctcctgctcctgc

SEQ ID NO: 92 - DNA, 20 bp, Homo Sapiens
ggcgggagcaggagcaggaa

SEQ ID NO: 93 - DNA, 21 bp, Homo Sapiens
cggcaggagcaggagcaggaa

SEQ ID NO: 94 - DNA, 21 bp, Homo Sapiens
gcgggagcaggagcaggaaca

SEQ ID NO: 95 - DNA, 22 bp, Homo Sapiens
gcggcaggagcaggagcaggaa

SEQ ID NO: 96 - DNA, 18 bp, Homo Sapiens
ggcgggagcaggagcagg

SEQ ID NO: 97 - DNA, 26 bp, Homo Sapiens
aggcgggaaaggtaaattctcctcat

SEQ ID NO: 98 - DNA, 27 bp, Homo Sapiens
gaggggggaaaggtaaattctcctcat

SEQ ID NO: 99 - DNA, 25 bp, Homo Sapiens
cttgtttcttggaaagagaggcggg

SEQ ID NO: 100 - DNA, 25 bp, Homo Sapiens
tccttgtttcttggaaagagagggg

SEQ ID NO: 101 - DNA, 22 bp, Homo Sapiens
tttcttggaaagagaggcggga

SEQ ID NO: 102 - DNA, 20 bp, Homo Sapiens
gtttcttggaaagagagggg

SEQ ID NO: 103 - DNA, 29 bp, Homo Sapiens
aagagaggcgggaaaggtaaattctcctc

SEQ ID NO: 104 - DNA, 28 bp, Homo Sapiens
ttgtttcttggaaagagaggggggaaag

SEQ ID NO: 105 - DNA, 23 bp, Homo Sapiens
gatgaaatcggctcccgcagaca

SEQ ID NO: 106 - DNA, 23 bp, Homo Sapiens
atgaaatcgactcccgcagacac

SEQ ID NO: 107 - DNA, 27 bp, Homo Sapiens
gccgatttcatcatcacgcagcttttc

SEQ ID NO: 108 - DNA, 27 bp, Homo Sapiens
gagtcgatttcatcatcacgcagcttt

SEQ ID NO: 109 - DNA, 26 bp, Homo Sapiens
agccgatttcatcatcacgcagcttt

SEQ ID NO: 110 - DNA, 26 bp, Homo Sapiens
gagtcgatttcatcatcacgcagctt

SEQ ID NO: 111 - DNA, 24 bp, Homo Sapiens
agccgatttcatcatcacgcagct

SEQ ID NO: 112 - DNA, 25 bp, Homo Sapiens
gagtcgatttcatcatcacgcagct

SEQ ID NO: 113 - DNA, 24 bp, Homo Sapiens
ggaggagctgaccagtgaagaaag

SEQ ID NO: 114 - DNA, 23 bp, Homo Sapiens
gaggagctgaccagtgaagcaag

SEQ ID NO: 115 - DNA, 24 bp, Homo Sapiens
acactttcttcactggtcagctcc

SEQ ID NO: 116 - DNA, 23 bp, Homo Sapiens
agacacttgcttcactggtcagc

SEQ ID NO: 117 - DNA, 24 bp, Homo Sapiens
agacactttcttcactggtcagct

SEQ ID NO: 118 - DNA, 23 bp, Homo Sapiens
gacacttgcttcactggtcagct

SEQ ID NO: 119 - DNA, 22 bp, Homo Sapiens
agacactttcttcactggtcag

SEQ ID NO: 120 - DNA, 21 bp, Homo Sapiens
gaggagctgaccagtgaagca

SEQ ID NO: 121 - DNA, 25 bp, Homo Sapiens
ccatgattcctctctgttcccttcc

SEQ ID NO: 122 - DNA, 24 bp, Homo Sapiens
atggttcctctctgttcccttcct

SEQ ID NO: 123 - DNA, 23 bp, Homo Sapiens
gccatgattcctctctgttccct

SEQ ID NO: 124 - DNA, 24 bp, Homo Sapiens
catggttcctctctgttcccttcc

SEQ ID NO: 125 - DNA, 23 bp, Homo Sapiens
ctgccatgattcctctctgttcc

SEQ ID NO: 126 - DNA, 23 bp, Homo Sapiens
ccatggttcctctctgttccctt

SEQ ID NO: 127 - DNA, 26 bp, Homo Sapiens
tttctgccatgattcctctctgttcc

SEQ ID NO: 128 - DNA, 28 bp, Homo Sapiens
ctgccatggttcctctctgttcccttcc

SEQ ID NO: 129 - DNA, 21 bp, Homo Sapiens
tcttgcccacagcaccctcaa

SEQ ID NO: 130 - DNA, 21 bp, Homo Sapiens
tcttgcccacagcaacctcaa

SEQ ID NO: 131 - DNA, 23 bp, Homo Sapiens
tttgagggtgctgtgggcaagat

SEQ ID NO: 132 - DNA, 23 bp, Homo Sapiens
tgaggttgctgtgggcaagatgc

SEQ ID NO: 133 - DNA, 20 bp, Homo Sapiens
cccacagcaccctcaaacct

SEQ ID NO: 134 - DNA, 20 bp, Homo Sapiens
cccacagcaacctcaaacct

SEQ ID NO: 135 - DNA, 27 bp, Homo Sapiens
gcaggtttgagggtgctgtgggcaaga

SEQ ID NO: 136 - DNA, 21 bp, Homo Sapiens
gaggttgctgtgggcaagatg

SEQ ID NO: 137 - DNA, 24 bp, Homo Sapiens
cgacatcgaaagtaaatttgccct

SEQ ID NO: 138 - DNA, 27 bp, Homo Sapiens
atcaacatcgaaagtaaatttgcccta

SEQ ID NO: 139 - DNA, 29 bp, Homo Sapiens
attttatcgacatcgaaagtaaatttgcc

SEQ ID NO: 140 - DNA, 30 bp, Homo Sapiens
attttatcaacatcgaaagtaaatttgccc

SEQ ID NO: 141 - DNA, 29 bp, Homo Sapiens
ccagaaagaagagattttatcgacatcga

SEQ ID NO: 142 - DNA, 30 bp, Homo Sapiens
ccagaaagaagagattttatcaacatcgaa

SEQ ID NO: 143 - DNA, 26 bp, Homo Sapiens
ttatcaacatcgaaagtaaatttgcc

SEQ ID NO: 144 - DNA, 30 bp, Homo Sapiens
agcctgacttcttattcagagacttgtcat

SEQ ID NO: 145 - DNA, 30 bp, Homo Sapiens
cctcacttcttattcagagacttgtcatgg

SEQ ID NO: 146 - DNA, 27 bp, Homo Sapiens
acaagtctctgaataagaagtcaggct

SEQ ID NO: 147 - DNA, 27 bp, Homo Sapiens
acaagtctctgaataagaagtgaggct

SEQ ID NO: 148 - DNA, 29 bp, Homo Sapiens
agcctgacttcttattcagagacttgtca

SEQ ID NO: 149 - DNA, 30 bp, Homo Sapiens
agcctcacttcttattcagagacttgtcat

SEQ ID NO: 150 - DNA, 32 bp, Homo Sapiens
acaagtctctgaataagaagtcaggctggtga

SEQ ID NO: 151 - DNA, 30 bp, Homo Sapiens
acaagtctctgaataagaagtgaggctggt

SEQ ID NO: 152 - DNA, 27 bp, Homo Sapiens
cctcacttcttattcagagacttgtca

SEQ ID NO: 153 - DNA, 29 bp, Homo Sapiens
gctgcttcttttggctctgactttattga

SEQ ID NO: 154 - DNA, 28 bp, Homo Sapiens
gctgcttcttttggctctgactttactg

SEQ ID NO: 155 - DNA, 29 bp, Homo Sapiens
atgagatcaataaagtcagagccaaaaga

SEQ ID NO: 156 - DNA, 29 bp, Homo Sapiens
atgagatcagtaaagtcagagccaaaaga

SEQ ID NO: 157 - DNA, 28 bp, Homo Sapiens
agatcaataaagtcagagccaaaagaag

SEQ ID NO: 158 - DNA, 28 bp, Homo Sapiens
agatcagtaaagtcagagccaaaagaag

SEQ ID NO: 159 - DNA, 30 bp, Homo Sapiens
gctatgagatcagtaaagtcagagccaaaa

SEQ ID NO: 160 - DNA, 26 bp, Homo Sapiens
agatcagtaaagtcagagccaaaaga

SEQ ID NO: 161 - DNA, 29 bp, Homo Sapiens
ctctttgcaattctaattaacctcaatgt

SEQ ID NO: 162 - DNA, 29 bp, Homo Sapiens
ctctttgcaattctatttaacctcaatgt

SEQ ID NO: 163 - DNA, 30 bp, Homo Sapiens
agcctctctttgcaattctaattaacctca

SEQ ID NO: 164 - DNA, 30 bp, Homo Sapiens
agcctctctttgcaattctatttaacctca

SEQ ID NO: 165 - DNA, 30 bp, Homo Sapiens
tgaggttaattagaattgcaaagagaggct

SEQ ID NO: 166 - DNA, 30 bp, Homo Sapiens
tgaggttaaatagaattgcaaagagaggct

SEQ ID NO: 167 - DNA, 32 bp, Homo Sapiens
acattgaggttaattagaattgcaaagagagg

SEQ ID NO: 168 - DNA, 33 bp, Homo Sapiens
ctctctttgcaattctatttaacctcaatgttg

SEQ ID NO: 169 - DNA, 21 bp, Homo Sapiens
aagcccactgggcaacagtgg

SEQ ID NO: 170 - DNA, 20 bp, Homo Sapiens
gcccactgggtaacagtggc

SEQ ID NO: 171 - DNA, 22 bp, Homo Sapiens
gtaagcccactgggcaacagtg

SEQ ID NO: 172 - DNA, 22 bp, Homo Sapiens
aagcccactgggtaacagtggc

SEQ ID NO: 173 - DNA, 21 bp, Homo Sapiens
gggcaacagtggctggggaaa

SEQ ID NO: 174 - DNA, 20 bp, Homo Sapiens
gggtaacagtggctggggaa

SEQ ID NO: 175 - DNA, 25 bp, Homo Sapiens
tgtaagcccactgggcaacagtggc

SEQ ID NO: 176 - DNA, 26 bp, Homo Sapiens
ccactgggcaacagtggctggggaaa

SEQ ID NO: 177 - DNA, 25 bp, Homo Sapiens
gtaagcccactgggtaacagtggct

SEQ ID NO: 178 - DNA, 23 bp, Homo Sapiens
actgggtaacagtggctggggaa

SEQ ID NO: 179 - DNA, 23 bp, Homo Sapiens
tcggcctcccacagttttgtgat

SEQ ID NO: 180 - DNA, 23 bp, Homo Sapiens
cagcctcccacagttttgtgatt

SEQ ID NO: 181 - DNA, 23 bp, Homo Sapiens
ctcaggtgatctgcctgcctcgg

SEQ ID NO: 182 - DNA, 22 bp, Homo Sapiens
tcaggtgatctgcctgcctcag

SEQ ID NO: 183 - DNA, 24 bp, Homo Sapiens
cggcctcccacagttttgtgatta

SEQ ID NO: 184 - DNA, 24 bp, Homo Sapiens
ctcagcctcccacagttttgtgat

SEQ ID NO: 185 - DNA, 27 bp, Homo Sapiens
tgcctcggcctcccacagttttgtgat

SEQ ID NO: 186 - DNA, 28 bp, Homo Sapiens
ctgcctcagcctcccacagttttgtgat

SEQ ID NO: 187 - DNA, 23 bp, Homo Sapiens
tgatacacggctgactcccccac

SEQ ID NO: 188 - DNA, 23 bp, Homo Sapiens
tgatacacggctgactccccacg

SEQ ID NO: 189 - DNA, 22 bp, Homo Sapiens
ggctgactcccccacgtgtcca

SEQ ID NO: 190 - DNA, 22 bp, Homo Sapiens
cggctgactccccacgtgtcca

SEQ ID NO: 191 - DNA, 25 bp, Homo Sapiens
gatgatacacggctgactcccccac

SEQ ID NO: 192 - DNA, 25 bp, Homo Sapiens
gatgatacacggctgactccccacg

SEQ ID NO: 193 - DNA, 21 bp, Homo Sapiens
tgctgcctatacagtcacttt

SEQ ID NO: 194 - DNA, 21 bp, Homo Sapiens
aaagtgactgtataggcagca

SEQ ID NO: 195 - DNA, 20 bp, Homo Sapiens
aaagtgactgtataggcagc

SEQ ID NO: 196 - DNA, 25 bp, Homo Sapiens
ctagacctgctgcctatacagtcac

SEQ ID NO: 197 - DNA, 20 bp, Homo Sapiens
caggtttcacctctcccacc

SEQ ID NO: 198 - DNA, 21 bp, Homo Sapiens
ccaagatcacccaaagtccca

SEQ ID NO: 199 - DNA, 20 bp, Homo Sapiens
ccggtggggaattttcttgg

SEQ ID NO: 200 - DNA, 21 bp, Homo Sapiens
gaggggaaaggagggggttga

SEQ ID NO: 201 - DNA, 20 bp, Homo Sapiens
ccggtggggaattttcttgg

SEQ ID NO: 202 - DNA, 21 bp, Homo Sapiens
gaggggaaaggagggggttga

SEQ ID NO: 203 - DNA, 20 bp, Homo Sapiens
cgccgcttcctttgtccatc

SEQ ID NO: 204 - DNA, 21 bp, Homo Sapiens
tccgtaccttcaccccctcct

SEQ ID NO: 205 - DNA, 22 bp, Homo Sapiens
tggagagtgctggtgtacccca

SEQ ID NO: 206 - DNA, 20 bp, Homo Sapiens
gcctccacccccaccctgtc

SEQ ID NO: 207 - DNA, 20 bp, Homo Sapiens
ggcccactccccacagctct

SEQ ID NO: 208 - DNA, 21 bp, Homo Sapiens
ccccctccatcccacccatca

SEQ ID NO: 209 - DNA, 22 bp, Homo Sapiens
agccagacaaggttgttgacac

SEQ ID NO: 210 - DNA, 22 bp, Homo Sapiens
cagaggactcttggtctttccc

SEQ ID NO: 211 - DNA, 21 bp, Homo Sapiens
cttgaaggaaatgccccatta

SEQ ID NO: 212 - DNA, 22 bp, Homo Sapiens
tgcccagtgcttaacaagacca

SEQ ID NO: 213 - DNA, 21 bp, Homo Sapiens
tcatccctattggcaggttac

SEQ ID NO: 214 - DNA, 22 bp, Homo Sapiens
gcaagtgatgcccatgtcggtg

SEQ ID NO: 215 - DNA, 24 bp, Homo Sapiens
ctttggggagctgaaggactacta

SEQ ID NO: 216 - DNA, 23 bp, Homo Sapiens
cactttgtgaccattccggtttg

SEQ ID NO: 217 - DNA, 22 bp, Homo Sapiens
tttgcatatttttttgaactct

SEQ ID NO: 218 - DNA, 21 bp, Homo Sapiens
actatttgtttttaactgtga

SEQ ID NO: 219 - DNA, 17 bp, Homo Sapiens
gagtggaaatccatgtt

SEQ ID NO: 220 - DNA, 22 bp, Homo Sapiens
taagatgattcattctgacagc

SEQ ID NO: 221 - DNA, 20 bp, Homo Sapiens
ttgtgtaagaaaacaaaggt

SEQ ID NO: 222 - DNA, 22 bp, Homo Sapiens
ctaatttttgtatttttagtag

SEQ ID NO: 223 - DNA, 18 bp, Homo Sapiens
gcaaacagccaggtatag

SEQ ID NO: 224 - DNA, 20 bp, Homo Sapiens
ccccaacaccaaatatacac

SEQ ID NO: 225 - DNA, 20 bp, Homo Sapiens
ttgaaatgccacagacattc

SEQ ID NO: 226 - DNA, 22 bp, Homo Sapiens
ctatgtagactttccttgatat

SEQ ID NO: 227 - DNA, 18 bp, Homo Sapiens
catgggcatttgattggc

SEQ ID NO: 228 - DNA, 19 bp, Homo Sapiens
gtgaaatgggaagctctgt

SEQ ID NO: 229 - DNA, 18 bp, Homo Sapiens
tcgaggtagtgtttacag

SEQ ID NO: 230 - DNA, 18 bp, Homo Sapiens
gctttgtactcacatctc

SEQ ID NO: 231 - DNA, 17 bp, Homo Sapiens
gtccaaggagctgcagg

SEQ ID NO: 232 - DNA, 17 bp, Homo Sapiens
tcgcggatggcgctgag

SEQ ID NO: 233 - DNA, 17 bp, Homo Sapiens
gtccaaggagctgcagg

SEQ ID NO: 234 - DNA, 17 bp, Homo Sapiens
tcgcggatggcgctgag

SEQ ID NO: 235 - DNA, 22 bp, Homo Sapiens
aagaattatttttgtgtttcta

SEQ ID NO: 236 - DNA, 22 bp, Homo Sapiens
gtggattcttaagtcttcttta

SEQ ID NO: 237 - DNA, 19 bp, Homo Sapiens
tccagcccttagctcacct

SEQ ID NO: 238 - DNA, 24 bp, Homo Sapiens
ctcccatcctttctcccatttctc

SEQ ID NO: 239 - DNA, 19 bp, Homo Sapiens
tctgtcagtctcattgtct

SEQ ID NO: 240 - DNA, 19 bp, Homo Sapiens
gagagagatcgacagagaa

SEQ ID NO: 241 - DNA, 17 bp, Homo Sapiens
cctcaactctgttctcc

SEQ ID NO: 242 - DNA, 19 bp, Homo Sapiens
gtggatgtttaccaatgag

SEQ ID NO: 243 - DNA, 18 bp, Homo Sapiens
aatcaagcaaccctccag

SEQ ID NO: 244 - DNA, 18 bp, Homo Sapiens
ggaatgaggtggcaagtg

SEQ ID NO: 245 - DNA, 20 bp, Homo Sapiens
ggtgatcttctgttctaggg

SEQ ID NO: 246 - DNA, 18 bp, Homo Sapiens
atggggtgaagaggaatg

SEQ ID NO: 247 - DNA, 22 bp, Homo Sapiens
tgcttgagttgtagaaagaacc

SEQ ID NO: 248 - DNA, 18 bp, Homo Sapiens
ttacaacagtctccagcc

SEQ ID NO: 249 - DNA, 601 bp, Homo Sapiens
tgtttgtctgtctgtctgctgctcctagtctctgcctctcccagtctctcagcttccgtttctttcttaaactttctctcagtctctgaggtctcgaaatcacgaggcttcgacccctgtggaccagatgcccagctagtggcctttctccagcccctcagatgacacagaactacaaaccccagcatgcactctggcctgaagtgcctggagagtgctggtgtaccccacctgcattctgggaactgtagtttccctagtcccccatgctcccaccagggcatcaagctcttccctggcyggctgaccctgcctcagccctagtctctctgctgacctgcggccccgggaagcgtgcgtcactgaatgacagggtgggggtggaggcactggaaggcagcttcctgctcttttgtgtcccccacttgagtcatgggggtgtgggggttccaggaaattggggctgggaggggaagggataccctaatgtcagactcaaggacaaaaagtcactacatccttgctgggcctctatccccaagaacccaaaaggactcaagggtggggatccaggagttcttgtatgtatggggggaggtga

SEQ ID NO: 250 - DNA, 850 bp, Homo Sapiens
tccccatgcgtgccagctcggccatcacagtgttcccgcagcgctgccctggccgaggagacttccgaatctggaacagccagctggtgcgctacgcgggctaccggcagcaggatggctctgtgcggggggacccagccaacgtggagatcaccgaggtgggcaccgagggccacccatgagggtgtccccaaggtggagaatgaggaaaccagtgggagaaggctcgggggatccaggcaggaagaggggagcctcggtgagataaaggatgaaaaacaccaaaggaggggtgcctgggtggtcacggagacccagccaatgagggaccctggagatgaaggcaggagacagtggatggaggggtccctgaggagggcatgaggctcagccccagaaccccctctggcccactccccacagctctgcattcagcacggctggaccccaggaaacggtcgcttcgacgtgctgcccctgctgctgcaggccccagatgakcccccagaactcttccttctgccccccgagctggtccttgaggtgcccctggagcaccccacgtgagcaccaaagggattgactgggtgggatggagggggccatccctgagcctctcaagaagggcctgcaagggggtgctgatcccacaccccaacacccccaggctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggtgaggtg

SEQ ID NO: 251 - DNA, 601 bp, Homo Sapiens
tgtctgtagtcctgggggtctataattctggcttttgaacttggcgatactctattttagaaattgtatcatttagcctccctatacccttattatattgagcaagagactgaggcccagagagggcaggggagctggccaaagtcacacaacagtcagtgatggagcctccgtcacctgagctcatggcaacccctgactttggccatagagtgacccccaacaccaaatatacaccaacctcctaatctttaccccctgactcaaccccctaacctggctcagatctgaaccctaactygaaccccagtgattctgggtctcagacaaacacaaatccctatacctggctgtttgcaatgcagctaggaccttctcaccaaagaacttgaccttgaaggcgaggtcaggagggggtgatgacagctccactcagcccatctccctcccagggcactcctctctctgggctagtgtggcagccggcttcagggctggcttcaggccagggaggctcctggcatagtgggtgtccatgaaaagacccctggcagggcagacagatgcactgatacttacacaccaggagggcaggcttggt

SEQ ID NO: 252 - DNA, 601 bp, Homo Sapiens
gtggtggtatggattggggtttaggcagaacagtactggccaagcagcgcctccctggacctcaattttccctctgtggaatgggctagcaatcctgggcctccccagggcgaaggaaagaccactcaggaagggcaccgtctggggcaggaaaacggagtgggttggatgtatttttttcacggatgggcatgaggatgaatgcttgtccaggccgtgcagcatctgccttgtgggtcacttctgtgctccagggaggactcaccatgggcatttgattggcagagcagctccgagtccrtccagagcttcctgcagtcaatgatcaccgctgtgggcatccctgaggtcatgtctcgtaagtgtgggctggaggggaaactgggtgccgaggctgacagagcttcccatttcaccttgtgggcccttcccaggcagagcttcaggtgcccctcttcccagtcattgatacttagcggtcctggccccctttcctctccctgctggtggtattgcacgccaatgactcggccagatgcccagacccctgttcttggtttacctgcagaatattatctttgccaccccgcgggatggctca

SEQ ID NO: 253 - DNA, 401 bp, Homo Sapiens
tcactctgctaaatcaaagtgaaacgcatgtttacagaatattggtccaaaagggtctcagcatctcccactacccagggtggcagagcctcgggccggccttgctccccaagaagggctgactggggctctgtcccctgccccagggctcgaggtagtgtttacagccctcatgaacagcaaaggcgtgagcctcttcgrcatcatcaaccctgagattatcactcgagatgtgagtacaaagcccccctcaccagcccctgttcctggggagagaggcccagacaggattcctggggtgactgggggctgttggggagacagacagaggggcctctaccagcttggctccctcctggtggcctgggagtcagcccagctcgcccctctctcctactgcc

SEQ ID NO: 254 - DNA, 801 bp, Homo Sapiens
cctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgygcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggagga

SEQ ID NO: 255 - DNA, 511 bp, Homo Sapiens
gcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagygcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagc

SEQ ID NO: 256 - DNA, 686 bp, Homo Sapiens
ggcctgtcggggggggggggtggggggcggggggagggatagcattaggagatatacctaatgttaatgacaagttaatgggtgcagcacaccaacatgacacatgtatacatatgtaacaaacctgcacgttgtgcacatgtaccctagaacttaaagtataatttaaaaaaaataaaaataaaagaattccttttgcaatattaattggttccagcgaaagcttatttatttatttattatcatgaaataactttgcaaatgaaaacaattttgaatatattttctttcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcraggaatacaggtattttgtccttgaagtaacctttcagaaattctgagaatttcttctggctagaacatgttaggtctcctggctaaataatggggcatttccttcaagagaacagtaattgtcaagtagtcctttttagcaccagtgtgataacatttattctttttttttttttgtcttgtctatt

SEQ ID NO: 257 - DNA, 601 bp, Homo Sapiens
grggaatactgatgtgaccttgaacttgactctattggaaacctcatctttcttcttcagagcccctttaacaaccgctggtatcaaatgggcatcgtctcatggggtgaaggctgtgaccgggatgggaaatatggcttctacacacatgtgttccgyctgaagaagtggatacagaaggtcattgatcagtttggagagtagggggccactcatattctgggctcctggaaccaatcccgtgaaagaattatttttgtgtttctaaaactatggttcccaataaaagtgactctcagyragcctcaatgctcccagtgctattcatgggcagctctctgggctcaggaagagccagtaatactactggataaagaagacttaagaatccrccacctggtgcacgctggtagtccgagcactcgggaggctgaggtgggaggatcgcttgagcccaggaggtggaggctgcagtgagccactgcaccccagcckrggtgacagagygagaccctgtcccaaaagaatccactatctatcttcagagcagggccaggtgagaggaaagatggcaggttgaatttacaggcattaaagatgt

SEQ ID NO: 258 - DNA, 801 bp, Homo Sapiens
ctcagtttcctgtctatatctagtgccaccaggcatcagaaacgtaagtgcttcaaggatcttgggtgaaaagccgctttagcggcgagcatacactaattaataaaatgccttggccggggctgggcagaggaagtaagcgggcagccgaggtgacagctggagggaggagcgggggtggagccgggggaagggtggggaggggatgggctggagctccgggcagtgtgcgaggcgcacgcacaggagcctgcactctgcgtcccgcaccccagcagccgcgccatgagccgtgagtgcgaccccggtgcccggtggggaattttcttggcctcctggtggagccttgaatgccaggctcagcccctcatctctctcctctgcagggagtctcttgctcyggttcttgctgttcctgctcctgctcccgccgctccccgtcctgctcgcggacccaggggcgcccacgccaggtaggcggccccatccctccccaagggaatccccggtcttgcgcccctggcctggtttcaaccccctcctttcccctccagcgggcccagcttcccctttctgctcgcggtgctgagaaagactgaggctgagtcttttggtgggatgggggctccctgaagcccccccggcggtgtggccttggctaatgggctatctagttctttcaggggaaacagcagactgggatctggtgccaacttggggagaagggacagtccctatccatccccctcacctgttctgggccccagatgtctaagcagcctctgcacccaacaaccccgc

SEQ ID NO: 259 - DNA, 511 bp, Homo Sapiens
gggggtggagccgggggaagggtggggaggggatgggctggagctccgggcagtgtgcgaggcgcacgcacaggagcctgcactctgcgtcccgcaccccagcagccgcgccatgagccgtgagtgcgaccccggtgcccggtggggaattttcttggcctcctggtggagccttgaatgccaggctcagcccctcatctctctcctctgcagggagtctcttgctccggttcttgctgttcctgctcctgctccygccgctccccgtcctgctcgcggacccaggggcgcccacgccaggtaggcggccccatccctccccaagggaatccccggtcttgcgcccctggcctggtttcaaccccctcctttcccctccagcgggcccagcttcccctttctgctcgcggtgctgagaaagactgaggctgagtcttttggtgggatgggggctccctgaagcccccccggcggtgtggccttggctaatgggctatctagttctttcagg

SEQ ID NO: 260 - DNA, 511 bp, Homo Sapiens
aactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccscctctctttccaagaaacaaggagggggtgaaggtacggagaacagtatttcttctgttgaaagcaacttagctacaaagataaattacagctatgtacactgaaggtagtagctatttcattccacaaaataagagttttttaaaaagctatgtatgtatgtgctgcatatagagcagatatacagcctattaagcgtcgtcactaaaacataaaacatgtcagcctttcttaaccttactcgccccagtctgt

SEQ ID NO: 261 - DNA, 801 bp, Homo Sapiens
tcttttctatggccaccaagtgcaggcctgatttgcttggctgctcaaggcaggacagtgtgggagtttggagcaatccacccccactcttggaactgggctctgagccacctcccctgagagtcatctctggggtcagaagcatatcagtcatgagcccagccactcactgttttagttcaggctgtgctgtgctgttggaaggtgcaagatcagagcccccaaagcagaggactctctctgcccagtccctgtggtctcttcatccctcgccttgaacaggtggaggccagcctctcctgactgtcatccctattggcaggttaccccaaaggccaccccgaagcagggagctttgaggctgacctgaagcacttgaaggagaaggtgtctgcgggagycgatttcatcatcacgcagcttttctttgaggctgacacattcttccgctttgtgaaggcatgcaccgacatgggcatcacttgccccatcgtccccgggatctttcccatccaggtgaggggcccaggagagcccataagctccctccaccccactctcaccgcaccgtcctcgcacaggctgggggctctgggtggagtgctgagttcgctgagttcttcccagatctcctctcaggtccagaacttgcacagcgttgcttggccaccccattttggttacctctaattttccccccaaaacccagcaacagtgtctgttgaggggtttgttgtactttggccaacaagcatcaccaaaagggattctaattctcattacaaatcctgcttaaatcag

SEQ ID NO: 262 - DNA, 801 bp, Homo Sapiens
aggaagatgtacgtcccatcttctgggcctccagaccaaagagttacatctaccgtacccaggagtgggacgagttccctaacggccgctggtgagggcctgcagaccttccttgcaaatacatctttgttcttgggagcgggagggcagaagaagtttgcatgcttgtggttgacctgggaggagtcaggggcagaatttacaggaatggcctcctgggcatgtggtggcactgccctctgtcaggagtgtgccctgacctctgggcacccctctgccaggggcaattcctcttcccctgcctttggggagctgaaggactactacctcttctacctgaagagcaagtcccccaaggaggagctgctgaagatgtggggggaggagctgaccagtgaagmaagtgtctttgaagtcttcgttctttacctctcgggagaaccaaaccggaatggtcacaaagtgagtgatgctggagtggggaccctggttcatcccctgcccctggcctgaccccagctgcaggccaggctgcggggctgtgacttccccatcctgtgccctcccctccatgctgtggacatggcaaagggagaagggtaagttgggagacctccacctggaagggcttagggaggcaaagacaggctgggtctttgttgggggccgtgagagggactcagggtgccaaacctgatggtcgccccagccagctcaccgtctctcccaggtgacttgcctgccctggaacgatgagcccctggcggctgagaccagcctgctgaaggaggagctgctgcg

SEQ ID NO: 263 - DNA, 801 bp, Homo Sapiens
agggagcccctaggggagaacagagttgaggggggctctagggctcaaggtttggctgagccaccccagcagcccccattctcctgctgcctcacctgggccccaggcagcagaaccagcagcagccccagaaggaggaggtgtagggtggtgccacacacccttgggaggaagagacgttcaggtggtgtcatggggagaacctgcagagaaagagagagagagagagagacagtgagcggggcggggcacgcggcggaagacagacctcccgccctgggagacagcaccccccgacccccgagagagagatcgacagagaaggggacaagatgcagtcagagaaaccccaaggtgagcagagggagacagagagagacaggaagggaacagagaggaaycatggcagaaacagagaatgtgtgacagagacaatgagactgacagatggagagtcagagacagagaaggaaaccaaaaccaaacccaccaaggcccaggcccaggcaggccggggatccaggcagcaggtgcaggagggaccgaggcccaggcagagggcaggacactgcggggcggtagtccaaagcacgaagcacgggcagcccaaggagatggggcaggagagcctcacctgctgtgtggagcccctgggcccggacgctcaggtccctttatagaggaagcggcagtggcagcgtggcaggcagcgggcgggttctaggtcggggctggggcccggggaagcccccagggcttagaagatactgctgtttcagtcaaaggcaggaaaggctgagg

SEQ ID NO: 264 - DNA, 801 bp, Homo Sapiens
tgtccccttctctgtcgatctctctctcgggggtcggggggtgctctctcccagggcgggaggtctgtcttccgccgcgtgccccgccccgctcactgtctctctctctctctctctttctctgcaggttctccccatgacaccacctgaacgtctcttcctcccaagggtgtgtggcaccaccctacacctcctccttctggggctgctgctggttctgctgcctggggcccaggtgaggcagcaggagaatgggggctgctggggtggctcagccaaaccttgagccctagagcccccctcaactctgttctcccctaggggctccctggtgttggcctcacaccttcagctgcccagactgcccgtcagcaccccaagatgcatcttgcccacagcamcctcaaacctgctgctcacctcattggtaaacatccacctgacctcccagacatgtccccaccagctctcctcctacccctgcctcaggaacccaagcatccacccctctcccccaacttcccccacgctaaaaaaaacagagggagcccactcctatgcctccccctgccatcccccaggaactcagttgttcagtgcccacttcctcagggattgagacctctgatccagacccctgatctcccacccccatcccctatggctcttcctaggagaccccagcaagcagaactcactgctctggagagcaaacacggaccgtgccttcctccaggatggtttctccttgagcaacaattctctcctggtccccaccagtggcatctacttcgtctactc

SEQ ID NO: 265 - DNA, 6321 bp, Homo Sapiens
tcctgaaagtcagaaagatgaatggattaaaagtatgaaaataggaaaataaatgccttaaaaccaagctctcttacatgtatagaatgctaatatttaatgaaaagaaaacacttcagtggtttttactttcaatacgttaatagaatgcttgatttagtcattcattaaaaaatatttaccaagttactaccatgtgccggggagtgcatgaggcactggggataaagcaatgaacatgacagggaaggtccctatctttaaagtatttgtgttttaggaaagagagatagatagtaaacaagtaggtaacaaatacacatgctgctttcaggtgatgaaaactattatggaggaagtagaccctgactgccagagaggaatggtgccgtcctacctactcggtctgggtcatcaggaagacctcttgcaggagtgatgggtttgttgagccctgaagttgaagaccagccaagggaatgtcctggggacgagggaaaagcaagtgaaagcctttggcgtgggaaagggcttggagggatgagaagggaggccattgggactgaggcagcagaggagagagggaggagagaatgtgtagaataagatgagagggtgggtggttggatgtggcccagagtatgcaggaccttgaggctgcaataagaggttagaacatgattcaaattgcaatgcgatgatgtgaaaccattgtaggctttcttttttctgtttttaaaaattgaggtgtaactcacacttagcaaactgcatatatttaaagaatgtaattgtataagttttgacatatgtatacctacatgaaaccatttccacaatcaagttcgtggacatattcatcaccctcaaagctcttctcatgctactttataaccccttccttgtagcctccttttcttgactccccacccatcctaggctgccactgatctgcttcctgccactataaatttgtttaatattttaaaaacttttaaaattggtaatagtttacatatttttgatgtacacatgctatcttgatacctctatatacaatgtgtagtcaccaaatcagggtaattgggatattcatcacctcaaacattttcttttctttgtgttgggaacattacaattcttctagctgttttgaaatacattgggaggctgagatgggtggatctcaatgtcaggagtttgagactagcctggccaatatggtgaaaccctgtctctactaaaaatacaaaaattagccaggcgtggtagtgtgtgcctgtaatcccagctactcaggaggctgaggcaggagaatcgcttgaacccaggaggtggaggtttcagtgagtcaagatcgtgccactacactccagcctgggtgacagagcgagactccatctcaaaaaaaaaaaaaaaaaagaaagaaatatgtaaatatacaataaattattgttaactataatttttctactatattatttaatactagaacttgtttcttctaactgtagttttgtacctcttaaccaacctgtcttcaatctaccctcctgtcttcccatccctggcttggtaaccactattttactctcaacctctgtgagatctgcccttttagtgcccacgtatgagtgagaacaggcaatgtttgtctttctgtgccttcttaattcacttactctaatgacctccagttacatctatgttgctgcaaatgacaggacttcattcctttttatggttgagtaatattctgttgtggataattaccacattttctttattccctcatcattgatgggcacttacattgattccatattgtggctatggtgaatagtgctgcaataaacatgaaagtgcagatatactgattgccctttttttttttgggatagatacccagcagtgggatgctggatcatatggtacttctatttttagttttttgagaaatatctatactgctttccaaaatggctgtactactttatgtttctacaaacagtgtatgagcattcccctttctctacattcttgacaacatttgttatttttcatcttcttgataatacccattctaactgaggtgagatagtatatcattgtagttttgatttgcatttctctgatggtcaatttctctgatggtcaatgattttgaacactttttaataaacctttttgccatttgtatgcctacttttgagaaacatctattcaggtcttctgctcatttttaaatcagatttttttttttttttgctgttgaattgtttgagtttcttacatgttctggttattaatccattgtcagatgggtagtttgcaaatattttctccttttttgtaggttgcctcttcacgttgttgtttgttttctttgctgtgcagaaactttttaggttgatgtaatcccatttgtctaattttgctttggtcacctgtgcttttgaggtctttttcaaatctttgcccagaccaatatcctgtaatgtttccccaatgttttcttttagtaatttcagttttcagtggtttcagatcttccatttaaatgcttaatccatgttgagttgatttttgtatatggtgaaagatgggggtctagtttcattttctgcgtattgattcttaccgttttcccaacactatttattaagagactgtcctttcctcagtgtatgttcttggcaactttgtcaaaaatgagttgactattaagtgcatggatttatttctgggttctctgttctgttccactgatctgtgtctacttttatgctagtaccatgctgttttggttactatagcttggtaggatattttgaaattaggtagtgtgatgcctccagctttgttctttttgctcaggattgtgttagctaattggggtcttttgtggttccatacaaaatttaggattatttttctatttctgtgaagaatgttatttgtattttgataggaattgcattgaatcttatagatgttttgggaagtatagatattttaacaatattaattcttccaatctatgtgtatgggatatctttgctttttgtgtgtgctctcttcaattttttttatcagtgtcttatgtcttccttatagagatttttcactccttcggttaaatttattcctaggtatttaatattttgtagctcttatacgtgggattgtcttcttgatttctttttcagattgatcactgttagtatgtataaacactatggatttttgtgtgttgattttatatactgcaactttactgaatttgtttctcagttctgagagatttttgtggagtctttaggtttttctaaatataagatcatgtcatcggcaaacaagaataatttgacttcttcctttccaatttaatttttttactttttaaagaactttacataaagacaattgtatggtaagtattctttaaagtctaacttatgttccctagcataattattttgagtctcatctgtgtgtgtttctttattgccaagtagtatttaattgtgtggatatactaaaatttgtttattcattgaccctgttaatggacatttgtttttggtcttgttttttccagtttctgtctattgcaaataaagctgctaagaacgtttgtatacagatctttgtatgaactattttattttttcttggataaattcctaggagtgtaatcgctggatcatatggtagctctgtgtataacatttaaaaaagccatccaacctattttccaaagtggttgtagccttttacattcccactaccaatgtgtgagaattttagttttttcacatccttgttaacagttggtattgctagttttaaattccagcctttctaatcaacatttagtagtttaatttgtattttcctaatgactatgatgttgaatatcttttaatttgcttattagctatacatatattaatattttcttttgtgcagtgtatgttcaaatcattttgcaatgtttaaaattgggctttttgttttcttaatattgaattttgagtgttctttgtatattccagatacaaggtctttatcggatttgtgctttgtgaatattttccctcagtccgagtcttttcattcttttaacagtgtgtctttataaatttattcttttttttcttttatggatcatgattgtggtatcatatctaggaaatctttgcctgatccaagggctacacagatttttcctcatgttggttttctaaaagttttatggtttagattttacattaggttttcatctgatctatgtttgagtatatttttctatatgaaatgaggtataaacaaaaggttttttttgcttatgaatatctaattgtttcagcaccattttttaaaaagactgtcttttctccatggaattgccatttattctttgtcaaaaatcagttatctatataatgtgagtctatttcagtattctctatttcattccattaatctatttgatatttgtctaatcaataataatcacactgtcttgatgactatacttttataatgaaagcaggtagtgatagtgttacaactttcttctttattgaaattgttttaattatgytagatcctttccattttcctgtgaattttagaaagcctgttaatttctcaaaaaatacatgctggaattttggttgggattgtgttgaatcaataggctaaatggggagtgttgctctcttaacaatagtgagtcttctaaatgctgaacaagatatatttagcttttcatttctttaggtcttctttaattcttctctgcaatattttatagtttttagtgtatacgttcttcatatctgctgttggacttttctctaagtatttaatgtttttaaagctattgcaaactgcattttagaaaactttctgattattcattgctagtatatagaaataaaattgattctttggggtattggtaatgtattcttcaaccttgctaaactcactagttctagcagcatttttgtagagtatattgaattttctcaaggatgattatgttatctgcaaataaagacacttttatttcatagtttgtaagctggaaaacacatattttattttcttgcttgattatacttactggaagctccagtacgatgttgaataggtggtaggaatagacatcattgtcttattcctgattttaggaaggaaagtattcactcttttatgattaagtatgattttatctgtaggtttttcatagatgtcctctatcagattgaggagttttcattcttttcctagtttgcagagagttatttttatcagaaatcgatgttggattgtgtcaattttgttttacatctgatatggtttggctctgtgtccccacccaaatctcaccttgaattgtaataatccccacatgtcaagggcaggaccaggtggagataattgaatcatgaggttggtttcccgcatgctgttctcatgatagtgagtgagtactcacgagatctgatggttttatcaggggcttcccacttcacttggcactccttccccttcctgccaccacgagaagaaggatatatttgcttccccttccaccatgattgtaagtttcctgaggcctctccagccctatagaactgtgagtcaattaaacctctttcctatataaattacccagtcttgggtatgtctttattagaggcatgagaagggactaatacaatatcttttgagacagtaaatacattttagtttgttcgcatagtgaattacagactgattttttgaatgctgaaacaaccttgcatcctggagattaaccctacttggtcatgatgcaatgacctttttttatattgttcaattgattttgctaaaattttgcatctatgtttatgagaccatttacatttaaagtgattataatatgcttaggtttaagcctatcatcttgccatgtgttttctatttgttccatctgttcttaattttcttttcccttttttctgccttcttttggattgagttttttttatgattccatttttatctcctttttggcttcttagctacagttctgttttgttattttaattgttgctttagaatttatagtatacagagcagtttgctgttctccttgtggaacatagaaaaggagtgaagaaaggtagttttggtctcctgcctaggccccacaatctttcctagtggacccaaagctcagggataccttgggttgcaatacttcactgggtatggtaggctcagtgaagttgttaatcctcagcggggcagtttattctg

SEQ ID NO: 266 - DNA, 801 bp, Homo Sapiens
gagatggcagagttgatcttttatcatctcttggtgaaagcccagtaacataagactgctctaggctgtctgcatgcctgtctatctaaattaactagcttggttgctgaacaccaggttaggctctcaaattaccctctgattctgatgtggcctgagtgtgacagttaattattgggaatatcaaaacaattacccagcatgatcatgtattatttaaacagtcctgacagaactgtacctttgtgaacagtgcttttgattgttctacatggcatattcacatccattttcttccacagggtgatcttctgttctagggagaaagtgtctcatttgcagaaaggaaaggcacctgcggtatttgtgaaatgccatgacaagtctctgaataagaagtsaggctggtgagcattctgggctaaagctgactgggcatcctgagcttgcaccctaagggaggcagcttcatgcattcctcttcaccccatcaccagcagcttgccctgactcatgtgatcaaagcattcaatcagtctttcttagtccttctgcatatgtatcaaatgggtctgttgctttatgcaatacttcctctttttttctttctcctcttgtttctcccagcccggaccttcaacccaggcacacattttaggttttattttactccttgaactacccctgaatcttcacttctccttttttctctactgcgtctctgctgactttgcagatgccatctgcagagcatgtaacacaagtttagtagttgccgttctggctgtgggtgcagctctt

SEQ ID NO: 267 - DNA, 601 bp, Homo Sapiens
caggactatatccttgggtgattctactctaacaccacatctcacctattttagacatgccaaatgaaacactctttgtgaatttctgccgagatacaatcttggtgtctcttttttacccagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcartaaagtcagagccaaaagaagcagcaaaatgtacctgaagactcgttctcagatgccctacaaaggtaggctggagactgttgtaaataaggaaaccaaggagtcctatttcatcatgctcactgcatcacatgtactgattctgtccattggaacagagatgatgactggtgttactaaaccctgagccctggtgtttctgttgatagggggttgcattgatccatttgtctgaggcttctaattcccattgtcagcaaggtcccagtgctcagtgtgggatttgcagccttgctcgct

SEQ ID NO: 268 - DNA, 1975 bp, Homo Sapiens
tattctgggtacaggcggatagagcaggaaacaaaacagctacagtgatggacaggtcagcctgcagcaatgcctgcagctctgcaaaggtagctgtatgggtgggcaggtggctagcacttattcagctctggaaggatctcccctctggcctctccctgacacccatcaataaaactgaggagcatcggtggacaggggaccttgtgccccctccctgcctgtgcagttggggctaacccagctacgaagtttgagctcactctctccagctccctctcaattcagagctgaactgtgggaagcttcagagctcctgtttcaaggacaggttctcctcacctctcctaatggaggtgcaccagggaactggccctgctctgcccagggctttccctggactttgccatcatggtctagcaaaccctgttcagattgaggtgagtggtgagatttcgaattctttttgacagaaggattaagtcttcttctgtgggacaagtgggaggtagaggtaagattaaagatggccaaatgtctgagtcctgacagcacaatatggagatctagactttttacagaccacagggcacaggggcctcactaacagagttcccggaagtgatgagtgtctgggggcttcctggttgaagagacactagaatggaccagctgggagctaattttttgggctggagtgtgatggcctgccatcactgcctctgtccctccattgtcacagctgccccttaggagccagctgaggcaatttgtggtcagagtgactttgacagttgtcctgcctgtgttcaggaagggagtttctgtggtccctttgaaaccacagaagagcccctcgtatagctctcatggagggggcaaaacattcaaataactcaggagataacacaactatttgtttttaactgtgagtttttaggcaatcacaagatccagatgtatgtccaagcctctctttgcaattctawttaacctcaatgttgcaaccatagacctaccttaagagttcaaaaaaatatgcaaaaaccctgcctttcttcttcctcataccccaaaatgccattctgaacatttcctgttattaaaaaaagatttccatggtgttaccaggcactgtacacagtctgtgtcccaagacaaggaggtacagttccacatgccccatgactgggttgggctctgcactctctctatactttgagagcctgattttctgtgattgggcagagctggcccaccggtgcaatgtcctcctctgcctttcaaacatgttttagtcatcaagatcttcaaatttgtaaccctttccagcttgatcagcagaatgcagatttggaaaaacagaacgagtttaaaatacatgattctaagaaacctggaccagaactatcaaaactggtttcccagagaatatagcaaatgggctcattggccaatactatgacattggcttttgagaaaagaaaggctttattgaaggctggccagcaaggagacaggagttgggctcaaatctgtctccccagtttggggcttagggcaagttttaattacaagacgcatttcttatgagtagcaggcagagagcctccaacttcttctgcctaggtaccagcagcttagacatgatgcaacctgggaagcacatactgtatttggagaaagtgattgggaagaaatgtgagctgaggggaggggctcagtgcccctgagtacacttagtgatggcagaggaaggatgtcctcccgcaggaggctgttccacatctgctctggttgtagggggagctggaggcattagcagcggcctctttcccccaagagaggcagcctcctccaagttttggcgacattatggccctgcaatcatagggtttgtgagcatagtgctaaggagggaaatggagctgctgttactagttccaccccaacacacacacacacactcacagaa

SEQ ID NO: 269 - DNA, 1974 bp, Homo Sapiens
aacaatgaatgtaggctctgtatacaagttcaggctgctgggcaacttaggccttaagacacaactctgccacttaggcttaagacacaactgacatgatggtgcttaaagtggctgtgatggaaaaggaggctgtttggagcctttggagtgccttttaggtgaaccccagcatagcacctaatgatttggagcaaagctgtgtcattccccaaagataactattcgccttttgagaacatcttctagctactatcaataataaacacagaatgcatcaccatgggccaccgtgttgtcttttgacctgagtttcattgtgaacaagagtcatttgatccaaggcagaaagttgggtgcacacagcagtgttccatcatcaaatggaatatgagttgggcccaagtaggtcctgcagacacaaataagttgcaagagcaagtagtacaggcgcttggcctggccagtactgttccaagttgactgcttcccctcagtctgcatctgtggcttcatggggagtttcctatgaccacttgatggaggaaaaaacaattggagcatagtttatagtgctggtactacccaaagtggctagctgaggcactacatctccactctggggtgcccgtaaggacagtgccaaaggaaaaccccctcagtgagcagaacttggagcaatacaagtgggtgttcattttacctagaagaaagatgtccgtgagttacagatctacacaaaatcacagagagtggttaatcgtttagtctgatggtcagggacttccaaagacatgattagaaaactggtgacaaggagtcctggggaagaggcatatggatacctctgaacacacacaaaacatgagatatgtatcccatatgaatgttaaccaaagagcagccacaacagaagaggattttaaaatcagctgaataagatgattcttctgacagcatcagctagtctctttccccagccactgttrcccagtgggcttacatatatcatggccatgggggagggctatgtatggacacagcaacatgaatttccactcatcaaggccaatttggctccagccattgctgagtgctcagctgccaagatagaaatctacgccaatatggcaccattccctgggctagaaaaccaactggtggaaggttgattacattggccatttccatcatggaaggggcagtgctttgtcttccctggaatagacatttactctggatatggatgtgccttccctgctactacaatgctctgccaaacctaccatccatgggcttaattttatttgttataaaatttcaaccaccattgcttctgccaaggaagtaatcttacagcaaaggaagtacagatatgagcttctgatcatgggcttcactggcctcacagtgaagcagtggccagattagaacagtggaatggattttaaaggctcagttacagcaccagctgggtagcaacaccctgctggcctgggttatgtcctgcaggatgctttaagtcagtgaccaatatatgatgctatttctcccattgtcaggattcatgggtccagaatcatggggtcaaaatgggagtggcttttctcactatcaccctggtgttcgggtagtaatttttccttcccattccttaactttgggctctgctattgcagaaatcttagctcctgtggggggaatgcttccatcagggaatacaatggtggttccctaaactgacagctgagtttgccatctcctcgtgccagtgaatacacaagcaaggaagggggttcctttctcacctaggtgactgatcctaattaccaaggagaaattggactgccacttcacaatgagggtgaggagtatgtactctatgtgtctgtattaatgtcaatagaaagtgacaccaacctagtacacagaggactgatcatggtccaggcccttcaggaatgaagatttagt

SEQ ID NO: 270 - DNA, 1975 bp, Homo Sapiens
tgcctgcctcagcctcccaaattgctgggattacaaggcgtgttgttttaagccactcagtttgtggccacttgttacacagcaagaggaaactcatacagttatcatgtgaactcacaggaatatggtgagttaaaaagagaggaagggtgcaaaactccacggtagagtgagaactctccagggagtgaggactgtgcccagcatacagtgatcaccctcttagtaagctaagttctgagcaccagcttttttgagttgactttgttgtctttaacatttgaagatcacccttctttgctcagcctggcttgcaacctgggctgatttgtggatctgatagaaaagtttccttagttgggctcttctccccgaccacccccatgccagtgtggcacatcctctgtctgcattgctcactcttcaattccaagaagcgcaggggcaccgccaggaacaggaaccctgccagagaatacatcaagaaaccaagtctcccttacgcatcaccgtaggaacagagttaatggattatgaacatgtgtttgctttaaccattgtttgtttcccaggtggcagctggctgccccatcttattgggtagatgtaagtggaattacgaatgggatttagtttcatgcacgatggtgattattaacttcaactttcaggtaattttcagaccacattgcactaacttggtctctgatttttttctccttgtttgtttattctgcagccagaactgtgtagatgcgtaccccactttcctcgctgtgctctggtctgcgggctactttgcagccaaggtaactcagacttccctttgttcattctccttctataaagtgcatctcaaggaggttcaagggcaggctttttgttgaaaggactttgcctgacctctggctcccatctgtgaagccctggagaggtgagagccctcggaggccgtgtttcaggcatgctctgcacccgtgcagagcgcrtgtgataatgcattgctaatgcttgctccctggtgctggctgagagctgctgtgctgacaagggtggtttaaggctaaatgtgactcagaatccttaagcagtgttagttcagtacaagggcattataaatgagagtgcctgagggatctattttgggaccgctgtcacttggctcttctgctaataagcttcagtgtggtggccctccttcaggcatgtttccactgagccacgggctggatgccacatccccggccttcccacagttatagcagcccacaggcttgacttgagcaagttggaaagacaaatcaacttccagagttgatttaacattgagtggaaatcatcatacttttggtcccctttcggggccacgcctggcactgtgcctggtggcagatcggcatgaactggccagcttctgtgccctggagggcacaggcagaaaggccacactcagtcccatgatgaactgtttaagacttattgttgtctccccgctcttaaagtagatagagtggattttatgtcccttattacctttcaggatactttgactcagggagataaagtaacttgggtaagctactcagctggtgaagaacacaggcagaatgagtgcctgggtcttttgacttaaaattctggatttttcacaaagacctcttactttattcatttacataataaatatatattgaagagctactctgtgccaagccctgtgcctagatatacagtataaataaagagtagcttctagaggtcacctggcggtgaggcacaggccagctggcaagatggaccacagaagtcagtgatgaagacaatgacaagggtgggaagcgccatatgggaagagaaccaagttcagtgatagagagcagaggtgaggcggcgcagaaaccacttaagggacaccacgtggcactccttctgtgctgagaaggctgtcagtaagctcaccatttatttccttttt

SEQ ID NO: 271 - DNA, 1975 bp, Homo Sapiens
gcattcttatttatcctctaaaaccacaattccattatctctcctattcttatcaacactgccctaaatgatattctttttctcttttgccctggaaaacctctatcatgccttttcccatgtgattacctcgttaagagtgggggtggaatgtctagaatgaaataagagggtcttctcttttgcctggctccctatgcagccctatcttaccccctgcaaagtcccagggatgtgctcagtcactgctcctctcttcatctgtcaccacttgcttgagatcctacagctgctttaattccgagaccatctgcagacatgacaaaatttgtccacctacccacatgtccttttaactttaaaggctttactaactgattcctattagggaatgacagaggtggcaaaaataaacaataggagattgatttacaagaaatctttaaaatagtagatttcttcggacctcattgaatataaatggcctgccttcttgtgtccctccctggtctccctctttaggtgataagaagaagatcctgccagccccatacccgccatctgcgcgggttctagacccccttctcctcccctctggccgtggtaggcattactgatgaatcatggtgctcttcttccagagaccaaacctggcctcggaatccttcttaacacagatactgcttaacacaaccactctgagcagctgtctaagtagaagtaatagatactagaagaaatgtctaagcctaatctagaccaaaatacggcctgatatagatgcaagccaaggggctttatggttaaatgcaaggagattttcaaccctgccgtctagaagctacttgctgagatcttcttcagttgggccatctcctccccaggcctctcttctgttcctgggctatgtcacacttggactctgcagacacctaatgctcttgggactgctttagttcttgacctcaccaaccgaggaggaattgctmgatgagatccttcccccggaatttctctcttgaacccagatggtccgttgcccctttccagaagttgctccagccctgtccgcttaggaagttcagtgtcatccttgatccaggggtagggaagacattccataatgaatgccccagtctgagcttcttccttcaggcttcaggctgccctgcgaggattttcagctccctttttaatgccctctagaagtttctggctcttattttcagcccttcatcctactctctctgaccccttccttatcctgtttagttcacctgtagcagttactacccagcagtgaaggatgaatcttggtttcgtttcttttctcttctttcttttttctcttctcttttccccttcccttcccttccctcccttcacatcacctcatctcacctcaccttacatagtctgctctgtcacccaaactggagtgcagtggcctgatcttggctcactgcaacctccacctcttcccaggttcaagtgattttatacctcagcctcttgagtagctgagactacaggtgtgcactaccacacccagctaattttttgtatttttagtagaatagggtttagctatgttggccaggctggtctcgaactgctgaactcaagcaatctgccatccccggcctcccaaagtatgggagtataggcataagccacccatgatgcccagcctgaatcttggtttcttccccattcatttaagctattacctggcctgaactcaatggcacctggcaccaactggcaactgactcttggtcttttattacctaccttccctagcaggcactggttgctccctcttcctatcccatggagtcctgtcctctgttggggctcctactgatcctcttggcaatatgaagttctcactcaatggtgggtgggcaatgactgccaactcttgaggccaatgaactcaggttaccccactcctcctcctcctgagttctca

SEQ ID NO: 272 - DNA, 1975 bp, Homo Sapiens
acagagtgagaaagctggtctctaaaaaacaaacaaacaaaaaagaaactgtcaaactcttcccaacatgttgccattttacatttaccattttacattcttaccagcaatgattgatagttccagttgctccatacccttgctgaccattccaatagtgtattgtgttatctcattgtagttctaatttgtatttccctagtgattaatgatgtttaacatcttttcatgcacctatggctatatgtatatcttctttagcaaaatatatgttgttatttgaagagcggaagttttacattttgatgaagtctaattattgattttttttttcttagatggctcatgctttttgtgttatctaaaaaaaatttgccttcttcatggtcacaaagctttctcctatgttttcttttggaagctttatatttttagtttttatgtttatgtttaagacccatttctagttacaattgtgtgattttttggaagggtcaaggttcattttcttttccataagaatgtacagttgttctagcacccttgttaaaaaactttcctttccccattgaactactttgtcaaaaatcaactgagcatatatgggcatcatgaattttaatcctgttagactgaatgttcccaaggcaggccatgcccatgactgacctcctttccttggattgcctacaaaacagataaagctaagtcggagcaaagaaatccatgtctaacctgtatttttttttttttttttttagatggggtctcgctctgtcacccaggctgggtgcagtggcgtgatcccagctcactgcaatctctgcctcctgggttcaagtgattctcctgcctcagcctcccgagggctgggattgtaggcgtgcaccactatgcccatctaatttttgtatttttagtagagatagggttttgccattttggccaactgtcttgaactcctgacctcaggtgatctgcctgcctcrgcctcccacagttttgtgattataggcatgagccccgtgcccggccttaacctttgttttcttacacaacacactacgtgatgttttccacatgcatgggtcatttgcttcattacgtacaaatgcataagcaatatactgtgtggtgtgagtttgtgatgggaaaaggaagaagttttgcggatactacacggcttcctgctatctgtctgtgtgaatggctatggactttgtcttctatttgttcgcttagcgcagatatgatcagcttcaacttaagattctagagaaagagggtcatatctgtaaagcactctgagcatgtgtgaagtttaatcaatagcatatgagttacagcaaattcactatctttgtttcttcagctatagaatggcatgaggattcatctcaatttagttcaattctgttagaaccatgagctagctgttcatggaaggaaagcccacctgattgtggccagggaaggagaaacaacactttaaccaggtgatttggttctcacagacaccattggcatgtgacatctggaacagaccatgcctggtctctgttcgtatcacttactatcagctcaatattggtctgaatattctttagactgactgaaatgaaaaggaactgttgtgtaaccatccataattccagctgtagacctgggctgtatctctatgccctgcctggcacagaccccacctcctgctccttctccctcaccaccagtcaaccttgtcctaatgaacagggagggcaaccctgaatggggagtggagggaagagatgtcatgagatggcaacgtgcacccgaagtgaggatgaaggctatgtgaatgttgtaggctgacagccgggcatagtggccccgttgccatggcgatggaggcagttgatgcgaagtgtctgcacagctcctaggatttttaacagcagctgggcagagcctcggcgtccctgaattgttgccccct

SEQ ID NO: 273 - DNA, 1974 bp, Homo Sapiens
taaaaaaaaaatagaaacatgaaagagatgtcctccttgttcaagggctaatgaccctggtgtgcgctgtctaggccccaaggtcttccttccctgctcacagcatttcaggttctccgcagctttgctgagcctgggtcaggttcggtatctgcccacatgctcacttgccacagctgtggccccatttccaaacttcagagacttaaaggtgcagctaatgatgtgcccggcctgggtcacattccctgagccctgcagacaagggagcaggaggctgagctcttatcttccacaccctgtgcacagcctgggagagttaaagcaccctagtcctatgctgcgagggccacatgccctgagaccttggaaaaaatcctacctgaattgaagagatcactatttcatcaggaggcgctgccatttcatttttcacttcggttttatcttgagtgtaaaacagcttcgcaaatcctttttcttgtttctgtaatgagcatatggtggcctcattcgtgtgataaatctgagccaccacgatatttgacttttccaatttaatttatctgaaccctctattctctggctaaaaaatatcccttacttggacttctttattttattttcaattccttaccagcactagcaggggactctgtactcatctgctggcgctgccataacaaagcactgcagcctggggggctcaaacacagaatttattctctcacagtcctagaggctagaagtccaagatcaaagtgtgggcagggtcggtttctcctgcagctctctccttggcttatagagtgccaccttctacctgtgtcttcacatcatcacctcactgagcatgtctgtgtccaaattccccttcttataagaccccagtcatactggatgaggatccacccatatgagttcattttaccttaattatctctttaacaccctgtctccaaatacagtcccattctgaggaactgagrgtaaagattcaacatatgaattttggaagggaccaattcagcccacaacaccctcttttgggatgtttattttcccccttaaggagctagttaggatgtcttatctcatgaactgactgtgaacaggaaaacagggagagaatgaagctggccaaggaacagggctggtgtcagctagcagtgcttttctgagtgagtgggtcccacagggagcttgttaaaatgcagattctgattcattaggttccagagggacctgagatttcccattctgacaagtttccagtgtgggggctgatgctgctggtccacggaccatactttgagtagcaaggagcttgatacataatgctgagtgactttcagactcctgctgtagaaaaattatgagttggctgggcgtggtggctcacgcctgtaatcccagcatttgggaggccgaggtgggcagatcacctgaggtcaggagttcgagaccagcctggccaacatggtgaaacaccatctcaccaaaaatacaaaaattagccaggtgtggtggcaggtgcctgtaatcccagctactcaggaggctgaggcaggagaatgcttgaacccgggaggcagaggttgcagtgatctgagatcgtgccactgcactccagctgggcaatagagcttgactcatctcaaaaaaaaaaaaagaaaagaaaaagaaaaattatgagttatattatcagcatatggggtgcctttcaaattgataaatttctaatattaaacctgtggatgccaaatgctgctctctgattatggcaggaaacggcacttggcagtacgaagttgctgttgggctgagctggctcatcttgttgtgcggtcctgattgcctaaagatgccttcccaggatctttactaacaatctcctgagtcatttggactttcccaacctgttatcacctctcagatgggccagccatggaggcagtcagaggagggctcgca

SEQ ID NO: 274 - DNA, 1975 bp, Homo Sapiens
tgggaacctaagataatgcgaggaccgtcatacgtgcccccaaatattggcaaaccaatgaataaatgaatgaatgagttatgaatcgctaactggctgtatttaatgaagtatgtgtgttgagccatttcccacagtgtggacagatttgtcccacatatgggcctcttcccaaaggccctaccacctaatgccatcacactggggatttgatttcaacatgtgaatttggggagatgcaaacactcagaccatagcaccatctcagtaaatgtcccactggtcactcagttcatagtgacagtgatccagccacgtcatgacaggtgccacttggcagaaacagcacagcttggaagatggcggggtgtagtcaagattccaggatccccaacgagaagccagctcttataggggagccattcatcaggattgaactctcaatcgagctggacagtaataggtgggtctgtgtattccccagatgagtatcatgacagtcacaatcctaggaaggatgtgaagcctcccccagctctcctccagttgcctgttgggcagcagagatgatggaatgtggagtctggcgtggtctgaggcctgaatccatgtgcctcatgtatgatgctcagcaagaggatctctcaattcaagggagagggcctgaatgagccttgctttccaggcctgtctgatggtccaggctgaagccctcctggcttgcactgccagacctcatccagcaggagctccttggcattgactgcttcaggatagttgcttctgctctagtgctctctaaagagcagtgctctaccatccaagctgggcttttcttttcttcttgctgatagggaaggcatgggacatgcaggatggaagtggcccccaggccttctcatgcctgggcttggtttggaaggtggtcaggtgatcaataatcctgatggcctggcattgaggagttttcctgggatgtggtcctttcrgttttttaaaaattatttttattgatacacatattgtaggtatttgtggggtgcatgtgatactttattatgtgtgtggattgtgtaatgatgaagtcagggcatttagggtctcatcaccttgattatcatttctatgtgttgagaacatttcaagttctcagttccagctattttgaaatagacagtccatttgttagctacagtcacccaacccggctgtcagacattggaacttactcctattgaactgtgtatttgtacccattcacaaactctctttgggctttcagttttacaactgggatgatcctgggaaaactaaagtaaatcagacacccgacgtgtgactaggttataatatgcccagtggaccctggggacatcttagctttcagaggtcatgctgtccaagctgactgtggggctccagaaggtggggagaggaaatgatgcaatggcccatcagaggcactacttggggcctggggccagagtgcatgtctaagcattaaggggaggggagagcagccttcataattatgaagaggagtctcaggtgcacagcttctgatgagggacagctttaattgaagacagcattgtgtaatgctcaaactccctgtcttcagagtgcctgctgtatcccaccatcagttctgtgactctccctaagcctcaattttgcatgtgttacattgggataataatagtgccaaactcatggggttgtgaggaataatgagtaaagcaattgaaaaggtttagcacaatataagtgctcaataaaagccattattattattttattacactagttttcattcctgcatagcaaattcttgcaaatgtagggactcaaaacaatataaatttattatctgacagtttttctgggtcagagtcttactaggctgtaatcagagggcaaccaaagctgtgatctcagctgaagctcaggattctcttccaagctcactggtgtt

SEQ ID NO: 275 - DNA, 601 bp, Homo Sapiens
  allele (301)..(301): n = Deletion, A or G
ccctaaaagcacaccctgcaaacctgccatgaattgacactctgtttctatcccttttccccttgtgtctgtgtctggaggaagaggataaaggacaagctgccccaagtcctagcgggcagctcgaggaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtggggnagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataactccacagtgacctggttcgccaaaggaaaagcaggcaacgtgagctgttt

SEQ ID NO: 276 - DNA, 511 bp, Homo Sapiens
  allele (256)..(256): n = Deletion or any nucleotide
ttgagcctgggaggtcaaggctgcagtgagccgagatggcgccactgcactccagcctgggcaacagagtgagaccctgtctcagaaaaaaaaaaaaaaaaaaaaaggagaggagagagactcaagcacgcccctcacaggactgctgaggccctgcaggtgtctgcagcatgtggccccaggccggggactctgtaagccactgctggagagccactcccatcctttctcccatttctctagacctgctgcctanacagtcacttttatgtggtttcgccaattttattccagctctgaaattctctgagctccccttacaagcagaggtgagctaagggctggagctcaaggcattcaaacccctaccagatctgacgaatgtgatggccacgtcccggaaatatgaagacctgttatgggcatgggagggctggcgagacaaggcggggagagccatcctccagttttacccgaaatacgtggaactcatcaaccaggctgcccggct

SEQ ID NO: 277 - DNA, 1047 bp, Homo Sapiens
aggccatcccgactcatgtttccttggccctgcccggggcacagcatcctgcccacatcyctgcagccctggctccttcctaggggctctgaggaggcagcacttggycatctggtcacagttgctgcagggcagttcttggccccagctgtaggtaaagtactgtatgttgtaattttttgaaagataacacgttcacacaactcagaattgaaatgccacagacattccccctgctccgcccctttcccccggatacccagtttctcccggaggcagccaatgatctcagaggctgtatacccmcccagagttattttatgcatatcaaggaaagtctacatagaggactgtttctggggtacccagatgcagcgtcaaatgccatggaatactacagtgaggacattatcctttcaagctttcaaatcagagcaagggaaaggtcgatgctagagtttctctagcacccatgaagccctctccctttttctactgagttttactttacaggcaacagcaggcttcaagcttggggtcattgtcgggcaacagtatctggcaagaattcaatgtctttttctcatagtcattgtattttggcctctttctatttatggcaactgagagagaaagcttattcctagatatatgtatttaagtaaaaaataaatgaattcatggaaacatattaagcaattatccagataacataagggatggcaaaaatggtgcagatggtggaggggagacaagtagaagttggggtgctcttgttgaatgtctggctctgaactctagaggaggccgcaggggctgggcaggaaggaggtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgt